(19) United States
(12) Patent Application Publication
(10) Pub. No.: US 2026/0185150 A1
(43) Pub. Date: Jul. 2, 2026

(54) METHODS AND COMPOSITIONS FOR PROCESSING AND AMPLIFICATION OF NUCLEIC ACIDS

(71) Applicant: Biomeme, Inc., Philadelphia, PA (US)

(72) Inventor: Stephen JUDICE, Dayton, ME (US)

(21) Appl. No.: 19/127,626

(22) PCT Filed: Nov. 10, 2023
(86) PCT No.: PCT/US2023/079306

§ 371 Date: May 6, 2025

Related U.S. Application Data
(60) Provisional application No. 63/424,666, filed on Nov. 11, 2022.
(60) Provisional application No. 63/501,226, filed on May 10, 2023.

(51) Int. Cl. C12Q 1/6853; C12N 9/22; C12N 15/113; C12Q 1/48; C12Q 1/6806; C12Q 1/6876

(52) U.S. Cl. CPC C12Q 1/6853; C12N 9/222; C12N 15/113; C12Q 1/48; C12Q 1/6806; C12Q 1/6876; C12Y 207/07; C12Y 301/21004; C12N 2310/20

(57) ABSTRACT

Provided herein are methods and compositions for processing a target nucleic acid sequence. The methods and compositions provided herein comprise a guide complex for recruiting an enzyme which can introduce a cut on the target nucleic acid sequence and a cut on the guide complex. The processed target nucleic acid sequence can be used in further applications such as nucleic acid amplification (e.g., isothermal amplification).

### CROSS-REFERENCE TO RELATED APPLICATIONS

This application claims priority to U.S. Provisional Patent Application No. 63/424,666, filed on Nov. 11, 2022, and U.S. Provisional Patent Application No. 63/501,226, filed on May 10, 2023, each of which is entirely incorporated herein by reference.

### BACKGROUND OF THE INVENTION

Nucleic acid amplification techniques such as polymerase chain reaction (PCR) and various isothermal amplification techniques have become an integral part of nucleic acid-based diagnostics and research techniques.

### SUMMARY OF THE INVENTION

Recognized herein is a need for improved methods and compositions for processing target nucleic acid molecules with high efficiency and/or simplified optimization process for reaction conditions. The methods and compositions described herein can be used for generating initial products for downstream applications such as isothermal amplifications.

In an aspect, the present disclosure provides a method of processing a single-stranded nucleic acid molecule comprising a target sequence, the method comprising: (a) contacting the single-stranded nucleic acid molecule with a guide complex comprising a guide polynucleotide under conditions where the guide polynucleotide hybridizes to the single-stranded nucleic acid molecule, wherein the guide polynucleotide comprises: (i) a non-target binding region comprising a restriction endonuclease recognition sequence for an enzyme that is a type IIs restriction enzyme, (ii) a target binding region configured to hybridize to the target sequence, and (iii) a blocked 3′ end non-extendable by a polymerase; and (b) introducing the type IIs restriction enzyme under conditions sufficient to cause the type IIs restriction enzyme to bind the restriction endonuclease recognition sequence and cut within the target sequence.

In some embodiments, in (b) the cut exposes an extendable 3′ end of the target sequence.

In some embodiments, the method further comprises extending the extendable 3′ end using a polymerase.

In another aspect, the present disclosure provides a method of amplifying a single-stranded nucleic acid molecule comprising a target sequence, the method comprising: (a) contacting the single-stranded nucleic acid molecule with a guide complex comprising a guide polynucleotide under conditions where the guide polynucleotide hybridizes to the single-stranded nucleic acid molecule, wherein the guide polynucleotide comprises: (i) a non-target binding region comprising a restriction endonuclease recognition sequence for a type IIs restriction enzyme, (ii) a target binding region configured to hybridize to the target sequence, and (iii) a blocked 3′ end non-extendable by a polymerase; (b) introducing the type IIs restriction enzyme under conditions sufficient to cause the type IIs restriction enzyme to bind the restriction endonuclease recognition sequence and cut within the target sequence to generate an extendable 3′ end; and (c) extending the extendable 3′ end of the target sequence using a polymerase.

In some embodiments, the guide polynucleotide is a first guide polynucleotide, and the guide complex comprises a second guide polynucleotide, wherein the second guide polynucleotide comprises (i) a non-target binding region that is complementary with the non-target binding region of the first guide polynucleotide and (ii) a target binding region configured to hybridize to the target sequence.

In some embodiments, when the first guide polynucleotide of the guide complex is hybridized to the target polynucleotide sequence, the target binding region of the second guide polynucleotide is not hybridized to the target sequence. In some embodiments, the first guide polynucleotide and the second guide polynucleotide hybridize to form a dimer. In some embodiments, the first guide polynucleotide and the second guide polynucleotide hybridize via the non-target binding region of the first guide polynucleotide and the second guide polynucleotide to form the dimer having a double-stranded binding region.

In some embodiments, the double-stranded binding region comprises the restriction endonuclease recognition sequence.

In some embodiments, the type IIs restriction enzyme binds to the double-stranded binding region of the dimer.

In another aspect, the present disclosure provides a method of amplifying a single-stranded nucleic acid molecule comprising a target sequence, the method comprising: (a) contacting a guide complex with the single-stranded nucleic acid molecule, wherein the guide complex comprises: (i) a first guide polynucleotide comprising, from 5′ to 3′, a non-target binding region and a target binding region that hybridizes with the target sequence of the single-stranded nucleic acid molecule, and (ii) a second guide polynucleotide that hybridizes with the non-target binding region of the first guide molecule to form a double-stranded binding region, wherein the double-stranded binding region binds to an enzyme; (b) cutting the target sequence using the enzyme to expose an extendable 3′ end of the target sequence; (c) extending the extendable 3′ end of the target sequence with a polymerase to generate an extension product, wherein the extension product displaces the second guide polynucleotide; (d) cutting the first guide polynucleotide within the target binding region to expose an extendable 3′ end of the first guide polynucleotide; and (e) extending the extendable 3′ end of the first guide polynucleotide using the polymerase to generate a complementary molecule of the target sequence of the single-stranded nucleic acid molecule, thereby amplifying the single-stranded nucleic acid molecule.

In some embodiments, the second guide polynucleotide comprises, from 5′ to 3′ (i) a non-target binding region that hybridizes with the non-target binding region of the first guide polynucleotide and (ii) a target binding region configured to hybridize with the target sequence.

In some embodiments, the method further comprises, prior to (b), cutting the first guide polynucleotide within the target binding region using the enzyme, wherein the guide complex dissociates from the single-stranded nucleic acid molecule.

In some embodiments, the method further comprises repeating (d) and (e) to generate a plurality of complementary molecules of the target sequence of the single-stranded nucleic acid molecule.

In some embodiments, an additional guide complex binds to the complementary molecule.

In some embodiments, the method further comprises using the complementary molecule with the additional guide complex bound thereto as a starting template to generate copies of the target molecule.

In some embodiments, the enzyme is a type IIs restriction enzyme. In some embodiments, the type IIs restriction enzyme comprises N.BstNBI, N.Bst9 I, N.BspD6I, a functional fragment thereof, or a combination thereof.

In some embodiments, the guide polynucleotide comprises a blocked 3′ end non-extendable by a polymerase. In some embodiments, the blocked 3′ end comprises a PNA, a modified base, a phosphate group, a ddNTP, a solid support, a spacer, or any combination thereof.

In some embodiments, the single-stranded nucleic acid molecule with the cut and the guide polynucleotide bound thereto is used as a starting template for an amplification. In some embodiments, the amplification is an isothermal amplification. In some embodiments, the enzyme exhibits a high-frequency endonuclease activity. In some embodiments, the high-frequency endonuclease activity is from a large subunit of the enzyme. In some embodiments, the enzyme exhibits a low-frequency endonuclease activity. In some embodiments, the low-frequency endonuclease activity is from a small subunit of the enzyme. In some embodiments, the enzyme exhibits at least two differential enzymatic activity rates. In some embodiments, the at least two differential enzymatic activity rates comprise two differential endonuclease activity rates when cutting two different cutting sites.

In some embodiments, one of the two differential endonuclease activity rates comprises cutting the target sequence of the single-stranded nucleic acid molecule with low frequency. In some embodiments, one of the two differential endonuclease activity rates comprises cutting the target binding region of the guide polynucleotide with high frequency. In some embodiments, the two differential endonuclease activity rates are asymmetric or non-equal.

In some embodiments, the enzyme comprises BsmAI, Nt.BsmAI, Transcription Activator-Like Effector Nucleases, N.Bst9 I, N.BspD6I, Nt.BspQI, Nb.BbvCI, Nb.BsmI, Nb.BssSI, Nb.BsrDI, Nb.BtsI, Nt.AlwI, Nt.BbvCI, Nt.BstNBI, Nt.CviPII, Nb. Mva1269I, Nb.Bpu10I, and Nt.Bpu10I, a functional fragment thereof, or a combination thereof.

In some embodiments, a temperature is changed over a course of the method.

In some embodiments, a first activity rate of the at least two differential enzymatic activity rates is favored at a first temperature, and a second activity rate of the at least two differential enzymatic activity rates is favored at a second temperature different from the first temperature.

In some embodiments, the enzyme comprises two different active sites or endonuclease domains conferring at least two differential enzymatic activities.

In some embodiments, the target sequence comprises a recognition site specifically recognized by the enzyme or a first activity of the at least two differential enzymatic activities of the enzyme to introduce a cut.

In some embodiments, the target binding region of the guide polynucleotide comprises a recognition site specifically recognized by the enzyme or a second activity of the at least two differential enzymatic activities of the enzyme to introduce a cut.

In some embodiments, the target binding region is at least about 12 to about 25 nucleotides in length.

In some embodiments, a concentration of the guide polynucleotide is at least about 0.1 μM, at least about 1 μM, or about 0.1 μM to about 4 μM.

In some embodiments, the non-target binding region comprises a palindromic sequence. In some embodiments, the non-target binding region is self-complementary. In some embodiments, the non-target binding region is at least about 12 nucleotides in length.

In some embodiments, the single-stranded nucleic acid molecule is a single-stranded deoxyribonucleic acid (ssDNA) or a single-stranded ribonucleic acid (ssRNA).

In some embodiments, the target binding region comprises at least one peptide nucleic acid (PNA) residue. In some embodiments, the polymerase has strand displacement activity. In some embodiments, the guide polynucleotide or the first guide polynucleotide further comprises an additional non-target binding region. In some embodiments, the additional non-target binding region is located at a 5′ end of the guide polynucleotide or the first guide polynucleotide.

In some embodiments, the additional non-target binding region comprises an additional restriction endonuclease recognition sequence for an additional enzyme. In some embodiments, the additional enzyme is the same or different from the enzyme. In some embodiments, the additional non-target binding region blocks the 3′ end of the guide polynucleotide or the first guide polynucleotide from extension. In some embodiments, the single-stranded nucleic acid molecule comprises two or more single-stranded nucleic acid molecules, each single-stranded nucleic acid molecule comprising a different target sequence. In some embodiments, the two or more single-stranded nucleic acid molecules are contained within a single reaction mixture.

In some embodiments, the method of amplifying a single-stranded nucleic acid molecule shortens a cycle threshold value or a time to result value in a nucleic acid amplification compared to a cycle threshold value or a time to result value in a nucleic acid amplification of an otherwise identical method of amplifying the single-stranded nucleic acid molecule without the guide complex.

In some embodiments, the method of amplifying a single-stranded nucleic acid molecule shortens a cycle threshold value or a time to result value in a nucleic acid amplification compared to a cycle threshold value or a time to result value in an existing nucleic acid amplification method.

In some embodiments, the existing nucleic acid amplification method is selected from the group consisting of loop-mediated isothermal amplification (LAMP), helicase-dependent amplification (HDA), rolling circle amplification (RCA), multiple displacement amplification (MDA), recombinase polymerase amplification (RPA), and nucleic acid sequence-based amplification (NASBA). In some embodiments, the cycle threshold value is at most 30.

In another aspect, the present disclosure provides a polynucleotide-polypeptide complex comprising: a single-stranded nucleic acid molecule having bound thereto a guide complex, wherein the guide complex comprises: (i) a first guide polynucleotide comprising, from 5′ to 3′, a non-target binding region and a target binding region that hybridizes with a target sequence of the single-stranded nucleic acid molecule, and (ii) a second guide polynucleotide that hybridizes with the non-target binding region of the first guide molecule to form a double-stranded binding region, wherein the double-stranded binding region comprises a restriction endonuclease recognition sequence for an enzyme that is a type IIs restriction enzyme.

In another aspect, the present disclosure provides a system of processing a single-stranded nucleic acid molecule comprising a target sequence, the system comprising: the single-stranded nucleic acid molecule having bound thereto a guide complex comprising a guide polynucleotide, wherein the guide polynucleotide comprises: (i) a non-target binding region comprising a restriction endonuclease recognition sequence for an enzyme that is a type IIs restriction enzyme, (ii) a target binding region configured to hybridize to the target sequence, and (iii) a blocked 3′ end non-extendable by a polymerase; and the enzyme bound to the restriction endonuclease recognition sequence of the non-target binding region.

In another aspect, the present disclosure provides a kit comprising a guide complex or a guide polynucleotide described herein.

In some embodiments, the kit further comprises a probe or a dye for detecting an amplification product generated using the kit.

In some embodiments, the kit further comprises an informational material describing an instruction of using the kit.

In another aspect, the present disclosure provides a system for processing a plurality of single-stranded nucleic acid molecules, each comprising a different target sequence, the system comprising: a first single-stranded nucleic acid molecule wherein the first single-stranded nucleic acid molecule is bound to a first guide complex comprising a first guide polynucleotide, wherein the first guide polynucleotide comprises: (i) a first non-target binding region comprising a first restriction endonuclease recognition sequence for an enzyme that is a type IIs restriction enzyme; (ii) a first target binding region configured to hybridize to a first target sequence; and (iii) a first blocked 3′ end non-extendable by a polymerase; and a second single-stranded nucleic acid molecule wherein the second single-stranded nucleic acid molecule is bound to a second guide complex comprising a second guide polynucleotide, wherein the second guide polynucleotide comprises: (i) a second non-target binding region comprising a second restriction endonuclease recognition sequence for the enzyme that is a type IIs restriction enzyme; (ii) a second target binding region configured to hybridize to a second target sequence; and (iii) a second blocked 3′ end non-extendable by a polymerase; wherein the enzyme that is a type IIs restriction enzyme binds to the first restriction endonuclease recognition sequence of the first non-target binding region or the second restriction endonuclease recognition sequence of the second non-target binding region.

In some embodiments, the system further comprises a third single-stranded nucleic acid molecule wherein the third single-stranded nucleic acid molecule is bound to a third guide complex comprising a third guide polynucleotide, wherein the third guide polynucleotide comprises: (i) a third non-target binding region comprising a third restriction endonuclease recognition sequence for an enzyme that is a type IIs restriction enzyme; (ii) a third target binding region configured to hybridize to a third target sequence; and (iii) a third blocked 3′ end non-extendable by a polymerase; wherein the enzyme that is a type IIs restriction enzyme binds to the third restriction endonuclease recognition sequence of the third non-target binding region.

In some embodiments, the system further comprises a fourth single-stranded nucleic acid molecule wherein the fourth single-stranded nucleic acid molecule is bound to a fourth guide complex comprising a fourth guide polynucleotide, wherein the fourth guide polynucleotide comprises: (i) a fourth non-target binding region comprising a fourth restriction endonuclease recognition sequence for an enzyme that is a type IIs restriction enzyme; (ii) a fourth target binding region configured to hybridize to a fourth target sequence; and (iii) a fourth blocked 3′ end non-extendable by a polymerase; wherein the enzyme that is a type IIs restriction enzyme binds to the fourth restriction endonuclease recognition sequence of the fourth non-target binding region.

In some embodiments, the first single-stranded nucleic acid molecule and the second single-stranded nucleic acid molecule are from different samples. In some embodiments, the different samples comprise samples obtained from a bacterium, a virus, a human, or any combination thereof. In some embodiments, the bacterium is selected from the group consisting of Neisseria gonorrhoeae, Chlamydia trachomatis, and Trichomonas vaginalis. In some embodiments, the virus is selected from the group consisting of a double-stranded DNA virus, a single-stranded DNA virus, a double-stranded RNA virus, a single-stranded RNA virus, a positive sense single-stranded reverse transcriptase virus, and a double-stranded DNA reverse transcriptase virus.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1O show an example of precursor steps leading to an isothermal amplification cycle according to various embodiments described herein. FIG. 1A depicts the duplexed oligo complex binding to the target nucleic acid strand. FIG. 1B depicts endonucleolytic activity on the duplexed oligo/target complex. FIG. 1C depicts a polymerase extending off of the 3′ end of the target strand. FIG. 1D depicts the polymerase displacing the duplexed guide molecule. FIG. 1E depicts endonucleolytic activity on the oligo/extension product complex. FIG. 1F depicts a polymerase extending off the 3′ end of the cut oligo and displacement of the guide. FIG. 1G depicts endonuclease activity on the newly synthesized portion complementary to the target strand. FIG. 1H depicts a polymerase extending off the 3′ end of the cut site and displacement of the synthesized complement to the target strand. FIG. 1I depicts the displaced complement acting as a new target for the second complementary strand duplexed oligo complex. FIG. 1J depicts the polymerase displacing the second complementary strand duplexed guide molecule.

FIG. 1K depicts the completed extension on the new guide molecule. FIG. 1L depicts endonucleolytic activity on the second complementary strand oligo/extension product complex.

FIG. 1M depicts a polymerase extending off the 3′ end of the cut site of the second complementary strand of the oligo/extension product complex. FIG. 1N depicts endonucleolytic activity on the newly synthesized complementary strand of the second complementary strand guide. FIG. 1O depicts the displaced and single stranded synthesized fragments as starting material for a strand displacement amplification reaction.

FIGS. 2A-2C show a system for the creation of products suitable for amplification by isothermal amplification reactions using a guide molecule with a point mutation relative to the target sequence. FIG. 2A depicts a guide molecule with a point mutation binding to a target DNA and an endonuclease cutting the target. FIG. 2B depicts extension off the target at the 3′ end and an endonuclease cutting the guide molecule. FIG. 2C depicts the displacement of the guide complementary to the target after endonucleolytic cutting with subsequent synthesis of a new strand. The opposite strand guide extension will only see the base that originated from the target and not the base that exists in the guide oligo sequence due to the lack of extension off the end of the guide oligo.

FIGS. 3A-3B show a control experiment where there is no mismatch between the guide and primer. FIG. 3A depicts the guide oligos, probes, and target sequence used in the control experiment with no mismatch between guide oligos and target. FIG. 3B depicts the amplification result of the control reaction without a point mutation.

FIGS. 4A-4B show an experiment where there is an A to C mismatch between the guide and primer. FIG. 4A depicts the guide oligos, probes, and target sequence used in the mismatch experiment. FIG. 4B depicts the amplification resulting in probe signal that is of target origin; rather than probe signal that is of guide oligo origin indicative of an asymmetric endonuclease activity.

FIGS. 5A-5B show a control experiment where there is no mismatch between the guide and primer. FIG. 5A depicts the guide oligos, probes, and target sequence used in the control experiment. FIG. 5B depicts the amplification result of the control reaction without a point mutation.

FIGS. 6A-6B show an experiment where there is an A to C mismatch between the guide and primer. FIG. 6A depicts the guide oligos, probes, and target sequence used in the mismatch experiment. FIG. 6B depicts the amplification resulting in probe signal that is of target origin; rather than probe signal that is of guide oligo origin indicative of an asymmetric endonuclease activity.

FIGS. 7A-7D illustrate an experiment which uses internal fluorescence to detect the formation of double-stranded nucleic acids and the use of different guides. FIG. 7A depicts a single-stranded DNA (ssDNA) molecule with a 5′ quencher and an internal fluorescein-T. FIG. 7B depicts quenched fluorescence when the strand is self-complemented. FIG. 7C depicts the binding of a guide molecule to the target ssDNA. FIG. 7D depicts the cut sites which will initiate the formation of extension products and the formation of fluorescent double-stranded nucleic acids.

FIGS. 8A-8D show the amplification/primer extension results of different primers using Bst polymerase. The 3′ extension of the guide molecule is blocked when encountering a 2′O methyl RNA base or a phosphorylated base. FIG. 8A depicts the amplification/primer extension reaction results using only Bst polymerase. FIG. 8B depicts the amplification/primer extension reaction results using Bst polymerase and endonuclease Nt.BsmAI. FIG. 8C depicts the amplification/primer extension reaction results using Bst polymerase and the endonucleases Nt.BsmAI and N.BstNBI. FIG. 8D depicts the amplification/primer extension reaction results using Bst polymerase and endonuclease N.BstNBI.

FIGS. 9A-9D show the amplification/primer extension reaction results of different primers using Bst polymerase. The 3′ extension of the guide molecule is blocked when encountering a 2′O methyl RNA base or a phosphorylated base. FIG. 9A depicts the amplification/primer extension reaction results using only Bst polymerase. FIG. 9B depicts the amplification/primer extension reaction results using Bst polymerase and endonuclease Nt.BsmAI. FIG. 9C depicts the amplification/primer extension reaction results using Bst polymerase and the endonucleases Nt.BsmAI and N.BstNBI. FIG. 9D depicts the amplification/primer extension reaction results using Bst polymerase and endonuclease N.BstNBI.

FIGS. 10A-10B illustrate the cycle threshold results of loop-mediated isothermal amplification (LAMP). FIG. 10A depicts cycle threshold results of LAMP when comparing LAMP to LAMP with differential targeted endonuclease cutting technology (DTECT) priming and DTECT priming on its own. FIG. 10B is a zoomed-in version of FIG. 10A which more clearly shows the difference between the LAMP with DTECT priming and DTECT priming cycle threshold results.

FIGS. 11A-11D illustrate an experiment which uses internal fluorescence to detect the formation of double-stranded nucleic acids and the use of different guides. FIG. 11A depicts a single-stranded DNA (ssDNA) molecule with a 5′ quencher and an internal fluorescein-T. FIG. 11B depicts quenched fluorescence when the strand is self-complemented. FIG. 11C depicts a 2′O methyl bases on the guide molecule. FIG. 11D depicts the cut sites which will initiate the formation of extension products and the formation of fluorescent double-stranded nucleic acids.

FIGS. 12A-12F show the amplification results of primer guide C (FIG. 12A), primer guide D (FIG. 12B), primer guide E (FIG. 12C), primer guide H (FIG. 12D), primer guide F (FIG. 12E), and primer guide G (FIG. 12F) under different conditions using Bst polymerase, Bst, and Nt.Bsp.

FIGS. 13A-13B show a comparison between the use of primer guide F, which is unblocked and has a methoxylation block on the guide (FIG. 13A), and primer guide C, which is unblocked and extendable (FIG. 13B).

FIGS. 14A-14E show the results of isothermal SDA after the production of a restriction digestion monkeypox target product. FIG. 14A depicts a 10-fold dilution series using primer guides with blocks. FIG. 14B depicts a 1×10⁶ copy number per reaction 10-fold dilution series using primer guides with blocks. FIG. 14C depicts a summary of the log copy number per reaction of monkeypox primer guide with block amplification reactions. FIG. 14D depicts monkeypox virus amplification in a NP matrix direct amplification procedure using primer guides with blocks. FIG. 14E depicts a summary of the data from FIG. 14D.

FIGS. 15A-15B show the results of a triplex isothermal amplification reaction. FIG. 15A shows reaction conditions from a triplex isothermal reaction, wherein Isofast BST is a DNA polymerase, N.BstNBI is a site specific endonuclease that primarily cleaves only one strand of DNA on a double-stranded DNA substrate; AMV rt enz refers to Avian Myeloblastosis Virus reverse transcriptase; dNTP refers to deoxynucleoside triphosphate; NaSO₄ is sodium sulphate; MgSO₄ is magnesium sulfate; Tris is tris(hydroxymethyl)aminomethane, (NH₄)₂SO₄ is ammonium sulfate; NG refers to Neisseria gonorrhoeae; CT refers to Chlamydia trachomatis, RPP stands for ribosomal protection protein; rt stands for reverse transcriptase; Cy5 refers to Cyanine-5; HEX refers to hexachlorofluorescein, and Fam refers to fluorescein amidite. FIG. 15B shows preliminary performance results of XCEL triplex isothermal reaction using N. gonorrhoeae, C. trachomatis, and Human RPP30. Approximately 75 IFU per reaction for C. trachomatis and approximately 150 CFU per reaction for N. gonorrhoeae were used.

FIG. 16A depicts the results from a tetraplex isothermal amplification reaction using Trichomonas vaginalis, N. gonorrhoeae, C. trachomatis, and Ribonuclease P/MRP subunit p30 (RPP30). M1 purified T. vaginalis, N. gonorrhoeae, and C. trachomatis had an RNA titration of 250 picograms per reaction of purified human RNA. FIG. 16B shows reaction conditions from a tetraplex isothermal reaction, wherein Lucigen BST is a DNA polymerase, N.BstNBI is a site specific endonuclease that primarily cleaves only one strand of DNA on a double-stranded DNA substrate; AMV rt enz refers to Avian Myeloblastosis Virus reverse transcriptase; dNTP refers to deoxynucleoside triphosphate; NaSO₄ is sodium sulphate; MgSO₄ is magnesium sulfate; Tris is tris(hydroxymethyl)aminomethane, (NH₄)₂SO₄ is ammonium sulfate; NG refers to N. gonorrhoeae; CT refers to C. trachomatis, Tv refers to T. vaginalis; RPP stands for ribosomal protection protein; rt stands for reverse transcriptase; Cy5 refers to Cyanine-5; HEX refers to hexachlorofluorescein, and Fam refers to fluorescein amidite.

FIG. 17 shows a computer system that is programmed or otherwise configured to implement methods provided herein.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. It is appreciated that although the vial caps are described in the Figures as having a configuration comprising three void filling caps filling three vials in linear arrangement, that such description is merely illustrative as the inventive concepts described herein contemplate various configurations and numbers of void filling caps.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, or within 2-fold, of a value. Where particular values are described in the application and claims, unless otherwise stated the term “about” meaning within an acceptable error range for the particular value may be assumed.

### Overview

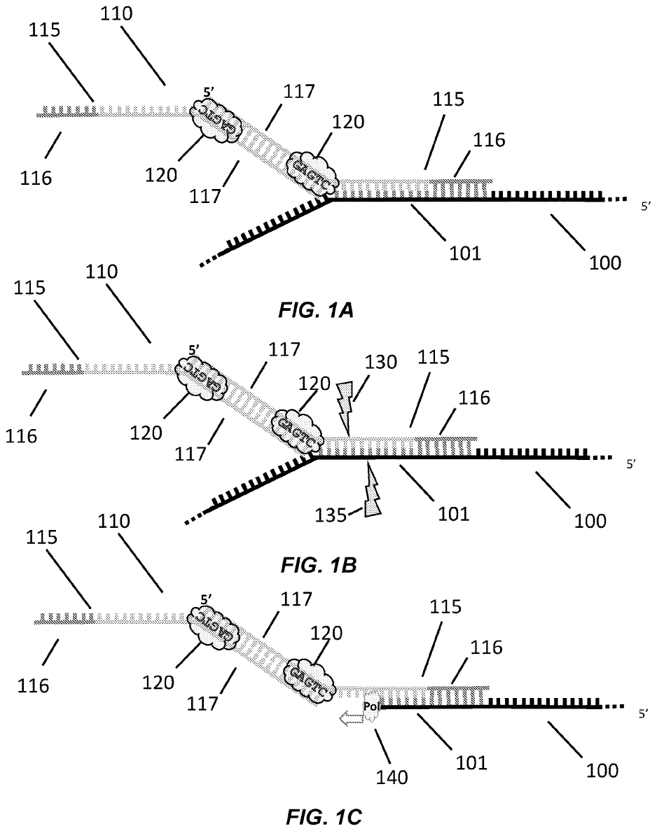

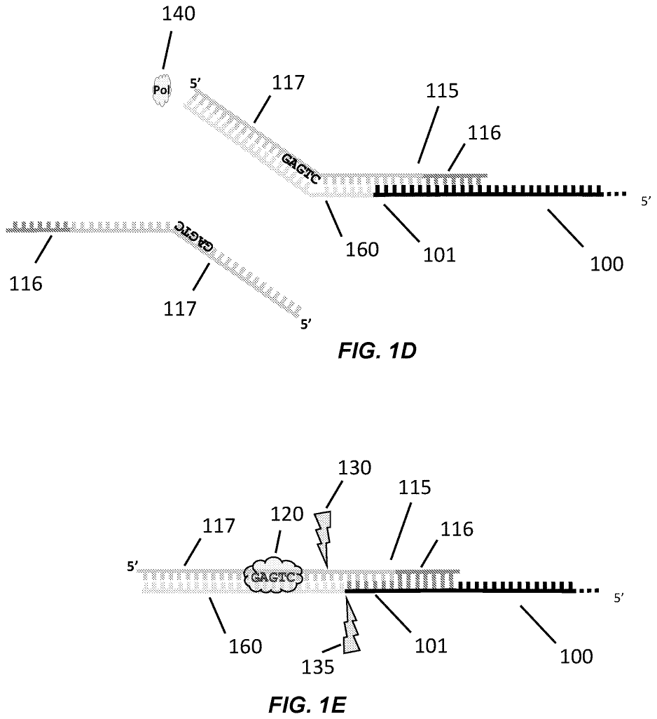

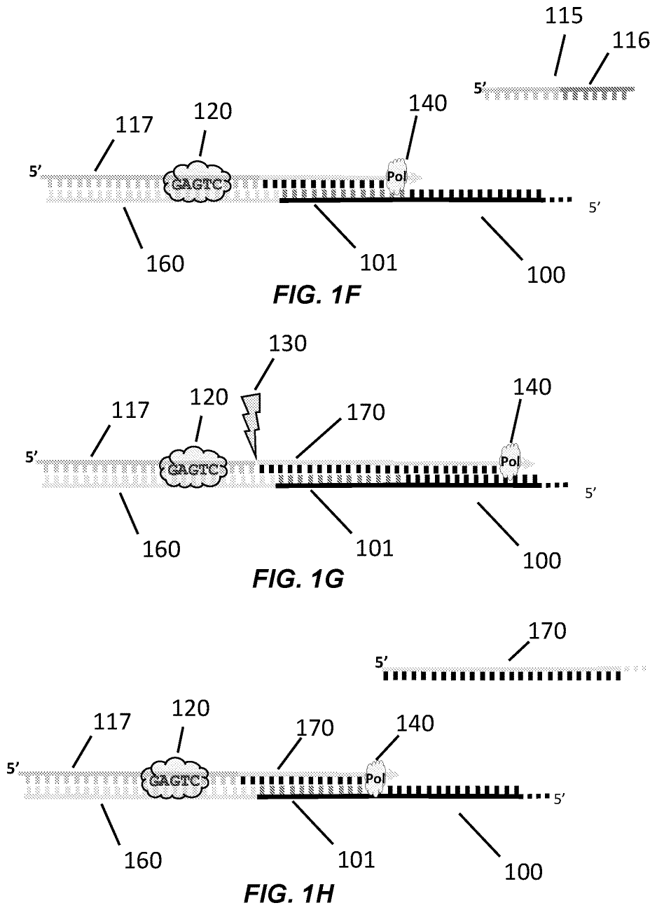

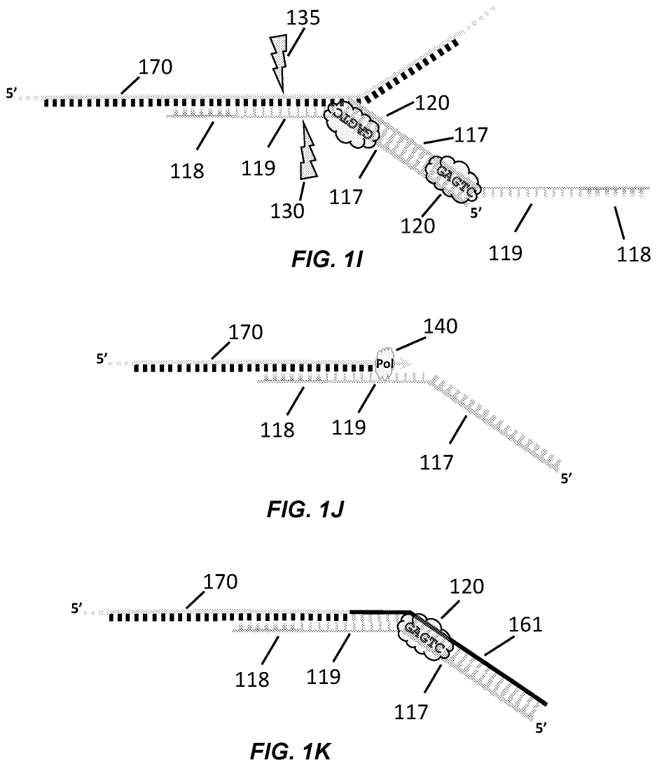

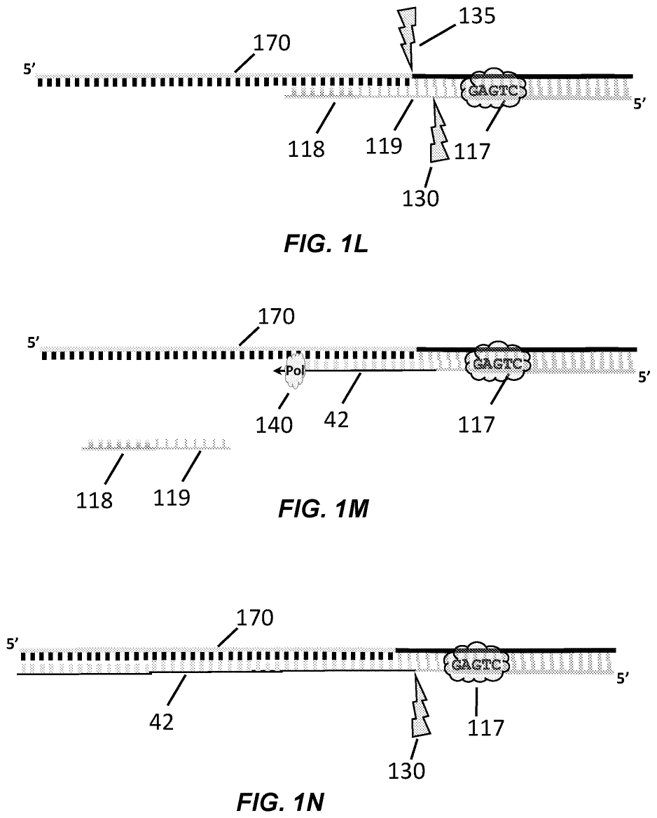

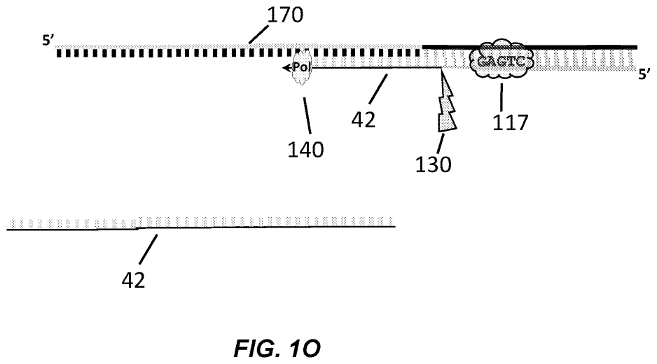

The present disclosure provides methods, systems, compositions, and kits for processing target nucleic acid molecules. In some aspects, the present disclosure provides for methods of amplification of nucleic acids (e.g., isothermal amplification). Such a method can involve a cycle of steps such as that depicted in FIGS. 1A through 1O. The methods provided herein can offer higher amplification efficiency and easier optimization procedure compared with existing amplifications (e.g. isothermal amplifications). The processed target nucleic acid molecules can be used in various amplification reactions not limited to the amplification or processing methods described herein.

Such a method can start with the formation of a structure such as that depicted in FIG. 1A, in which a guide nucleic acid complex (or a guide complex) is formed to direct a restriction enzyme to a predetermined site in a nucleic acid. FIG. 1A depicts a nucleic acid strand (e.g., a single-stranded DNA strand or ssDNA strand) (100) comprising a target nucleic acid sequence (101). In some cases, the ssDNA strand can be generated by reverse transcribing a target RNA sequence. In some cases, the ssDNA strand can be generated by denaturing a double-stranded DNA (dsDNA) sequence. In FIG. 1A, a type IIs restriction enzyme (120) is directed to the vicinity of the target site via formation of a guide complex. This guide nucleic acid complex is constituted via self-annealing of single copies of a guide polynucleotide which comprise: a non-target binding region comprising a restriction endonuclease recognition sequence for a type IIs restriction enzyme (117), a target binding region configured to hybridize to the target sequence (115), and a blocked 3′ end non-extendable by a polymerase (116). Note that in FIG. 1A, self-annealing of the two copies of the guide polynucleotide forms a double-stranded palindromic region that permits binding of the type II restriction enzyme in the vicinity of the target site.

Such a method can continue in a second stage with the process depicted in FIG. 1B and FIG. 1C. After the type IIs restriction enzyme (120) is directed to the vicinity of the target site (101) by the double-stranded palindromic region (two copies of 117) formed by self-annealing of the guide polynucleotides, the type IIs restriction enzyme is able to, characteristic to its activity, cleave single-stranded locations (130, 135) distal to its binding site (FIG. 1B). One of these cleavable single-stranded locations (135) is on the nucleic acid strand (101) that comprises the target nucleic acid sequence (101). The other cleavable single-stranded location (130) is located on the guide polynucleotide itself (130). If selective enzymatic conditions, an engineered polymerase, or BspD6I is used, cleavage at one of the sites (e.g. the single-stranded site on the nucleic acid strand (101) that comprises the target nucleic acid sequence (101)) can be favored. Cleavage at the single-stranded site on the nucleic acid strand (101) that comprises the target nucleic acid sequence (101) generates a free 3′ hydroxyl that can then be extended by a strand-displacing polymerase present in the reaction.

Such a method can continue in a third stage with the process depicted in FIG. 1D through FIG. 1F. Extension of the free 3′ hydroxyl by the strand-displacing polymerase (140, FIG. 1C) produces a region (160) of the nucleic acid strand (101) that comprises the target nucleic acid sequence (101) that is complementary to the restriction endonuclease recognition sequence for the type IIs restriction enzyme (117) from the guide polynucleotide (FIG. 1D). Extension of the nucleic acid (100) displaces the second copy of the guide polynucleotide (116/117, lower molecule), that previously formed half of the guide complex. Extension of the nucleic acid (100) with the region complementary to the restriction endonuclease recognition sequence for the type IIs restriction enzyme (160) forms a new double-stranded structure where a type IIs restriction enzyme (120) can bind (FIG. 1E). As in the second stage, the type IIs restriction enzyme is able to cleave single-stranded locations (130, 135) distal to its binding site (FIG. 1E). While cleavage at the single-stranded site (135) that contains the target nucleic acid site (100) causes the strand (100) to merely be extended again by the polymerase, cleavage at the single-stranded site (130) allows for a new procedure to commence (FIG. 1E). Specifically, cleavage at site 130 of FIG. 1E on the annealed guide polynucleotide removes the sequence containing the blocked 3′ end (116) and allows the guide polynucleotide to be extended to comprise a sequence (170) complementary to the strand (100) containing the target nucleic acid site (101) (FIG. 1F).

Such a method can continue in a fourth stage with the process depicted in FIG. 1G and FIG. 1H. As the double-stranded structure of FIG. 1G no longer comprises a blocked 3′ end, repeated cleavage at site 130 of FIG. 1G liberates a single strand comprising a sequence (170) complementary to the strand (100) containing the target nucleic acid site (101), and then allows extension of a new strand (171) to replace it. Additionally, the liberated strand (170) can further serve as a new template analogously to the strand 100 of FIG. 1A (FIG. 1I), which allows for strand 170 to be further cleaved and repeatedly extended as in FIG. 1H (FIG. 1J). FIG. 1K depicts an exemplary completed extension on the new guide molecule.

In some cases, the method can continue, as seen in FIG. 1L, wherein endonucleolytic activity can occur on the second complementary strand oligo/extension product complex (170). FIG. 1M depicts a polymerase (140) extending of the 3′ end of the cut site of the second complementary strand of the oligo/extension product complex. Endolytic activity on the newly synthesized strand (130) occurs (FIG. 1N) and the displaced, single-stranded synthesized fragment (42) of FIG. 1O can serve as starting material for additional strand displacement amplification reactions.

In some cases, methods according to the disclosure do not involve amplification and utilize the structure depicted in FIG. 1A to direct cleavage of a single-stranded nucleic acid molecule (100) containing a target site (101) at a specified position (135, FIG. 1B).

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, generally refers to a base-sugar-phosphate combination. A nucleotide may comprise a synthetic nucleotide. A nucleotide may comprise a nucleotide analog. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. Synthetic nucleotide analogs may include locked nucleic acids (LNAs), bridged nucleic acids (BNAs), fluorinated nucleic acids (also known as fluoro-modified nucleic acids), and peptide nucleic acids (PNAs). As used herein, the term “locked nucleic acid” (“LNA”), generally refers to a nucleic acid analog wherein the ribose ring is “locked” with an extra bridge connecting the 2′-oxygen atom with the 4′-carbon atom of the nucleotide such as a methylene bridge (see e.g. WO 99/14226, which is incorporated by reference in its entirety herein). As used herein, the term “bridged nucleic acid (BNA),” generally refers to constrained or inaccessible nucleic acid molecules which have a fixed bridge structure at the 2′- or 4′-position. As used herein, “fluorinated nucleic acids” generally refer to nucleic acids which have incorporated a fluorine atom, often at the 2′- or 4′-position. As used herein, the term “peptide nucleic acid (PNA),” generally refers to a nucleotide analog wherein the backbone of the analog, for example a sugar backbone in DNA, is a pseudopeptide. A PNA backbone can comprise, for example, a sequence of repeated N-(2-amino-ethyl)-glycine units. A peptide nucleic acid analog can react as DNA would react in a given environment, and can additionally bind complementary nucleic acid sequences and various proteins. Due to the non-natural backbone, PNAs can be insensitive to endonuclease cleavage in situations where an endonuclease would cleave the equivalent DNA/RNA sequence and in addition, confer specificity and binding to complementary DNA under varying salt conditions. The term “nucleotide,” as used herein, may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Detectable labels may include, for example, radioactive isotopes, fluorescent labels, chemiluminescent labels, bioluminescent labels and enzyme labels.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be DNA. A polynucleotide may be RNA. A polynucleotide may comprise one or more nucleotide analogs (e.g., including those with an altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, wyosine, PNAs, and LNAs.

As used herein, the term “restriction endonuclease,” “restriction enzyme,” or grammatical equivalents thereof generally refers to an enzyme that originates in bacterial host defense and is understood to recognize a specific sequence on an incoming viral DNA and cleave the DNA either at the recognition sequence or at a distinct sequence site. One group of restriction endonucleases are identified as Type IIS. This group can recognize asymmetric DNA sequences and cleaves the DNA at a site outside the cleavage site that is at a defined distance from the recognition site. In some cases, type IIS restriction endonucleases cleave DNA between 1 and 20 nucleotides from the relevant recognition site.

As used herein, the term “restriction endonuclease recognition sequence” generally refers to a location on a nucleic acid molecule (e.g., DNA molecule) containing specific sequences of nucleotides, which are recognized by various restriction enzymes. These sequences can comprise from 4-8 base pairs to 12-40 base pairs in length. These sites can be palindromic sequences.

As used herein, the term “polymerase” generally refers to an enzyme that produces a complementary replicate of a nucleic acid molecule using the nucleic acid as a template strand. DNA polymerases bind to the template strand and then move down the template strand adding nucleotides to the free hydroxyl group at the 3′ end of a growing chain of nucleic acid. DNA polymerases synthesize complementary DNA molecules from DNA (e.g., DNA-dependent DNA polymerases) or RNA templates (e.g., RNA-dependent DNA polymerases or reverse transcriptases) and RNA polymerases synthesize RNA molecules from DNA templates (e.g., DNA-dependent RNA polymerases which participate in transcription). DNA polymerases generally use a short, preexisting RNA or DNA strand, called a primer, to begin chain growth; and some DNA polymerases can utilize any free 3′ hydroxyl in a DNA duplex for extension. Some DNA polymerases replicate single-stranded templates, while other DNA polymerases displace the strand upstream of the site where they add bases to a chain.

As used herein, the term “strand displacing,” when used in reference to a polymerase, generally refers to an activity that removes a complementary strand from base-pairing with a template strand being read by the polymerase. Example polymerases having strand displacing activity include the large fragment of Bacillus stearothermophilus polymerase (Bst polymerase), exo-Klenow polymerase, Bst 2.0 polymerase, Bst 3.0 polymerase, SD DNA polymerase, phi29 DNA polymerase, sequencing-grade T7 exo-polymerase, and OmniTaq 2 LA DNA polymerase.

As used herein, the terms “amplify,” “amplifies,” “amplified,” “amplification,” and “amplicon” generally refer to any method for replicating a nucleic acid. The replication can be conducted with the use of a primer-dependent polymerase. The replication can be enzyme-free amplification. In some cases, amplifying or replicating a target nuclei acid strand also comprises replicating or amplifying a complementary strand of the target nucleic acid strand. Amplified products can be subjected to subsequence analyses, including but not limited to melting curve analysis, nucleotide sequencing, single-strand conformation polymorphism assay, allele-specific oligonucleotide hybridization, Southern blot analysis, and restriction endonuclease digestion.

The terms “hybridizes,” and “annealing,” as used herein, generally refer to a reaction in which one or more polynucleotides interact to form a complex that is stabilized via hydrogen bonding between the bases of the nucleotide residues. The hydrogen bonding may occur by Watson Crick base pairing, Hoogstein binding, or in any other sequence sensitive or specific manner. The complex may comprise two strands forming a duplex structure, three or more strands forming a multi stranded complex, a single self-hybridizing strand, or any combination of these. A hybridization reaction may constitute a step in a more extensive process, such as the initiation of a PCR, or the enzymatic cleavage of a polynucleotide by a ribozyme. A first sequence that can be stabilized via hydrogen bonding with the bases of the nucleotide residues of a second sequence can generally be “hybridizable” to the second sequence. In such a case, the second sequence can also be the to be hybridizable to the first sequence.

The terms “complement,” “complements,” “complementary,” and “complementarity,” as used herein, generally refer to a sequence that is fully complementary to and hybridizable to the given sequence. In some cases, a first sequence that is hybridizable to a second sequence or set of second sequences is specifically or selectively hybridizable to the second sequence or set of second sequences, such that hybridization to the second sequence or set of second sequences is used. Hybridizable sequences can share a degree of sequence complementarity over all or a portion of their respective lengths, such as between 25%-100% complementarity, including at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity.

The isothermal amplification methods described herein can provide advantages over existing nucleic acid amplification methods. Non-limiting examples of isothermal nucleic acid amplification methods can include helicase-dependent amplification, nicking enzyme amplification, recombinase polymerase amplification, loop-mediated isothermal amplification, and nucleic acid sequence based amplification.

The methods described herein may take advantage of DNA polymerases with high strand-displacement activity and specially designed primer sets to exponentially amplify a target sequence. The methods provided herein may provide a faster time to amplify a target nucleic acid molecule compared to a time with an existing nucleic acid amplification method. The nucleic acid target processed (e.g., nicked or cut mediated by the guide complex or enzyme) by the methods described herein may be used as an initial template to be used with any existing isothermal amplification. Different existing isothermal amplification methods can utilize different DNA polymerases. Loop-mediated isothermal amplification (LAMP) utilizes two sets of specially designed primers, termed inner and outer primers and may be performed under a constant temperature of 50-65° C. (122-149° F.). A limitation of LAMP can be use of non-specific detection methods, which may result in detection of false positives. Helicase-dependent amplification (HDA) utilizes DNA helicase activity to separate complementary strands of double strand DNA molecules, and thus may avoid temperature cycling to produce single-stranded templates for primer hybridization and subsequent primer extension by a DNA polymerase. The rolling circle amplification (RCA) method utilizes the continuous amplification of a circular DNA template by a strand-displacing DNA polymerase. RCA functions at a constant temperature (e.g., between 37° C.-42° C., [98.6-107.6° F.]) to produce a long single-stranded DNA molecule with tandem repeats of the circular template. Limitations of RCA may include challenges in mass production of target molecules, purification, and storage. Multiple displacement amplification (MDA) may utilize random exonuclease-resistant primers as well as a q29 DNA polymerase with strand-displacement activity to produce target DNA strands at a constant temperature, e.g., 30° C. (86° F.). MDA may also be used for whole genome amplification. The recombinase polymerase amplification (RPA) method is a low temperature (e.g., 37° C. [98.6° F.]) isothermal amplification that couples isothermal recombinase-driven primer targeting of a target molecule with strand-displacement DNA activity. RPA utilizes nucleoprotein complexes formed by oligonucleotide primers and recombinase proteins to guide and facilitate binding to a target DNA strand. Nucleic acid sequence-based amplification (NASBA) is an isothermal, transcription-based amplification method designed for the amplification of single-stranded RNA or DNA sequence and performed at a constant temperature of 41° C. (105.8° F.).

### Example Embodiments

The present disclosure provides methods and compositions for processing nucleic acid molecules comprising target sequences. In some aspects, the present disclosure provides for a method of processing a single-stranded nucleic acid molecule comprising a target sequence. The method can comprise contacting the single-stranded nucleic acid molecule with a guide complex comprising a guide polynucleotide under conditions where the guide polynucleotide hybridizes to the single-stranded nucleic acid molecule, wherein the guide polynucleotide comprises: (i) a non-target binding region comprising a restriction endonuclease recognition sequence for an enzyme (e.g., a restriction enzyme). The restriction enzyme can be a type IIs restriction enzyme. The guide polynucleotide can further comprise (ii) a target binding region configured to hybridize to the target sequence. The guide polynucleotide can further comprise (iii) a blocked 3′ end non-extendable by a polymerase. In some embodiments, the guide polynucleotide further comprises (i), (ii), and (iii) in 5′ to 3′ order. The non-target binding region can be located at the 5′ end of the guide polynucleotide. The target binding region can be located at the 3′ end of the guide polynucleotide. In some embodiments, the non-target binding region further comprises a sequence containing a reverse complement of the restriction endonuclease recognition sequence for the type IIs restriction enzyme 3′ to the restriction endonuclease recognition sequence for a type IIs restriction enzyme and 5′ to the target binding region configured to hybridize to the target sequence. In some embodiments, in (b) the cut exposes an extendable 3′ end of the target sequence. In some embodiments, the method further comprises reverse-transcribing the single-stranded nucleic acid molecule from an RNA.

The guide polynucleotide provided herein can be a forward guide polynucleotide (e.g., Forward Guide Oligo) configured for processing the target nucleic acid molecule in a reaction. The reaction can further comprise a reverse guide polynucleotide (e.g., Reverse Guide Oligo) configured for processing the target nucleic acid molecule or a reverse complement of the target nucleic acid molecule in the reaction.

Conditions where the guide polynucleotide hybridizes to the single-stranded nucleic acid molecule can be determined empirically or calculated based off of chemical composition of the guide polynucleotide. A variety of tools (e.g., http://www.oligoevaluator.com/LoginServlet) are available for calculating annealing/hybridization temperatures and conditions given specific sequences of polynucleotides.

The target binding region can be of a length sufficient to hybridize to the target site under conditions desirable for the assay (e.g., temperature, pH, ionic strength). In some embodiments, the target binding region is at least about 12 to about 25 nucleotides in length, including 12, 13, 14, 15, 16, 17, 18, 19, 20, 21, 22, 23, 24, and 25 nucleotides. In some embodiments, the target binding region is at least about 12 to about 30 nucleotides in length. In some embodiments, the target binding region is at least about 10 to about 25 nucleotides in length. In some embodiments, the target binding region is at least about 15 to about 25 nucleotides in length. In some embodiments, the target binding region is at least about 10 to about 30 nucleotides in length. In some embodiments, the target binding region is at least about 15 to about 30 nucleotides in length. In some embodiments, the target binding region is at least about 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides in length. In some embodiments, the target binding region is at most about 50, 45, 40, 35, 30, 25, 24, 23, 22, 21, 20, 19, 18, 17, 16, 15, 14, 13, 12, 11, 10, 9, 8, 7, 6, 5 or fewer nucleotides in length.

The enzyme described herein can comprise a type IIs restriction enzyme. The type IIs restriction enzyme can comprise one or more enzymes selected from the group consisting of BsmAI, Nt.BsmAI, Transcription Activator-Like Effector Nucleases, N.Bst9 I, N.BspD6I, Nt.BspQI, Nb.BbvCI, Nb.BsmI, Nb.BssSI, Nb.BsrDI, Nb.BtsI, Nt.AlwI, Nt.BbvCI, N.BstNBI, Nt.CviPII, Nb.Mva1269I, Nb.Bpu10I, Nt.Bpu10I, and any combinations thereof. The type IIs restriction enzyme can comprise type IIs nickases such as N.BstNBI, N.BspD6I, N.Bst9 I and Nt.BstNBI, Nt.BsmAI, BfuAI, BsmAI, BsrDI, BtsIMutI, or any combination thereof. Alternatively, the type IIs restriction enzyme can comprise BfuAI, BsmAI, BsrDI, or BtsIMutI. Additional examples of Type IIS restriction enzymes can be found at www.neb.com/tools-and-resources/selection-charts/type-iis-restriction-enzymes, which is herein incorporated by reference.

In some embodiments, the type IIs restriction enzyme comprises an engineered type IIs restriction enzyme that has a nuclease-inactivating mutation in one of its two subunits to create a nickase from an enzyme that is not naturally a nickase. In some embodiments, the type IIs restriction enzyme comprises an engineered type IIs restriction enzyme that has a mutation in one of its two subunits that create different rates of enzymatic activity of cutting one strand over the opposite strand. In some cases, the enzyme comprises two enzymes with different activities or activity rates. In some cases, the enzyme can comprise a subunit of a type IIs restriction enzyme. In some cases, the enzyme can comprise a subunit of a nicking enzyme. In some cases, the enzyme can comprise an activity for introducing a cut on the target nucleic acid sequence. For example, the enzyme can be N.BspD6I. In some cases, the enzyme can comprise an activity for introducing a cut on the complementary strand of the target nucleic acid sequence. In some cases, the enzyme can comprise an activity for introducing a cut on the guide polynucleotide (e.g., the target binding region of the guide polynucleotide). For example, the enzyme can be Nt.BstNBI.

The blocked 3′ end can comprise essentially any 3′ chemical structure that prevents extension of the guide polynucleotide by a DNA polymerase. Such structures include, but not limited to, 3′ phosphate, 3′ thiophosphate, 3′-O-methyl, a PNA, a modified base, a ddNTP, a solid support, or a spacer.

In some cases, the guide polynucleotide can further comprise an additional non-target binding region located at the 3′ end of the guide polynucleotide. The additional non-target binding region can comprise an additional site for binding to an enzyme. For example, the additional non-target binding region can comprise an additional restriction endonuclease recognition sequence for binding to a restriction enzyme. The enzyme recruited by the additional non-target binding region can be the same or different from the enzyme that is recruited by the non-target binding region of located at the 5′ end of the guide polynucleotide. The additional non-target binding region can function as a blocker to block extension of the 3′ end of the guide polynucleotide.

The method of processing the single-stranded nucleic acid molecule can further comprise introducing the type IIs restriction enzyme under conditions sufficient to cause the type IIs restriction enzyme to bind the restriction endonuclease recognition sequence and cut within the target sequence. Optimal temperatures for specific type IIs restriction enzymes can be found in e.g. the Rebase database (accessible at http://rebase.neb.com/rebase/rebase.html).

The method of processing the single-stranded nucleic acid molecule can further comprise extending the extendable 3′ end using a polymerase. In some embodiments, the polymerase is a DNA polymerase. In some embodiments, the polymerase is a DNA-dependent DNA polymerase. In some embodiments, the polymerase comprises a strand-displacing DNA polymerase. In some embodiments, the polymerase comprises a large fragment of Bacillus stearothermophilus polymerase, an exo-Klenow polymerase, a Bst 2.0 polymerase, a phi29 DNA polymerase, a T7 exo-polymerase, an OmniTaq 2 LA DNA polymerase, or any combination thereof. Such methods can further comprise adding other factors alongside the polymerase sufficient to add nucleotides to the 3′ end, including dNTPs, appropriate buffering agents, and cofactors (e.g., divalent cations). The dNTPs may be natural or unnatural dNTPs. The natural dNTPs can comprise dATP, dCTP, dGTP, dTTP, and/or dUTP. The unnatural dNTPs can be α-thiol dNTPs (e.g., S-dNTPs). S-dNTPS can comprise dATPαS, dCTPαS, dGTPαS, and/or dTTPαS.

The target sequence processed by the methods provided herein can be used for further downstream applications, e.g., isothermal amplifications. In some cases, the reagents for carrying out the amplification can be in the same mixture as the reagents for target processing. In some aspects, the present disclosure provides for a method of amplifying a single-stranded nucleic acid molecule comprising a target sequence, the method comprising: (a) contacting the single-stranded nucleic acid molecule with a guide complex comprising a guide polynucleotide under conditions where the guide polynucleotide hybridizes to the single-stranded nucleic acid molecule, wherein the guide polynucleotide comprises: (i) a non-target binding region comprising a restriction endonuclease recognition sequence for a type IIs restriction enzyme, (ii) a target binding region configured to hybridize to the target sequence, and (iii) a blocked 3′ end non-extendable by a polymerase; (b) introducing the type IIs restriction enzyme under conditions sufficient to cause the type IIs restriction enzyme to bind the restriction endonuclease recognition sequence and cut within the target sequence to generate an extendable 3′ end; and (c) extending the extendable 3′ end of the target sequence using a polymerase. In some embodiments, the guide polynucleotide further comprises (i), (ii), and (iii) in 5′ to 3′ order. In some embodiments, the non-target binding region further comprises a sequence containing a reverse complement of the restriction endonuclease recognition sequence for the type IIs restriction enzyme 3′ to the restriction endonuclease recognition sequence for a type IIs restriction enzyme and 5′ to the target binding region configured to hybridize to the target sequence. In some embodiments, the guide polynucleotide is a first guide polynucleotide, and the guide complex comprises a second guide polynucleotide, wherein the second guide polynucleotide comprises (i) a non-target binding region that is complementary with the non-target binding region of the first guide polynucleotide and (ii) a target binding region configured to hybridize to the target sequence. In some cases, when the first guide polynucleotide of the guide complex is hybridized to the target polynucleotide sequence, the target binding region of the second guide polynucleotide of the guide complex is not hybridized to the target sequence. In some embodiments, the first guide polynucleotide and the second guide polynucleotide of the guide complex hybridize to form a dimer. In some embodiments, the first guide polynucleotide and the second guide polynucleotide of the guide complex hybridize at a common 5′ region. In some embodiments, the first guide polynucleotide and the second guide polynucleotide hybridize via the non-target binding region of the first guide polynucleotide and the second guide polynucleotide to form the dimer having a double-stranded binding region. In some embodiments, the double-stranded binding region comprises the restriction endonuclease recognition sequence. In some embodiments, the type IIs restriction enzyme binds to the double-stranded binding region of the dimer. A forward guide polynucleotide (or complex) can comprise one or more guide polynucleotides including the first guide polynucleotide and the second guide polynucleotide described herein. The first guide polynucleotide and the second guide polynucleotide can be homodimer or heterodimer. For example, the non-target binding region at the 5′ end of the first guide polynucleotide and the non-target binding region at the 5′ end of the second guide polynucleotide can comprise the same sequence (e.g., a palindromic sequence), and the target binding region at the 3′ end of the first or the second guide polynucleotide can be different. In some embodiments, a target binding region can be configured to hybridize to a target sequence. Alternatively, a target binding region can be configured to hybridize to a different target sequence.

In some cases, a reverse guide polynucleotide (or complex) can comprise a plurality of guide polynucleotides including the first guide polynucleotide and the second guide polynucleotide. In some cases, a reverse guide polynucleotide and a forward guide polynucleotide can comprise a same sequence (e.g., a palindromic sequence) at the 5′ end such that the reverse guide polynucleotide and the forward guide polynucleotide can hybridize to form a heterodimer. The target binding region of the forward guide polynucleotide and the target binding region of the reverse guide polynucleotide can comprise different sequences.

In some aspects, the present disclosure provides for a method of amplifying a single-stranded nucleic acid molecule comprising a target sequence, the method comprising: (a) contacting a guide complex with the single-stranded nucleic acid molecule, wherein the guide complex comprises: (i) a first guide polynucleotide comprising, from 5′ to 3′, a non-target binding region and a target binding region that hybridizes with the target sequence of the single-stranded nucleic acid molecule, and (i) a second guide polynucleotide that hybridizes with the non-target binding region of the first guide molecule to form a double-stranded binding region, wherein the double-stranded binding region binds to an enzyme; and (b) cutting the target sequence using the enzyme to expose an extendable 3′ end of the target sequence. In some cases, an extendable 3′ end is a 3′ hydroxyl group. In some embodiments, if a target molecule is an RNA, the method can further comprise reverse-transcribing, prior to contacting the target molecule with the guide complex, the single-stranded nucleic acid molecule from the RNA. For example, the target RNA molecule can be reverse transcribed using a reverse transcriptase to generate a DNA molecule, which can be subject to further processing using the methods described herein. The DNA molecule can be a single-stranded DNA molecule (ssDNA). In some cases, a reverse transcription reaction can be used to make a ssDNA target from an initial RNA target. In some cases, a reverse transcription reaction can comprise a reverse transcriptase and a reverse transcription primer. The reverse transcriptase can comprise avian myeloblastosis virus (AMV) reverse transcriptase (RT), Moloney murine leukemia virus RT (M-MLV RT), telomerase RT, or human immunodeficiency virus type 1 RT (HIV-1 RT).

In some cases, the method of amplifying the single-stranded nucleic acid molecule comprising the target sequence further comprises extending the extendable 3′ end of the target sequence with a polymerase to generate an extension product, wherein the extension product displaces the second guide polynucleotide. In some cases the polymerase extension creates a double-stranded product displacing the second guide polynucleotide. In some embodiments, the extending comprises incubation in the presence of a DNA polymerase such as strand-displacing DNA polymerase, including any of the strand-displacing polymerases described herein. The extending can also comprise incubation in the presence of factors alongside the polymerase sufficient to add nucleotides to the 3′ end, including dNTPs, appropriate buffering agents, and cofactors (e.g. divalent cations). The dNTPs may be natural or unnatural dNTPs. The natural dNTPs can comprise dATP, dCTP, dGTP, dTTP, and/or dUTP. The unnatural dNTPs can be α-thiol dNTPs (e.g., S-dNTPs). S-dNTPS can comprise dATPαS, dCTPαS, dGTPαS, and/or dTTPαS.

In some cases, the method of amplifying the single-stranded nucleic acid molecule comprising the target sequence further comprises cutting the first guide polynucleotide within the target binding region to expose an extendable 3′ end of the first guide polynucleotide. In some embodiments the cutting can comprise introducing a type IIs restriction enzyme under conditions sufficient to cause the type IIs restriction enzyme to bind the restriction endonuclease recognition sequence and cut the first guide polynucleotide within the target binding region. In some embodiments, the extendable 3′ end comprises a 3′ hydroxyl.

In some cases, the method of amplifying the single-stranded nucleic acid molecule comprising the target sequence further comprises extending the extendable 3′ end of the first guide polynucleotide using a polymerase to generate a complementary molecule of the target sequence of the single-stranded nucleic acid molecule, thereby amplifying the single-stranded nucleic acid molecule. The polymerase can be strand-displacing DNA polymerase, including any of the strand-displacing polymerases described herein. The extending can also comprise incubation in the presence of factors alongside the polymerase sufficient to add nucleotides to the 3′ end, including dNTPs, appropriate buffering agents, and cofactors (e.g., divalent cations). The dNTPs may be natural or unnatural dNTPs. The natural dNTPs can comprise dATP, dCTP, dGTP, dTTP, and/or dUTP. The unnatural dNTPs can be α-thiol dNTPs (e.g., S-dNTPs). S-dNTPS can comprise dATPαS, dCTPαS, dGTPαS, and/or dTTPαS.

In some embodiments, the second guide polynucleotide in the method of amplifying a single-stranded nucleic acid molecule comprising a target sequence comprises, from 5′ to 3′ (i) a non-target binding region that hybridizes with the non-target binding region of the first guide polynucleotide and (ii) a target binding region configured to hybridize with the target sequence. In some embodiments, the method further comprises prior to (b), cutting the first guide polynucleotide within the target binding region using the enzyme, wherein the guide complex dissociates from the single-stranded nucleic acid molecule. In some embodiments, the method further comprises cutting the first guide polynucleotide within the target binding region to expose an extendable 3′ end of the first guide polynucleotide and extending the extendable 3′ end of the first guide polynucleotide using a polymerase to generate a complementary molecule of the target sequence of the single-stranded nucleic acid molecule repeatedly to generate a plurality of complementary molecules of the target sequence of the single-stranded nucleic acid molecule. In some embodiments, an additional guide complex binds to the complementary molecule. In some embodiments, the method further comprises using the complementary molecule with the additional guide complex bound thereto as a starting template to generate copies of the target molecule. In some embodiments, the enzyme is a type IIs restriction enzyme. In some embodiments, the type IIs restriction enzyme comprises N.BstNBI, N.Bst9 I and N.BspD6I, Nt.BsmAI, BfuAI, BsmAI, BsrDI, BtsIMutI, BfuAI, BsmAI, BsrDI, BtsIMutI, a functional fragment thereof, or a combination thereof. In some embodiments, the guide polynucleotide comprises a blocked 3′ end non-extendable by a polymerase. The blocked 3′ end can comprise essentially any 3′ chemical structure that prevents extension of the guide polynucleotide by a DNA polymerase, including any structures with such activity described herein. In some embodiments, the blocked 3′ end comprises a PNA, a modified base, a phosphate group, a ddNTP, a solid support, or a spacer. In some embodiments, the single-stranded nucleic acid molecule with the cut and the guide polynucleotide bound thereto is used as a starting template for an amplification. In some embodiments, the amplification is an isothermal amplification. In some embodiments, the enzyme comprises asymmetric propensity to cleave one strand of a DNA duplex. In some embodiments, the enzyme exhibits a high-frequency endonuclease activity. In some embodiments, the high-frequency endonuclease activity is from a large subunit of the enzyme. In some embodiments, the enzyme exhibits a low-frequency endonuclease activity. In some embodiments, the low-frequency endonuclease activity is from a small subunit of the enzyme. In some embodiments, the enzyme exhibits at least two differential enzymatic activity rates. In some embodiments, the at least two differential enzymatic activity rates comprise two differential endonuclease activity rates when cutting two different cutting sites. In some embodiments, one of the two differential endonuclease activity rates comprises cutting the target sequence of the single-stranded nucleic acid molecule with low frequency. In some embodiments, one of the two differential endonuclease activity rates comprises cutting the target binding region of the guide polynucleotide by with high frequency. In some embodiments, the two differential endonuclease activity rates are asymmetric or not equal. In some embodiments, the enzyme comprises N.BstNBI, N.Bst9 I and N.BspD6I, Nt.BsmAI, BfuAI, BsmAI, BsrDI, BtsIMutI, BfuAI, BsmAI, BsrDI, BtsIMutI, or a combination thereof.

In some embodiments, a temperature is changed over the course of the method. In some embodiments, a first activity rate of the at least two differential enzymatic activity rates is favored at a first temperature, and a second activity rate of the at least two differential enzymatic activity rates is favored at a second temperature different from the first temperature. In some embodiments, a first temperature wherein a first enzymatic activity rate is favored can be about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments, a first temperature wherein a first enzymatic activity rate is favored is between about 15° C.-50° C., between about 20° C.-45° C., between about 30° C.-45° C., between about 30° C.-40° C., or between about 32° C.-39° C. In some embodiments, a second temperature wherein a second enzymatic activity rate is favored can be about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. In some embodiments, a second temperature wherein a second enzymatic activity rate is favored is between about 45° C.-80° C., between about 50° C.-80° C., between about 50° C.-70° C., between about 50° C.-60° C., between about 52° C.-58° C.

In some embodiments, a temperature may be changed over the course of the method for a period of time. The period of time at which a temperature is changed may benefit the enzymatic activity rate during the reaction. A temperature change can comprise a first temperature or a second temperature. In some embodiments, a first temperature change or a second temperature change may occur over a duration of time of at least about 15 seconds, at least about 30 seconds, at least about 1 minute, at least about 1.5 minutes, at least about 2 minutes, at least about 2.5 minutes, at least about 3 minutes, at least about 3.5 minutes, at least about 4 minutes, at least about 4.5 minutes, at least about 5 minutes, at least about 5.5 minutes, at least about 6 minutes, at least about 6.5 minutes, at least about 7 minutes, at least about 8 minutes, at least about 9 minutes, at least about 10 minutes, at least about 12 minutes, or at least about 15 minutes. In some embodiments, a first temperature change or a second temperature change may occur over a duration of time of at most about 15 minutes, at most about 12 minutes, at most about 10 minutes, at most about 9 minutes, at most about 8 minutes, at most about 7 minutes, at most about 6.5 minutes, at most about 6 minutes, at most about 5.5 minutes, at most about 5 minutes, at most about 4.5 minutes, at most about 4 minutes, at most about 3.5 minutes, at most about 3 minutes, at most about 2.5 minutes, at most about 2 minutes, at most about 1.5 minutes, at most about 1 minute, at most about 30 seconds, or at most about 15 seconds.

In some embodiments, a first temperature change or a second temperature change may occur over a duration of time from about 1 minute to about 15 minutes. In some embodiments, the sample may be heated from a range from about 1 minute to about 2 minutes, about 1 minute to about 2.5 minutes, about 1 minute to about 3 minutes, about 1 minute to about 3.5 minutes, about 1 minute to about 4 minutes, about 1 minute to about 5 minutes, about 1 minute to about 6 minutes, about 1 minute to about 7 minutes, about 1 minute to about 7.5 minutes, about 1 minute to about 10 minutes, about 1 minute to about 15 minutes, about 2 minutes to about 2.5 minutes, about 2 minutes to about 3 minutes, about 2 minutes to about 3.5 minutes, about 2 minutes to about 4 minutes, about 2 minutes to about 5 minutes, about 2 minutes to about 6 minutes, about 2 minutes to about 7 minutes, about 2 minutes to about 7.5 minutes, about 2 minutes to about 10 minutes, about 2 minutes to about 15 minutes, about 2.5 minutes to about 3 minutes, about 2.5 minutes to about 3.5 minutes, about 2.5 minutes to about 4 minutes, about 2.5 minutes to about 5 minutes, about 2.5 minutes to about 6 minutes, about 2.5 minutes to about 7 minutes, about 2.5 minutes to about 7.5 minutes, about 2.5 minutes to about 10 minutes, about 2.5 minutes to about 15 minutes, about 3 minutes to about 3.5 minutes, about 3 minutes to about 4 minutes, about 3 minutes to about 5 minutes, about 3 minutes to about 6 minutes, about 3 minutes to about 7 minutes, about 3 minutes to about 7.5 minutes, about 3 minutes to about 10 minutes, about 3 minutes to about 15 minutes, about 3.5 minutes to about 4 minutes, about 3.5 minutes to about 5 minutes, about 3.5 minutes to about 6 minutes, about 3.5 minutes to about 7 minutes, about 3.5 minutes to about 7.5 minutes, about 3.5 minutes to about 10 minutes, about 3.5 minutes to about 15 minutes, about 4 minutes to about 5 minutes, about 4 minutes to about 6 minutes, about 4 minutes to about 7 minutes, about 4 minutes to about 7.5 minutes, about 4 minutes to about 10 minutes, about 4 minutes to about 15 minutes, about 5 minutes to about 6 minutes, about 5 minutes to about 7 minutes, about 5 minutes to about 7.5 minutes, about 5 minutes to about 10 minutes, about 5 minutes to about 15 minutes, about 6 minutes to about 7 minutes, about 6 minutes to about 7.5 minutes, about 6 minutes to about 10 minutes, about 6 minutes to about 15 minutes, about 7 minutes to about 7.5 minutes, about 7 minutes to about 10 minutes, about 7 minutes to about 15 minutes, about 7.5 minutes to about 10 minutes, about 7.5 minutes to about 15 minutes, or about 10 minutes to about 15 minutes.

In some embodiments, the enzyme comprises two different active sites or endonuclease domains conferring the at least two differential enzymatic activities. In some embodiments, the target sequence comprises a recognition site specifically recognized by the enzyme or a first activity of the at least two differential enzymatic activities of the enzyme to introduce a cut. In some embodiments, the target binding region of the guide polynucleotide comprises a recognition site specifically recognized by the enzyme or a second activity of the at least two differential enzymatic activities of the enzyme to introduce a cut. The target binding region can be of a length sufficient to hybridize to the target site under conditions desirable for the assay (e.g., temperature, pH, ionic strength). In some embodiments, the target binding region is at least about 10, 11, 12, 13, 14, 15, 16, 17, 18, 19, 20, 21, 22, 23, 24, 25, 26, 27, 28, 29, 30, 35, 40, 45, 50 or more nucleotides in length. In some embodiments, the target binding region is at most about 50, 45, 40, 35, 30, 25, 24, 23, 22, 21, 20, 19, 18, 17, 16, 15, 14, 13, 12, 11, 10 or less nucleotides in length. In some embodiments, the target binding region is at least about 15 to about 25 nucleotides in length, including 15, 16, 17, 18, 19, 20, 21, 22, 23, 24, and 25 nucleotides. In some embodiments, the target binding region is at least about 15 to about 25 nucleotides in length. In some embodiments, the target binding region is at least about 10 to about 25 nucleotides in length. In some embodiments, the target binding region is at least about 12 to about 25 nucleotides in length.

In some embodiments, a concentration of the guide polynucleotide is at least about 0.1 μM, at least about 1 μM, or about 0.1 μM to about 4 μM. In some embodiments, a concentration of the guide polynucleotide is at least about 0.1 μM, 0.2 M, 0.3 μM, 0.4 μM, 0.5 μM, 0.6 μM, 0.7 μM, 0.8 μM, 0.9 μM, 1.0 μM, 1.5 μM, 2.0 μM, 2.5 μM, 3.0 μM, 3.5 M, 4 μM or more. In some embodiments, the non-target binding region comprises a palindromic sequence. In some embodiments, the non-target binding region is self-complementary or forms a self-annealing dimer under reaction conditions. In some embodiments, the non-target binding region is at least about 10, 11, 12, 13, 14, 15, 16, 17, 18, 19, 20, 21, 22, 23, 24, 25 or more nucleotides in length. In some embodiments, the non-target binding region is at most about 25, 24, 23, 22, 21, 20, 19, 18, 17, 16, 15, 14, 13, 12, 11 or less nucleotides in length. In some embodiments, the single-stranded nucleic acid molecule is a single-stranded deoxyribonucleic acid (ssDNA) or a single-stranded ribonucleic acid (ssRNA). In some embodiments, the method further comprises reverse-transcribing the single-stranded nucleic acid molecule from an RNA. In some embodiments, the target binding region comprises at least one peptide nucleic acid (PNA) residue. In some embodiments, the polymerase has strand displacement activity.

In some embodiments, the methods described herein may result in a faster amplification result compared to nucleic acid amplification protocols without the programmed restriction enzyme. A metric of speed of an amplification may be a cycle threshold. A “cycle threshold” can comprise a number of cycles needed for a signal (e.g., fluorescent signal) to exceed a background threshold level. A lower cycle threshold value can indicate a greater amount of target nucleic acid in a sample. In some embodiments, a nucleic acid amplification using the methods described herein can result in a lower cycle threshold compared to loop-mediated isothermal amplification (LAMP), helicase-dependent amplification (HDA), rolling circle amplification (RCA), or other amplification methods known in the art. A cycle threshold for a sample processing method described herein may be at least about 2%, at least about 5%, at least about 8%, at least about 10%, at least about 12%, at least about 15%, at least 18%, at least about 20%, at least about 25%, at least about 30%, at least about 40%, at least about 50%, or at least about 60% less than a cycle threshold for LAMP. A cycle threshold for a sample processing method described herein may be at most about 60%, at most about 50%, at most about 40%, at most about 30%, at most about 25%, at most about 20%, at most about 18%, at most about 15%, at most about 12%, at most about 10%, at most about 8%, at most about 5%, or at most about 2% less than a cycle threshold for LAMP. A cycle threshold for a sample processing method described herein may be from about 1% to about 50% less than a cycle threshold for LAMP. A cycle threshold for a sample processing method described herein may be from about 1% to about 2%, about 1% to about 3%, about 1% to about 4%, about 1% to about 5%, about 1% to about 8%, about 1% to about 10%, about 1% to about 12%, about 1% to about 15%, about 1% to about 20%, about 1% to about 25%, about 1% to about 50%, about 2% to about 3%, about 2% to about 4%, about 2% to about 5%, about 2% to about 8%, about 2% to about 10%, about 2% to about 12%, about 2% to about 15%, about 2% to about 20%, about 2% to about 25%, about 2% to about 50%, about 3% to about 4%, about 3% to about 5%, about 3% to about 8%, about 3% to about 10%, about 3% to about 12%, about 3% to about 15%, about 3% to about 20%, about 3% to about 25%, about 3% to about 50%, about 4% to about 5%, about 4% to about 8%, about 4% to about 10%, about 4% to about 12%, about 4% to about 15%, about 4% to about 20%, about 4% to about 25%, about 4% to about 50%, about 5% to about 8%, about 5% to about 10%, about 5% to about 12%, about 5% to about 15%, about 5% to about 20%, about 5% to about 25%, about 5% to about 50%, about 8% to about 10%, about 8% to about 12%, about 8% to about 15%, about 8% to about 20%, about 8% to about 25%, about 8% to about 50%, about 10% to about 12%, about 10% to about 15%, about 10% to about 20%, about 10% to about 25%, about 10% to about 50%, about 12% to about 15%, about 12% to about 20%, about 12% to about 25%, about 12% to about 50%, about 15% to about 20%, about 15% to about 25%, about 15% to about 50%, about 20% to about 25%, about 20% to about 50%, or about 25% to about 50% less than a cycle threshold for LAMP.

In some embodiments, a cycle threshold value for a sample processing method described herein may be at least about 1, at least about 2, at least about 3, at least about 4, at least about 5, at least about 6, at least about 7, at least about 8, at least about 9, at least about 10, at least about 12, at least about 15, at least about 18, at least about 20, at least about 25, at least about 30, at least about 35, or at least about 40. In some embodiments, a cycle threshold value for a sample processing method described herein may be at most about 40, at most about 35, at most about 30, at most about 25, at most about 20, at most about 18, at most about 15, at most about 12, at most about 10, at most about 9, at most about 8, at most about 7, at most about 6, at most about 5, at most about 4, at most about 3, at most about 2, or at most about 1.

In some aspects, the present disclosure provides for a polynucleotide-polypeptide complex comprising: a single-stranded nucleic acid molecule having bound thereto a guide complex, wherein the guide complex comprises: a first guide polynucleotide comprising, from 5′ to 3′, a non-target binding region and a target binding region that hybridizes with a target sequence of the single-stranded nucleic acid molecule, and a second guide polynucleotide that hybridizes with the non-target binding region of the first guide molecule to form a double-stranded binding region, wherein the double-stranded binding region comprises a restriction endonuclease recognition sequence for an enzyme that is a type IIs restriction enzyme.

In some aspects, the present disclosure provides for a system of processing a single-stranded nucleic acid molecule comprising a target sequence, the system comprising: the single-stranded nucleic acid molecule having bound thereto a guide complex comprising a guide polynucleotide, wherein the guide polynucleotide comprises: (i) a non-target binding region comprising a restriction endonuclease recognition sequence for an enzyme that is a type IIs restriction enzyme, (ii) a target binding region configured to hybridize to the target sequence, and (iii) a blocked 3′ end non-extendable by a polymerase; and the enzyme bound to the restriction endonuclease recognition sequence of the non-target binding region.

The methods, systems, or kits provided herein can be used to process or analyze one sample or one target nucleic acid molecule or target sequence. Alternatively, the methods, systems or kits provided herein can be used to process or analyze two or more different samples, or two or more different target nucleic acid molecules or target sequences in a same reaction mixture (e.g., a single reaction). For example, the methods, systems or kits provided herein can be used to process or analyze 2, 3, 4, 5, 6, 7, 8, 9, 10, 11, 12, 13, 14, 15, 16, 17, 18, 19, 20, or more different target nucleic acid sequences in a same reaction mixture. In some embodiments, the reaction mixture is lyophilized. In some embodiments, the reaction mixture is not lyophilized.

In various embodiments, the guide polynucleotide comprises a target binding region. The sequence of the target binding region can be designed according to the target sequence by following similar rules for primer design. For example, primer design can be based on various parameters, including melting temperature of the primers (which may be calculated using the nearest neighbor algorithm shown in John Santa Lucia, Jr., “A unified view of polymers, dumbbell, and oligonucleotide DNA nearest-neighbor thermal dynamics,” Proc. Natl. Acad. Sci. USA., Vol. 95, 1460-1465(1998) (the contents of which are incorporated herein by reference in their entirety)), primer composition (e.g., nucleotide composition such as GC content may be determined and filtered using software and penalized, as is the composition of the GC content of the hairpin, 3′ end of the primer, and the specific parameters that may be evaluated are the homopolymer nucleotides in length, hairpin formation, GC content and amplicon size), predicted dimer-dimer formations, average extension length and the like. In the case of multiplexed reactions with two or more target sequences, the target binding region (or primer) can be designed to minimize cross-reactivity. The non-target binding region of the guide polynucleotide can be designed to be non-hybridizable with the target sequence and contain a sequence that can be recognized by an enzyme (e.g., the restriction enzyme) described herein.

In some aspects, the present disclosure provides for a method or a system of multiplexing the processing of more than one nucleic acid molecules, each nucleic acid molecule comprising a different target sequence. The method or system can comprise, for each nucleic acid molecule comprising a different target sequence, a nucleic acid molecule having bound thereto a guide complex comprising a guide polynucleotide. The guide polynucleotide can comprise: (i) a non-target binding region comprising a restriction endonuclease recognition sequence for an enzyme that is a type IIs restriction enzyme, (ii) a target binding region configured to hybridize to the target sequence, and (iii) a blocked 3′ end non-extendable by a polymerase. The enzyme can bind to the restriction endonuclease recognition sequence of the non-target binding region. In some aspects, a multiplexed processing of one or more nucleic acid molecules comprises using two or more different sets of primers or guide complexes, each targeting a different target. In some aspects, multiplexed processing of one or more nucleic acid molecules comprises a reaction mixture comprising two more different detection probes or fluorophores, each targeting a different target sequence. Each of the two or more different detection probes can be linked to a different fluorophore for multiplexed detection.

The amplification product can be detected by various methods. The amplification products may be detected by gel electrophoresis, thus detecting reaction products having a specific length. The nucleotides may, for example, be labeled, such as, for example, with biotin. Biotin-labeled amplified sequences may be captured using avidin bound to a signal generating enzyme, for example, peroxidase. Nucleic acid detection methods may employ the use of dyes that specifically stain double-stranded DNA. Intercalating dyes that exhibit enhanced fluorescence upon binding to DNA or RNA can be used. Dyes may be, for example, DNA or RNA intercalating fluorophores and may include but are not limited to the following examples: Acridine orange, ethidium bromide, Hoechst dyes, PicoGreen, propidium iodide, SYBRI (an asymmetrical cyanine dye), SYBRII, TOTO (a thiaxole orange dimer) and YOYO (an oxazole yellow dimer), and the like. Dyes can provide an opportunity for increasing the sensitivity of nucleic acid detection when used in conjunction with various detection methods and may have varying optimal usage parameters. Nucleic acid detection methods may also employ the use of labeled nucleotides incorporated directly into the target sequence or into probes containing complementary or substantially complementary sequences to the target of interest. Such labels may be radioactive and/or fluorescent in nature. Labeled nucleotides, which can be detected but otherwise function as native nucleotides, can be to be distinguished from modified nucleotides, which do not function as native nucleotides. The production or presence of target nucleic acids and nucleic acid sequences may be detected and monitored by Molecular Beacons. The production or presence of target nucleic acids and nucleic acid sequences may also be detected and monitored by Fluorescence resonance energy transfer (FRET).

A wide range of fluorophores and/or dyes may be used in the methods described herein according to the present disclosure. Available fluorophores include coumarin; fluorescein; tetrachlorofluorescein; hexachlorofluorescein; Lucifer yellow; rhodamine; BODIPY; tetramethylrhodamine; Cy3; Cy5; Cy7; cosine; Texas red; SYBR Green I; SYBR Gold; 5-FAM (also called 5-carboxyfluorescein; also called Spiro(isobenzofuran-1 (3H), 9′-(9H) xanthene)-5-carboxylic acid, 3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl)-3,6-bis(dimethylamino); EDANS (5-((2-aminoethyl)amino) naphthalene-1-sulfonic acid); 1,5-IAEDANS (5-((((2-iodoacetyl)amino)ethyl)amino) naphthalene-1-sulfonic acid); DABCYL (4-((4-(dimethylamino)phenyl) azo)benzoic acid) Cy5 (Indodicarbocyanine-5) Cy3 (Indo-dicarbocyanine-3); BODIPY FL (2,6-dibromo-4,4-difluoro-5,7-dimethyl-4-bora-3a,4a-diaza-s-indacene-3-proprionic acid); Quasar-670 (Bioresearch Technologies); CalOrange (Bioresearch Technologies); and Rox as well as suitable derivatives thereof. Combination fluorophores such as fluorescein-rhodamine dimers may also be suitable. Fluorophores may be chosen to absorb and emit in the visible spectrum or outside the visible spectrum, such as in the ultraviolet or infrared ranges. Suitable quenchers may also include DABCYL and variants thereof, such as DABSYL, DABMI and Methyl Red. Fluorophores may also be used as quenchers, because they tend to quench fluorescence when touching certain other fluorophores. In some cases, quenchers may be chromophores such as DABCYL or malachite green, or fluorophores that may not fluoresce in the detection range when the probe is in the open conformation.

In some embodiments, at least 2, at least 3, at least 4, at least 5, at least 6 at least 7, at least 8, at least 9, at least 10, or more pluralities of single-stranded nucleic acid molecules can be processed in the same reaction. In some embodiments, each plurality of the multiplexed nucleic acid molecules is derived from a different sample.

A sample described herein can comprise a biological sample. A sample can comprise a single-stranded nucleic acid molecule. Alternatively, a sample can comprise a double-stranded nucleic acid molecule.

A sample can comprise a fluid sample. Non-limiting examples of fluid samples can include blood, plasma, urine, feces saliva, sweat, tears, pericardial fluid, peritoneal fluid, pleural fluid, cerebrospinal fluid, gastric juice, respiratory secretion, semen, synovial fluid, or amniotic fluid.

In some embodiment, the sample comprises a blood sample, a swab sample, a saliva sample, a urine sample, a cerebrospinal fluid sample, a pleural fluid sample, a rectal sample, a vaginal sample, a stool sample, a sputum sample, and/or a lymph sample for nucleic acid amplification. In some embodiments, the swab sample comprises a vaginal swab, an oral swab, and/or a rectal swab. In some embodiments, the sample is a solid sample. In some embodiments, the sample is a liquid sample. In some embodiments, the sample is obtained from a subject. In some embodiments, the subject has a disease, a condition, or an infection. In some embodiments, the sample comprises a purified sample. In some embodiments, the sample is a combination of two, three, four, five, or more types of samples. In some embodiments, the sample comprises one, two, three, four, five, six, seven, eight, nine, ten, or more target nucleic acid molecules.

A sample may be obtained invasively (e.g., tissue biopsy) or non-invasively (e.g., venipuncture). The sample may be an environmental sample. The sample may be a water sample (e.g., a water sample obtained from a lake, stream, river, estuary, bay, or ocean). The sample may be a soil sample. The sample may be a tissue or fluid sample from a subject, such as saliva, semen, blood (e.g., whole blood), serum, synovial fluid, tear, urine, or plasma. The sample may be a tissue sample, such as a skin sample or tumor sample. The sample may be obtained from a portion of an organ of a subject. The sample may be a cellular sample. The sample may be a cell-free sample (e.g., a plasma sample comprising cell-free analytes or nucleic acids). A sample may be a solid sample or a liquid sample. A sample may be a biological sample or a non-biological sample. A sample may comprise an in-vitro sample or an ex-vivo sample. Non-limiting examples of a sample include an amniotic fluid, bile, bacterial sample, breast milk, buffy coat, cells, cerebrospinal fluid, chromatin DNA, ejaculate, nucleic acids, plant-derived materials, RNA, saliva, semen, blood, serum, soil, synovial fluid, tears, tissue, urine, water, whole blood or plasma, and/or any combination and/or any fraction thereof. In one example, the sample may be a plasma sample that may comprise DNA. In another example, the sample may comprise a cell sample that may comprise cell-free DNA.

A sample may be a mammalian sample. For example, a sample may be a human sample.

Alternatively, a sample may be a non-human animal sample. Non-limiting examples of a non-human sample include a cat sample, a dog sample, a goat sample, a guinea pig sample, a hamster sample, a mouse sample, a pig sample, a non-human primate sample (e.g., a gorilla sample, an ape sample, an orangutan sample, a lemur sample, or a baboon sample), a rat sample, a sheep sample, a cow sample, and a zebrafish sample.

The sample may comprise nucleic acids (e.g., circulating and/or cell-free DNA fragments). Nucleic acids may be derived from eukaryotic cells, prokaryotic cells, or non-cellular sources (e.g., viral particles). A nucleic acid may refer to a substance whose molecules consist of many nucleotides linked in a long chain. Non-limiting examples of the nucleic acid include an artificial nucleic acid analog (e.g., a peptide nucleic acid, a morpholino oligomer, a locked nucleic acid, a glycol nucleic acid, or a threose nucleic acid), chromatin, niRNA, cDNA, DNA, single stranded DNA, double stranded DNA, genomic DNA, plasmid DNA, or RNA. A nucleic acid may be double stranded or single stranded. A sample may comprise a nucleic acid that may be intracellular. Alternatively, a sample may comprise a nucleic acid that may be extracellular (e.g., cell-free). A sample may comprise a nucleic acid (e.g., chromatin) that may be fragmented.

A sample can be obtained from a virus, a bacterium, an archaea, or a eukarya. In some embodiments, a sample is obtained from a bacterium. A bacterium can be a spherical-shaped bacterium, a rod-shaped bacterium, a spiral-shaped bacterium, a comma-shaped bacterium, or a corkscrew-shaped bacterium. Non-limiting examples of bacteria are Streptococcus pneumoniae, Streptococcus pyogenes, Legionella pneumonia, Bordetella bronchiseptica, Enterobacter aerogenes, Pasteurella multocida, Proteus mirabilis, Staphylococcus aureus, Haemophilus influenzae, Mycoplasma pneumoniae, Klebsiella pneumoniae, Escherichia coli, Pseudomonas aeruginosa, Trichomonas vaginalis, Neisseria gonorrhoeae, Chlamydia pneumoniae and Chlamydia trachomatis. In some embodiments, a sample is obtained from a virus. A virus can be a double-stranded DNA virus, a single-stranded DNA virus, a double-stranded RNA virus, a single-stranded RNA virus, a positive sense single-stranded reverse transcriptase virus, or a double-stranded DNA reverse transcriptase virus. In some cases, the sample comprises a human gene such as RPP30. In some embodiments, sample preparation can comprise extracting nucleic acids from a sample. In some embodiments, sample preparation can comprise extracting nucleic acids from a sample by heating the sample. For example, a target nucleic acid (e.g., target RNA, target DNA) may be extracted or released from a biological sample during heating phases of nucleic acid amplification. Alternatively or in addition to the heating, a target nucleic acid (e.g., target RNA, target DNA) may be extracted or released from a biological sample using a cartridge system wherein a sample can be mixed with a lysis buffer and then drawn through a filter thereby capturing the target nucleic acid in the filter. In some cases, a cartridge system can also comprise washing steps to remove contaminants. An elution buffer can be added to the cartridge to remove the target nucleic acid from the filter for further processing or analysis. The cartridge system can be an automated cartridge system. In some cases, the cartridge system can be the M1 Sample Prep® Cartridge Kit (SKU: 3000536, Biomeme, Inc.). In some cases, the sample preparation method described herein can use the cartridge system for automated sample processing. Details of the sample preparation cartridge and related methods is described in the U.S. application Ser. No. 16/817,733, the entire content of which is incorporated herein by reference. It is to be understood that the sample described herein can be processed by various other methods or any commercially available nucleic acid extraction kits or methods.

In some aspects, the present disclosure provides for a kit comprising any of the guide complexes or any of the guide polynucleotides described herein. In some embodiments, the kit further comprises a probe or a dye for detecting an amplification product generated using the kit. In some embodiments, the kit further comprises an informational material describing an instruction of using the kit. In some embodiments, the information comprises optimal reaction temperatures for amplification using the guide complexes or the guide polynucleotides, or optimal buffer conditions for the same. In some embodiments, the kit further comprises a type II restriction enzyme compatible with the guide polynucleotides or guide complexes as described herein. In some embodiments, the kit further comprises a strand-displacing polymerase. The kits can be compartmentalized for ease of use and can include one or more containers with reagents. In some embodiments, all of the kit components are packaged together. Alternatively, one or more individual components of the kit can be provided in a separate package from the other kits components.

### Computer Systems

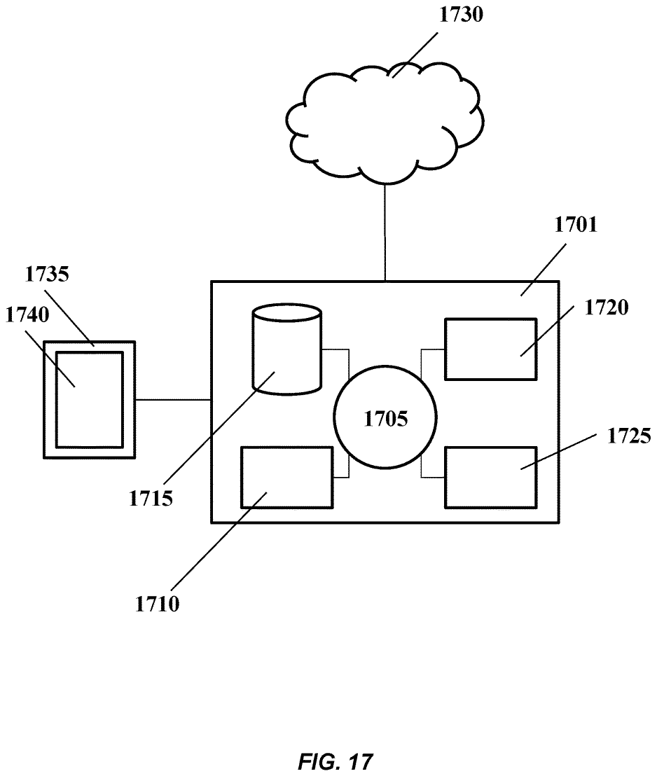

The present disclosure provides computer systems that are programmed to implement methods of the disclosure. FIG. 17 shows a computer system 1701 that can be programmed or otherwise configured to analyze polynucleotide-polypeptide complexes. Alternatively or in addition to, the computer system 1701 can be programmed or otherwise configured to analyze single-stranded nucleic acid molecule processing data. The computer system 1701 can be an electronic device of a user or a computer system that is remotely located with respect to the electronic device. The electronic device can be a mobile electronic device.

The computer system 1701 includes a central processing unit (CPU, also “processor” and “computer processor” herein) 1705, which can be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735 that comprises a user interface (UI) 1740 for providing, for example, analysis of single-stranded nucleic acid molecule processing data. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, analyze single-stranded nucleic acid molecule processing data.

### EXAMPLES

### Example 1: Programmed Restriction Enzyme (PRE) Compositions

In this experiment, guide molecules direct strand cutting at specific programmed sites on a single-stranded nucleic acid sequence, as shown in FIGS. 1A-1J. The PRE compositions or methods provided herein can be referred to as DTECT, and the two terms can be used interchangeably herein. In FIG. 1A, a duplexed oligo (110) is formed from two individual oligos (115) which comprise a guide molecule PNA sequence (116) and a guide molecule nucleic acid sequence (117). The guide molecule PNA sequence (116) is located on the 3′ end of the oligo (115). The guide molecule PNA sequence (116) has a blocking moiety on its 3′ end. The guide molecule nucleic acid sequence (117) is located on the 5′ end of the oligo (115). The guide molecule nucleic acid sequence (117) is self-complementary on the non-target complement region (e.g., non-target binding region). The duplexed oligo (110) forms a complex with a restriction endonuclease (120) at selected sites on the guide molecule nucleic acid sequence (117) of each oligo (115). The duplexed oligo-restriction endonuclease complex binds to a target single strand nucleic acid sequence (100) at a target region (101).

FIG. 1B shows the cut site of a high frequency endonuclease (130) and the cut site of a low frequency endonuclease (135). If the high frequency endonuclease cuts, the duplexed oligo-restriction endonuclease complex will dissociate from the target. If the low frequency endonuclease cuts, it will lead to an open and extendable 3′ end on the target strand. FIG. 1C shows that the polymerase (140) extends off of the 3′ end, made available by the low frequency endonuclease. FIG. 1D shows that the polymerase (140) dissociates after completion of the synthesized strand (160), with the synthesized strand (160) having displaced one of the oligos (115) off of the duplexed oligo (110).

FIG. 1E again shows the cut site of a high frequency endonuclease (130) and the cut site of a low frequency endonuclease (135). At this stage, if the low frequency endonuclease cuts, the structure will be regenerated. However, if the high frequency endonuclease cuts, it will create an open and extendable 3′ end on the oligo strand. FIG. 1F shows that a polymerase (140) extends off of the 3′ end, made available by the high frequency endonuclease. The polymerase (140) displaced the guide molecule PNA sequence (116) and created a target synthesized strand (170).

FIG. 1G shows the cut site of a high frequency endonuclease (130). FIG. 1H shows that the high-frequency endonuclease cut and led to an open and extendable 3′ end on the target strand where the polymerase (140) bound and extended to create another target synthesized strand (171), displacing the previous target synthesized strand (170).

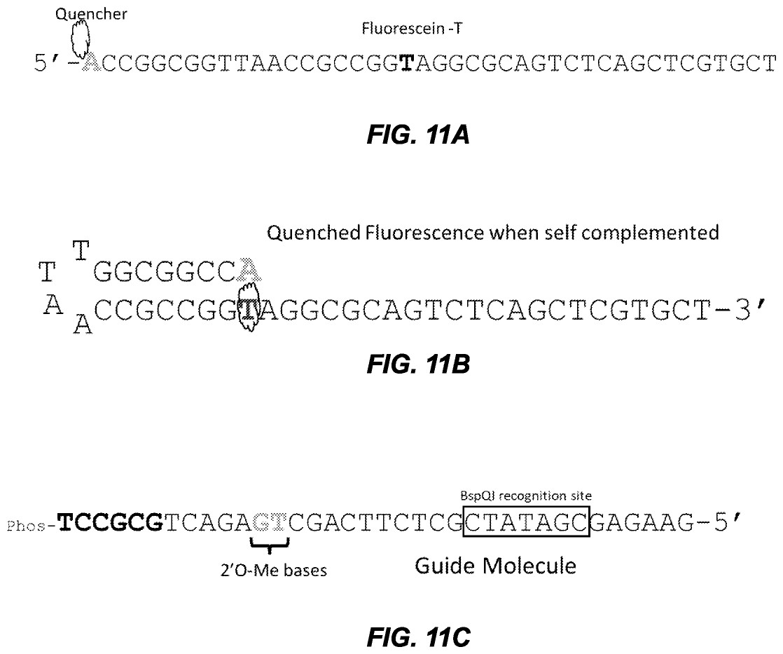

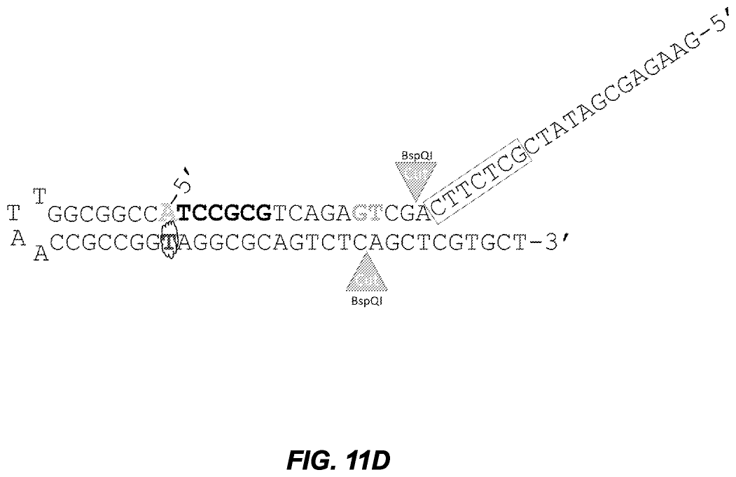

FIGS. 11 and 1J show the target synthesized strand (170), which was a complement to the target region (101) of the target single strand nucleic acid sequence (100), acted as a new target for the formation of additional synthesized strands (172) which represented copies of the target single strand nucleic acid sequence (100). The synthesized strands which are copies of the target single strand nucleic acid sequence (100) were the starting material for strand displacement amplification.

FIG. 1K depicts an exemplary completed extension on the new guide molecule.

In some experiments, the method continued, as seen in FIG. 1L, wherein endonucleolytic activity occurred on the second complementary strand oligo/extension product complex (170). FIG. 1M depicts a polymerase (140) extending of the 3′ end of the cut site of the second complementary strand of the oligo/extension product complex. Endolytic activity on the newly synthesized strand (42) occurred (FIG. 1N) and the displaced, single-stranded synthesized fragment (42) of FIG. 1O served as starting material for additional strand displacement amplification reactions.

### Example 2: Asymmetric Programmed Restriction Enzyme 1

In this experiment, guide molecules were designed with a single point mutation, such that they are still able to bind to the target DNA. Using the method of Example 1, additional strands were produced which do not contain the point mutation of the guide, but instead maintain products (other than the first synthesized strand) which have the correct complementary sequence of the target.

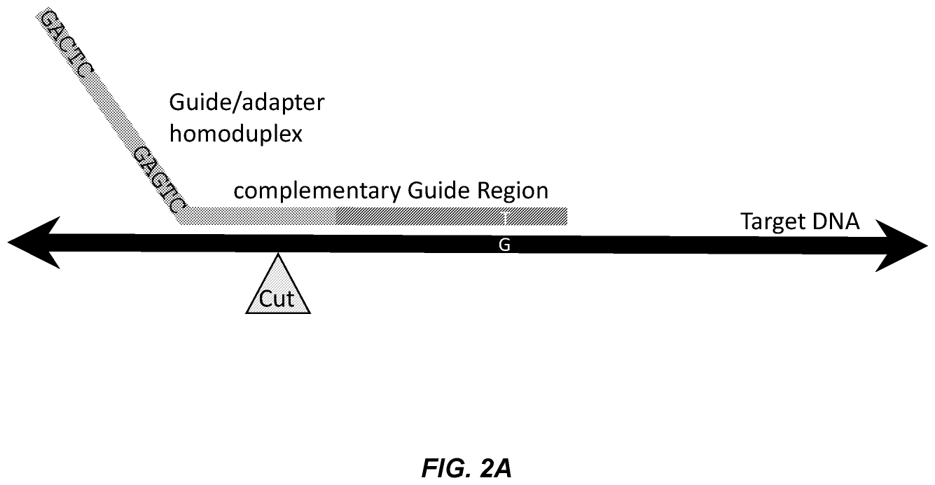

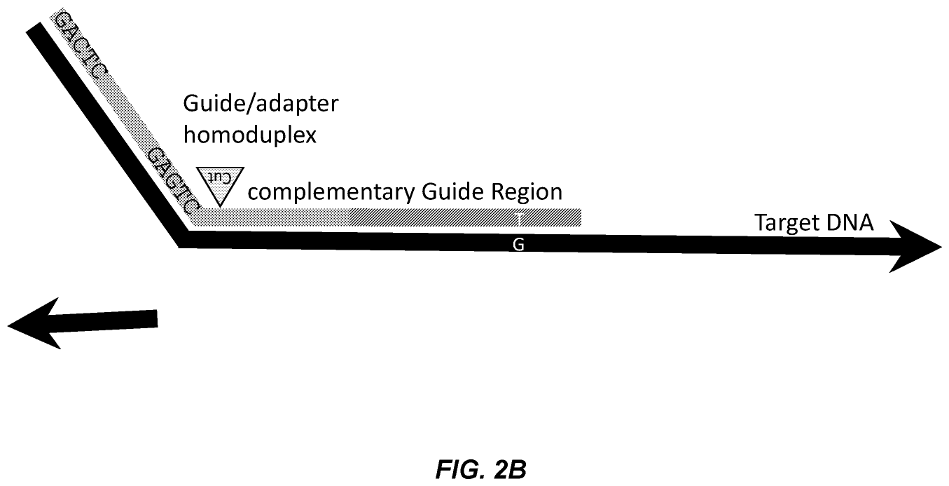

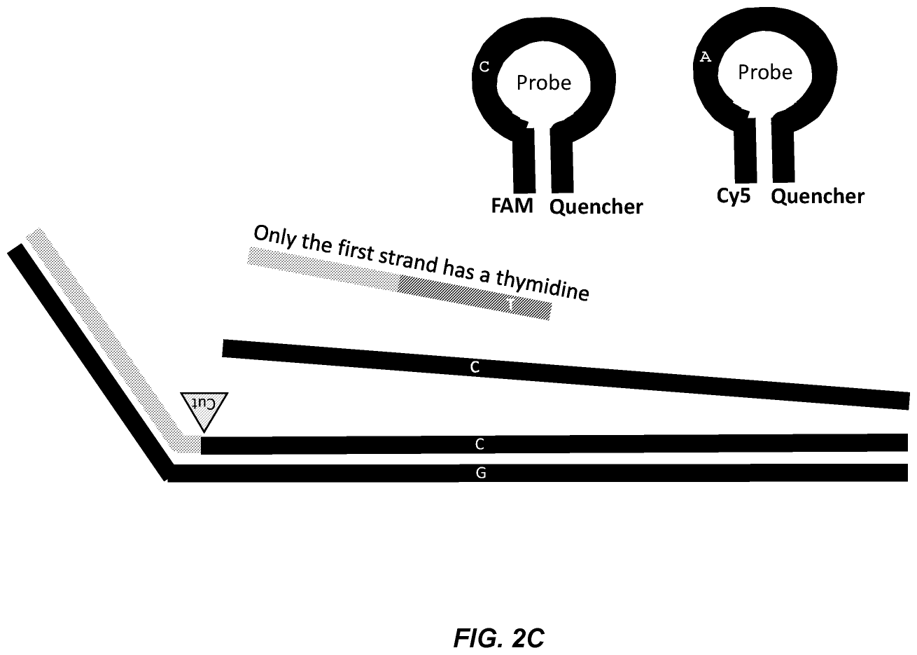

In FIG. 2A, the guide/adapter duplexed oligo with a single point mismatch (C to T) complexes with a target (template) single-stranded DNA. A low frequency endonuclease cuts the target DNA and digests inside the target region of the template region to create an extension of the template's new 3′ end (FIG. 2B). The high-frequency endonuclease site activity lead to cutting of the guide molecule, displacement, and synthesis of a new strand (FIG. 2C). This first strand had a thymidine, but all subsequent synthesized sequences had a cytosine instead, matching the complement of the original template region of the target strand.

In this experiment, the low frequency endonuclease activity was the critical step which allowed for the production of a product that fed into a strand displacement reaction.

Molecular beacon single nucleotide polymorphism (SNP) analysis was performed to differentiate between Primer extension (using a Cy5 fluorescent dye; black triangles) and guide oligo exonuclease activity (using a FAM fluorescent dye; grey circles).

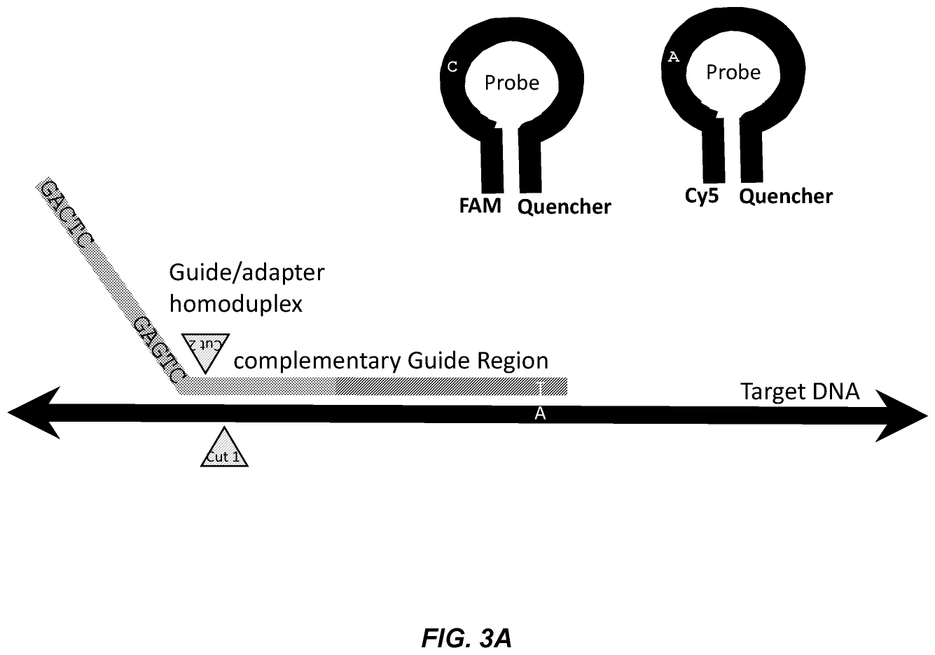

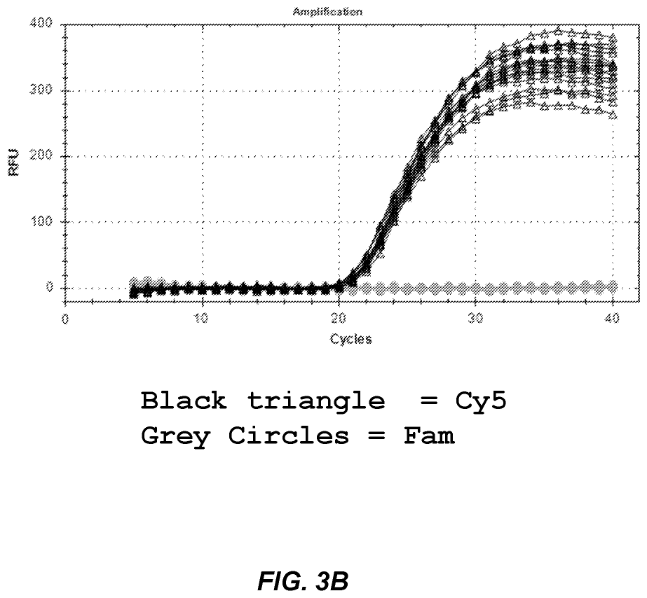

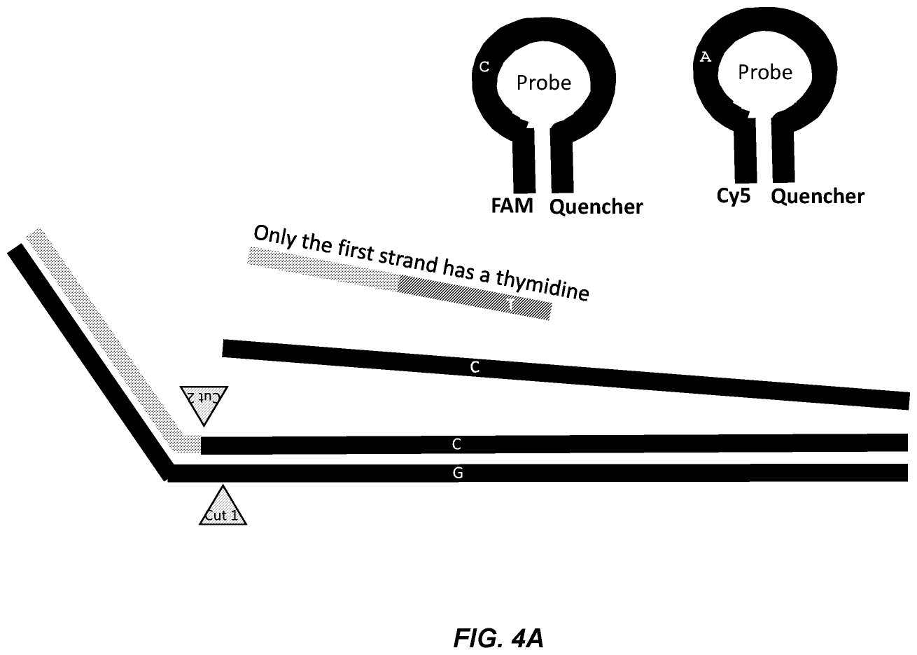

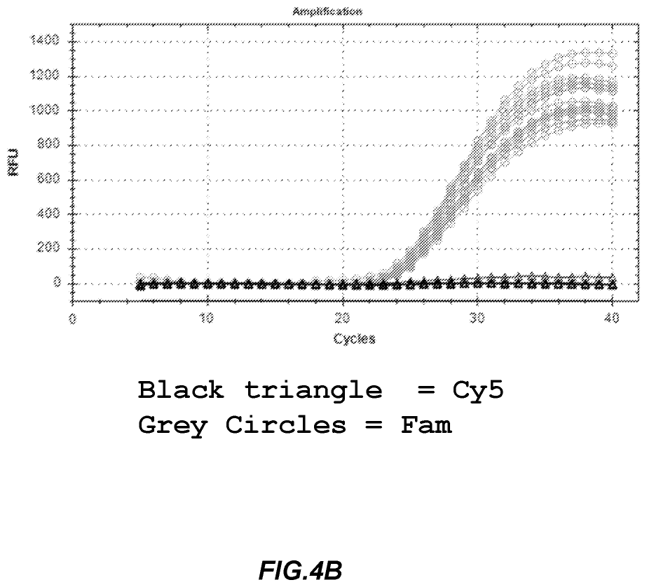

In the first control experiment, the guides and primers did not contain a mismatch. Results showed that amplification caused in increased fluorescence of the probe that contains the same sequence as the DNA target which binds to the amplified complementary target (FIG. 3B). However, when a mismatch was introduced to the guide sequence, the increase in fluorescence was to the probe that contained the same sequence as the DNA target as opposed to the probe that contains the compliment to the guide sequence (FIG. 4B). The sequences used in control experiment 1 and mismatch experiment 1 can be found in Table 1, FIG. 3A, and FIG. 4A.

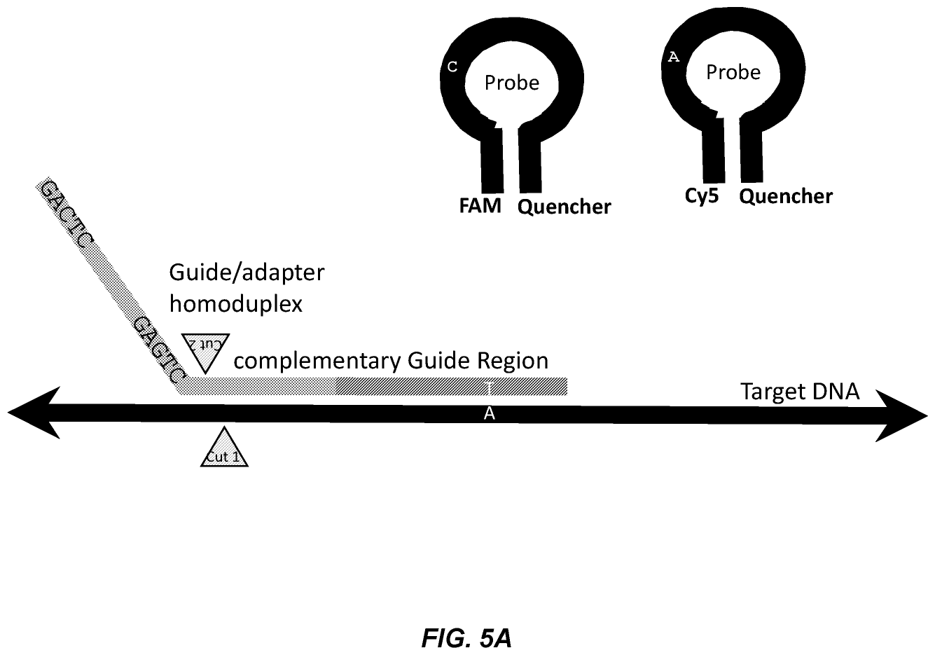

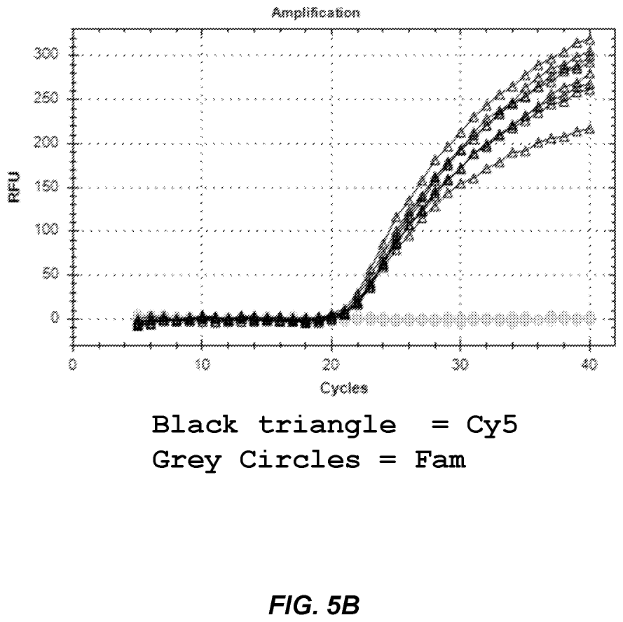

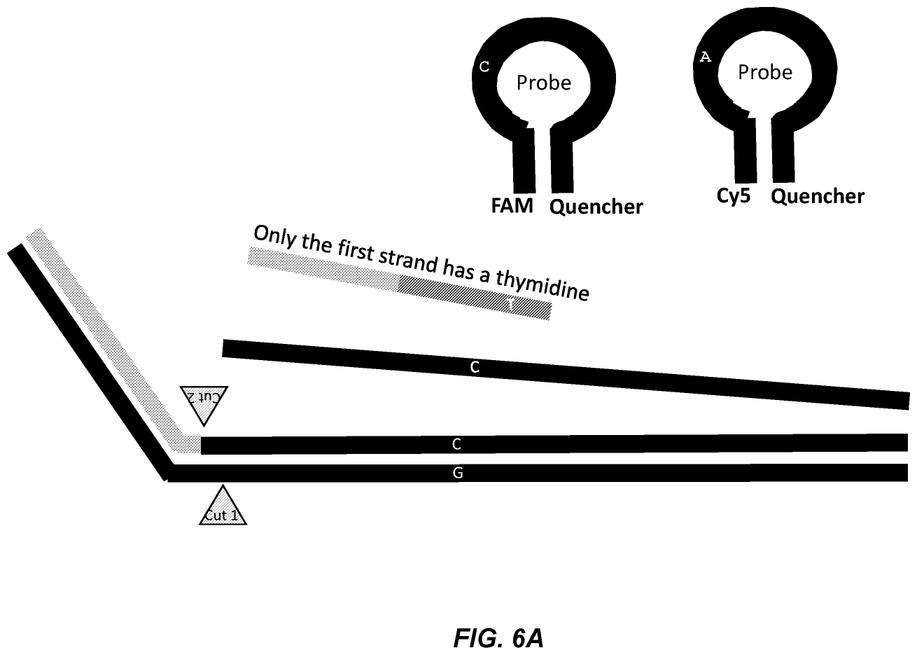

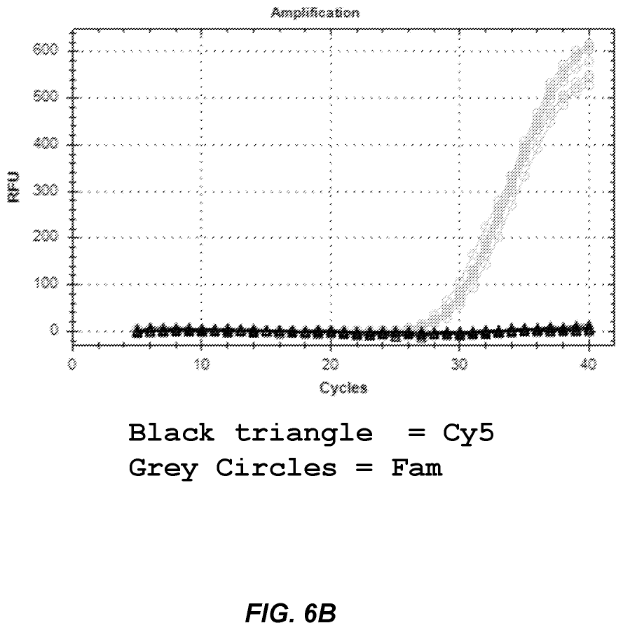

In the second control experiment, the guides and primers did not contain a mismatch. Results showed that amplification caused in increased fluorescence of the probe that contains the same sequence as the DNA target which binds to the amplified complementary target (FIG. 5B). However, when a mismatch was introduced to the guide sequence, the increase in fluorescence was to the probe that contained the same sequence as the DNA target as opposed to the probe that contains the compliment to the guide sequence (FIG. 6B). The sequences used in control experiment 2 and mismatch experiment 2 can be found in Table 1, FIG. 5A, and FIG. 6A. This example demonstrated that the oligonucleotides serve as guides of the endonucleolytic activity as opposed to the primers. Additionally, the example demonstrated that endonucleolytic activity occurs on both strands of the hybridized oligonucleotides in the complex.

TABLE 1
Sequences used in Example 2

| SEQ ID | | |
|---|---|---|
| NO | Description | Sequence |
| 1 | Control Experiment 1 | GACTCGGCCGAGTCGGTGCAATTTCAAGmUmGmUmUmUmU/3Phos/ |
| | Forward Guide Oligo | |
| 2 | Control Experiment 1 | GACTCGGCCGAGTCCACTTCAGCCTmCmAmAmCmUmU/3Phos/ |
| | Reverse Guide Oligo | |
| 3 | Control Experiment 1 | /56-FAM/CGCGCAACAAGTGTTTCAAATGATATCCTTGCGCG/ |
| | Probe Guide t/c 18-probe | 3BHQ_11 |
| 4 | Control Experiment 1 | /5Cy5/CCGCAAGTGTTTTAAATGATATCCTTGCGG/3BHQ_2/ |
| | Probe guide native probe | |
| 5 | Control Experiment 1 | GTTCTCTATGAGAACCAAAAATTGATTGCCAACCAATTTAATAGTG |
| | Target Sequence | CTATTGGCAAAATTCAAGAGTCACTTTCTTCCACAGCAAGTGCACTT |
| | | GGAAAACTTCAAGATGTGGTCAACCAAAATGCACAAGCTTTAAACA |
| | | CGCTTGTTAAACAACTTAGCTCCAATTTTGGTGCAATTTCAAGTGTT |
| | | TTAAATGATATCCTTTCACGTCTTGACAAAGTTGAGGCTGAAGTGC |
| | | AAATTGATAGGTTGATCACAGGCAGACTTCAAAGTTTGCAGACATA |
| | | TGTGACTCAACAATTAATTAGAGC |
| 6 | Mismatch Experiment 1 | GACTCGGCCGAGTCGGTGCAATTTCAAGmUmGmUmUmUmU/3Phos/ |
| | Forward Guide Oligo | |
| 7 | Mismatch Experiment 1 | GACTCGGCCGAGTCCACTTCAGCCTmCmAmAmCmUmU/3Phos/ |
| | Reverse Guide Oligo | |
| 8 | Mismatch Experiment 1 | /56- |
| | Probe Guide t/c 18-probe | FAM/CGCGCAACAAGTGTTTCAAATGATATCCTTGCGCG/3BHQ_1/ |
| 9 | Mismatch Experiment 1 | /5Cy5/CCGCAAGTGTTTTAAATGATATCCTTGCGG/3BHQ_2/ |
| | Probe guide native probe | |
| 10 | Mismatch Experiment 1 | GTTCTCTATGAGAACCAAAAATTGATTGCCAACCAATTTAATAGTG |
| | Target Sequence | CTATTGGCAAAATTCAAGAGTCACTTTCTTCCACAGCAAGTGCACTT |
| | | GGAAAACTTCAAGATGTGGTCAACCAAAATGCACAAGCTTTAAACA |
| | | CGCTTGTTAAACAACTTAGCTCCAATTTTGGTGCAATTTCAAGTGTT |
| | | TCAAATGATATCCTTTCACGTCTTGACAAAGTTGAGGCTGAAGTGC |
| | | AAATTGATAGGTTGATCACAGGCAGACTTCAAAGTTTGCAGACATA |
| | | TGTGACTCAACAATTAATTAGAGC |
| 11 | Control Experiment 2 | GACTCGGCCGAGTCGGTGCAATTTCAAGmUmGmUmUmUmU/3Phos/ |
| | Forward Guide Oligo | |
| 12 | Control Experiment 2 | GACTCGGCCGAGTCCACTTCAGCCTmCmAmAmCmUmU/3Phos/ |
| | Reverse Guide Oligo | |
| 13 | Control Experiment 2 | /56-FAM/CGCGCAACAAGTGTCTTAAATGATATCCTTGCGCGCGCGC |
| | Probe Guide t/c 18-probe | AACAAGTGTCTTAAATGATATCCTTGCGCG3BHQ_1/ |
| 14 | Control Experiment 2 | /5Cy5/CCGCAAGTGTTTTAAATGATATCCTTGCGG/3BHQ_2/ |
| | Probe guide native probe | |
| 15 | Control Experiment 2 | GTTCTCTATGAGAACCAAAAATTGATTGCCAACCAATTTAATAGTG |
| | Target Sequence | CTATTGGCAAAATTCAAGAGTCACTTTCTTCCACAGCAAGTGCACTT |
| | | GGAAAACTTCAAGATGTGGTCAACCAAAATGCACAAGCTTTAAACA |
| | | CGCTTGTTAAACAACTTAGCTCCAATTTTGGTGCAATTTCAAGTGTT |
| | | TTAAATGATATCCTTTCACGTCTTGACAAAGTTGAGGCTGAAGTGC |
| | | AAATTGATAGGTTGATCACAGGCAGACTTCAAAGTTTGCAGACATA |
| | | TGTGACTCAACAATTAATTAGAGC |
| 16 | Mismatch Experiment 2 | GACTCGGCCGAGTCGGTGCAATTTCAAGmUmGmUmUmUmU/3Phos/ |
| | Forward Guide Oligo | |
| 17 | Mismatch Experiment 2 | GACTCGGCCGAGTCCACTTCAGCCTmCmAmAmCmUmU/3Phos/ |
| | Reverse Guide Oligo | |
| 18 | Mismatch Experiment 2 | /56-FAM/CGCGCAACAAGTGTCTTAAATGATATCCTTGCGCGCGCGC |
| | Probe Guide t/c 18-probe | AACAAGTGTCTTAAATGATATCCTTGCGCG3BHQ_1/ |
| 19 | Mismatch Experiment 2 | /5Cy5/CCGCAAGTGTTTTAAATGATATCCTTGCGG/3BHQ_2/ |
| | Probe guide nat-probe | |
| 20 | Mismatch Experiment 2 | GTTCTCTATGAGAACCAAAAATTGATTGCCAACCAATTTAATAGTG |
| | Target Sequence | CTATTGGCAAAATTCAAGAGTCACTTTCTTCCACAGCAAGTGCACTT |
| | | GGAAAACTTCAAGATGTGGTCAACCAAAATGCACAAGCTTTAAACA |
| | | CGCTTGTTAAACAACTTAGCTCCAATTTTGGTGCAATTTCAAGTGTC |
| | | TTAAATGATATCCTTTCACGTCTTGACAAAGTTGAGGCTGAAGTGC |
| | | AAATTGATAGGTTGATCACAGGCAGACTTCAAAGTTTGCAGACATA |
| | | TGTGACTCAACAATTAATTAGAGC |

Abbreviations:
3Phos = 3′phosphate
BHQ = Black Hole Quencher ® dye (e.g., 2-[N-(2-hydroxyethyl)-4-[[2-methoxy-5-methyl-4-[(4-methyl-2-nitrophenyl)diazenyl]phenyl]diazenyl]anilino]ethanol phosphate ester)
m = 2′O-methyl RNA
Cy5 = Cyanine-5
FAM = fluorescein

### Example 3: Asymmetric Programmed Restriction Enzyme 2

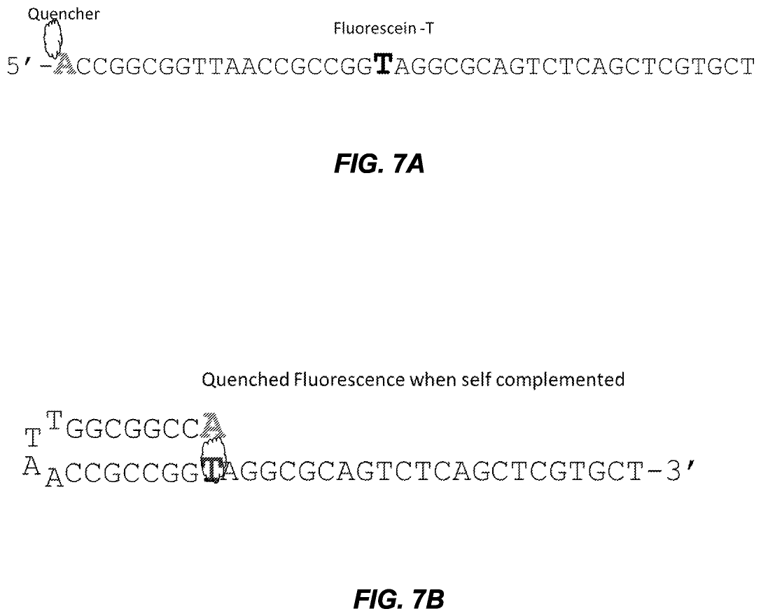

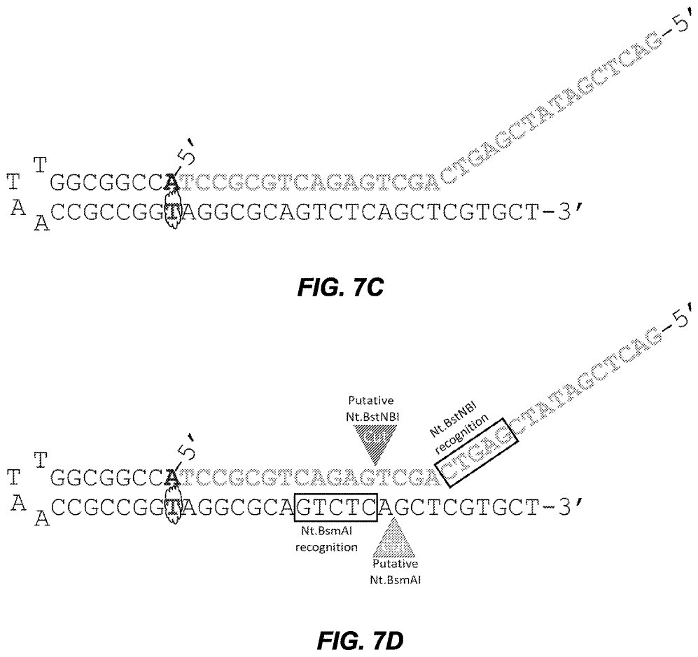

In this experiment, a detection molecule with an internal fluorophore-quencher pair, is used as the target. As seen in FIG. 7A-7B, the target molecule, when unpaired, self-complements and self-quenches. However, the molecule is fluorescent when double stranded. Using the method of Example 1, different terminal guides are tested for extension by a Bst polymerase. The different terminal guides, shown in Table 2, each contain a target non-complementary endonuclease recognition site.

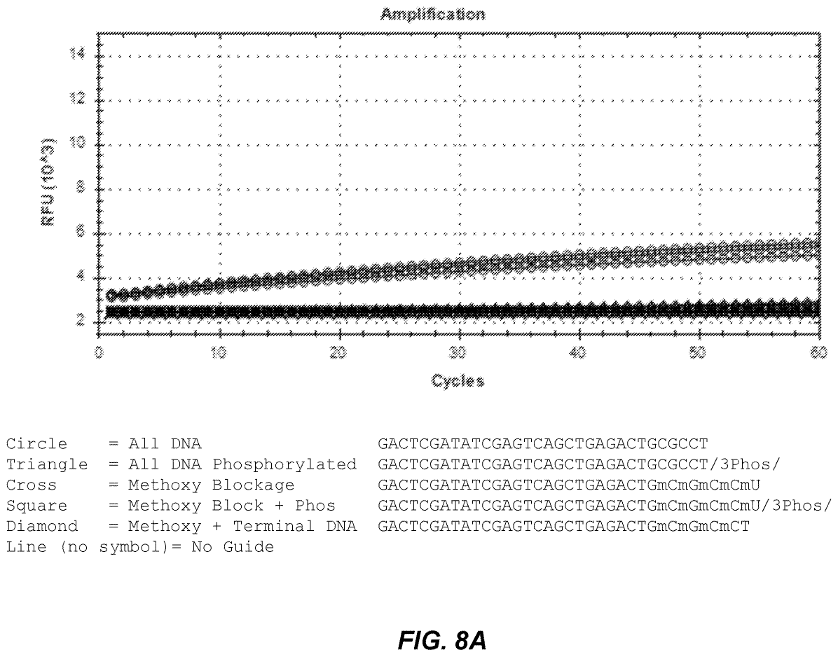

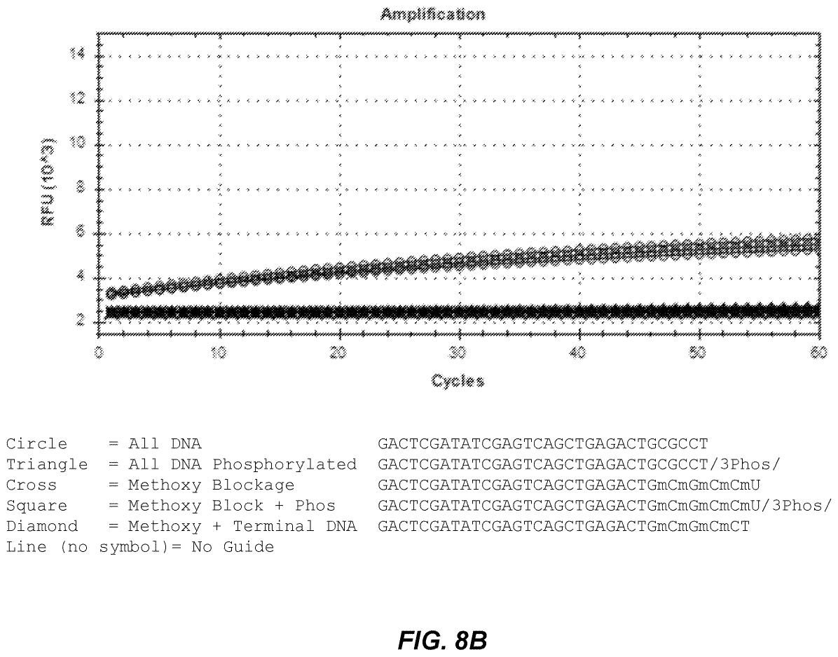

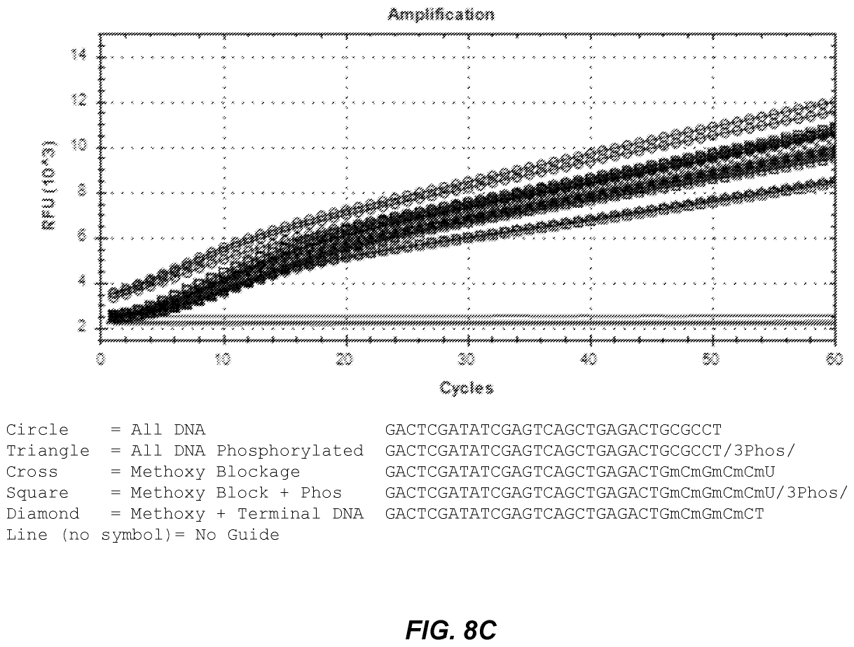

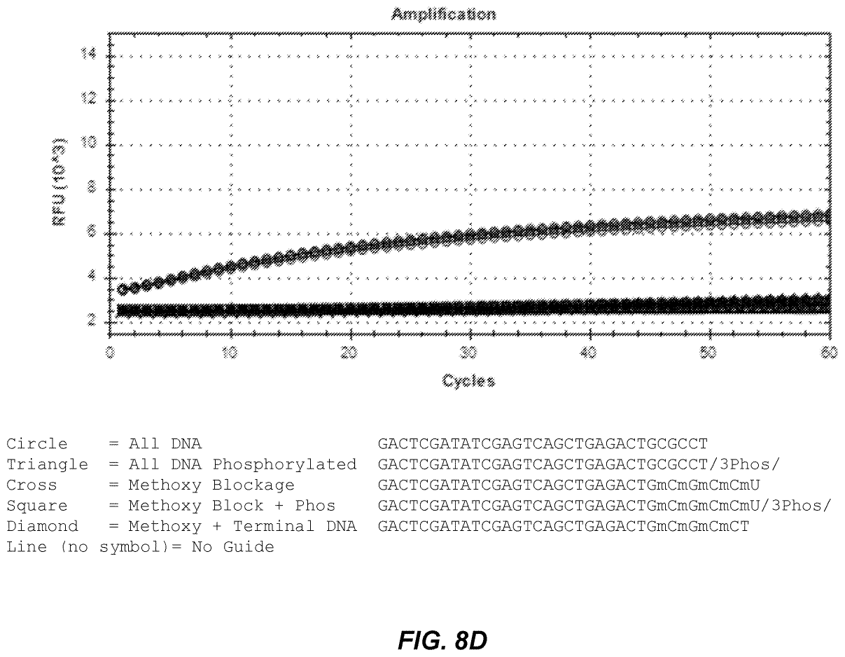

FIG. 7C-7D show the extension of the guide molecules and the endonuclease recognition sites. The N.BstNBI endonuclease had a temperature optimum at about 55° C., whereas the Nt.BsmAI endonuclease had a temperature optimum at about 37° C. Test conditions included Bst polymerase favored (FIG. 8A), Bst Polymerase with Nt.BsmAI temperatures favored (FIG. 8B), Bst polymerase with N.BstNBI temperatures favored (FIG. 8D), and Bst polymerase with N.BstNBI and Nt.BsmAI equally favored (FIG. 8C). In the reaction condition which favors Nt.BsmAI activity prior to N.BstNBI activity, leading to two stages of asymmetric enzyme activity, the thermocycler protocol held at 40° C. for 15 cycles (3.5 minutes) (preferred by Nt.BsmAI) followed by a temperature of 58° C. for 160 cycles (preferred by N.BstNBI).

In the reaction in which N.BstNBI made the primary cut, the oligo fell off at the reaction temperature due to an only 4 base overlap. In the reaction in which Nt.BsmAI made the primary cut, the Bst polymerase used the guide molecule as a target, activating N.BstNBI cutting activity. The resulting extension from the cut guide and opening of the prove increased Fam fluorescence. In the reaction in which N.BstNBI and Nt.BsmAI both cut, the oligo falls apart due to an only one base overlap.

Table 3 summarizes the results of FIGS. 8A-8D. Bst polymerase can extend off the 3′ end of DNA bases, but is blocked from extension via 2′-O-methyl RNA bases or phosphorylated bases. Nt.BsmAI did not have any effect on Bst extension. The system with both Nt.BsmAI and N.BstNBI showed that the two enzymes worked in conjunction to speed the reaction rate. This 2-enzyme system used temperature adjustments over time to maximize enzyme activity to asymmetrically cut the target, producing a defined/designed oligonucleotide that can be utilized in subsequent amplification reaction (e.g., SDA). In this two-enzyme system, the target cut by Nt.BsmAI is the rate limiting step. The N.BstNBI appeared to behave like a 2-enzyme asymmetric restriction enzyme system; this can be understood as the small subunit of N.BstNBI acting as the lower activity restriction endonuclease and the large subunit acting as the higher activity restriction endonuclease.

TABLE 2
Guide Molecules

| SEQ | | |
|---|---|---|
| ID NO | Description | Sequence |
| 21 | All DNA | GACTCGATATCGAGTC |
| | (Guide 1) | AGCTGAGACTGCGCCT |
| 22 | All DNA | GACTCGATATCGAGTC |
| | (phosphorylated) | AGCTGAGACTGCGCCT/ |
| | (Guide 2) | 3Phos |
| 23 | Methoxy Blockage | GACTCGATATCGAGTC |
| | (Guide 3) | AGCTGAGACTGmCmGm |
| | | CmCmU |
| 24 | Methoxy Blockage | GACTCGATATCGAGTC |
| | (phosphorylated) | AGCTGAGACTGmCmGm |
| | (Guide 4) | CmCmU/3Phos/ |
| 25 | Methoxy Blockage | GACTCGATATCGAGTC |
| | with a | AGCTGAGACTGmCmGm |
| | terminal DNA | CmCT |
| | (Guide 5) | |

TABLE 3
Amplification Summary

| | | | | Bst + |
|---|---|---|---|---|
| | Bst | Bst + | Bst + | N.BstNBI + |
| Guide | Polymerase | Nt.BsmAI | N.BstNBI | Nt.BsmAI |
| Guide 1 | Increased | Increased | Increased | Increased |
| | fluorescence | fluorescence | fluorescence | fluorescence |
| Guide 2 | No increased | No increased | Increased | Increased |
| | florescence | florescence | fluorescence | fluorescence |
| | (Extension | (Extension | | |
| | Blocked) | Blocked) | | |
| Guide 3 | No increased | No increased | Increased | Increased |
| | florescence | florescence | fluorescence | fluorescence |
| | (Extension | (Extension | | |
| | Blocked) | Blocked) | | |
| Guide 4 | No increased | No increased | Increased | Increased |
| | florescence | florescence | fluorescence | fluorescence |
| | (Extension | (Extension | | |
| | Blocked) | Blocked) | | |
| Guide 5 | Increased | Increased | Increased | Increased |
| | fluorescence | fluorescence | fluorescence | fluorescence |

### Example 4: Asymmetric Programmed Restriction Enzyme 3

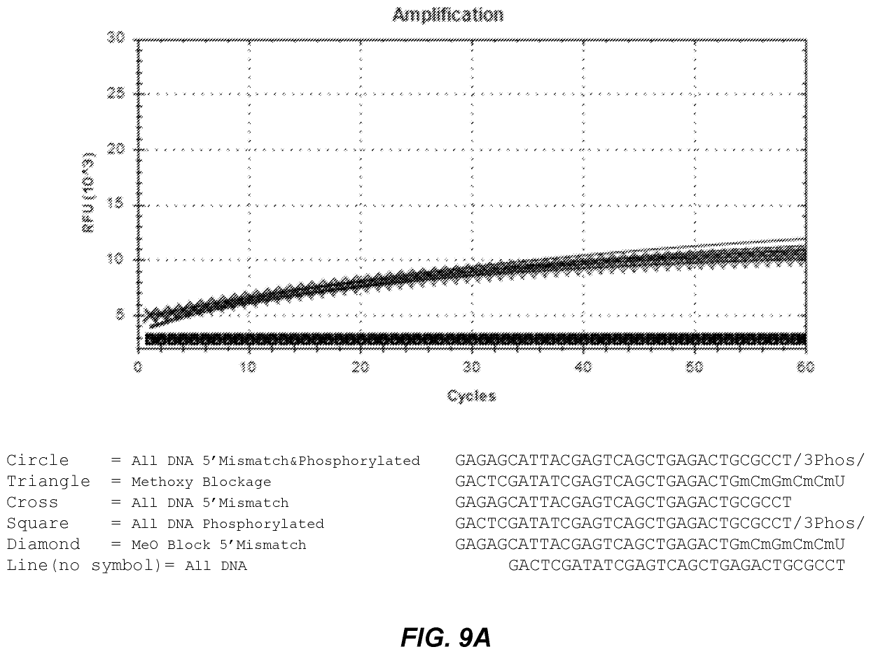

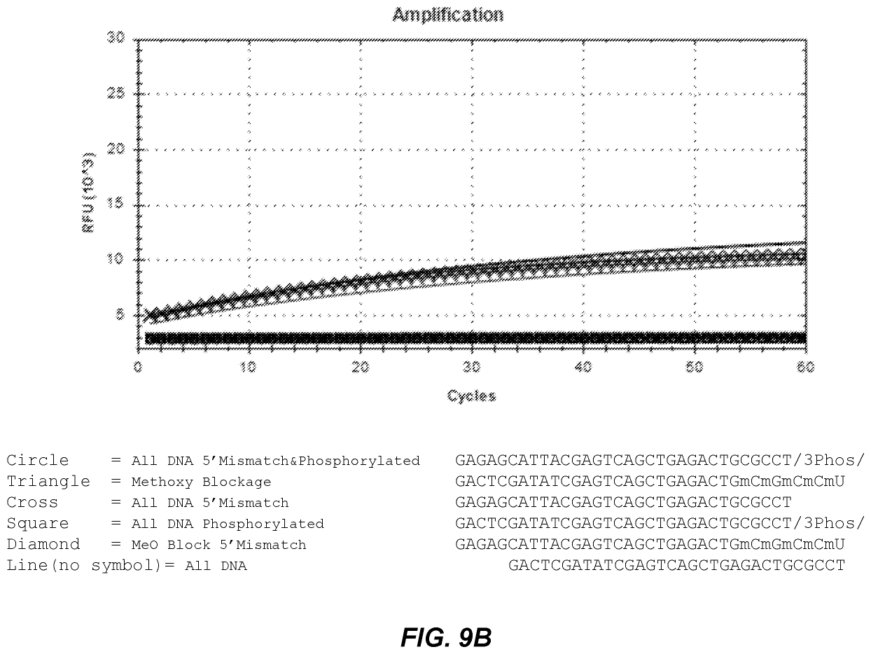

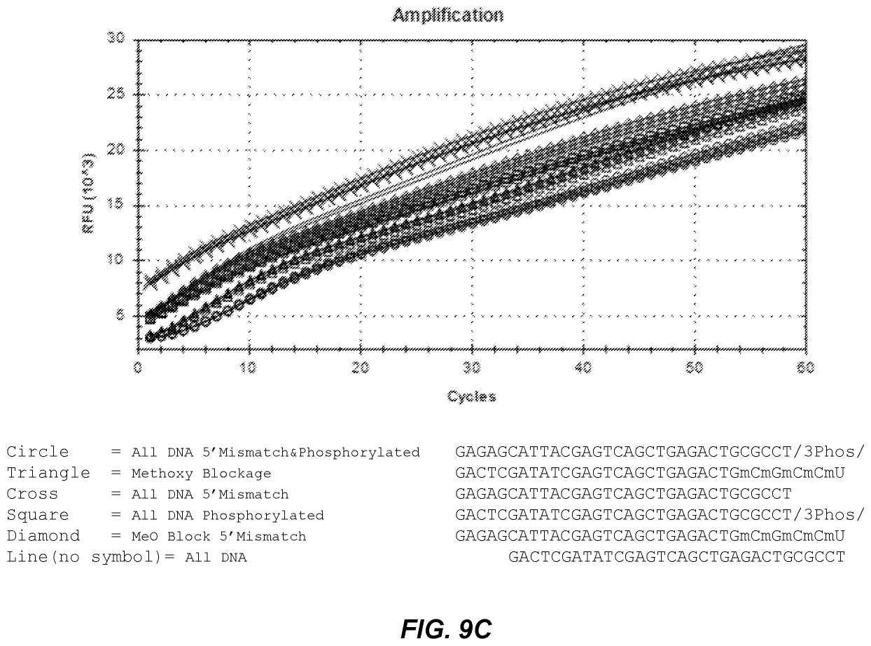

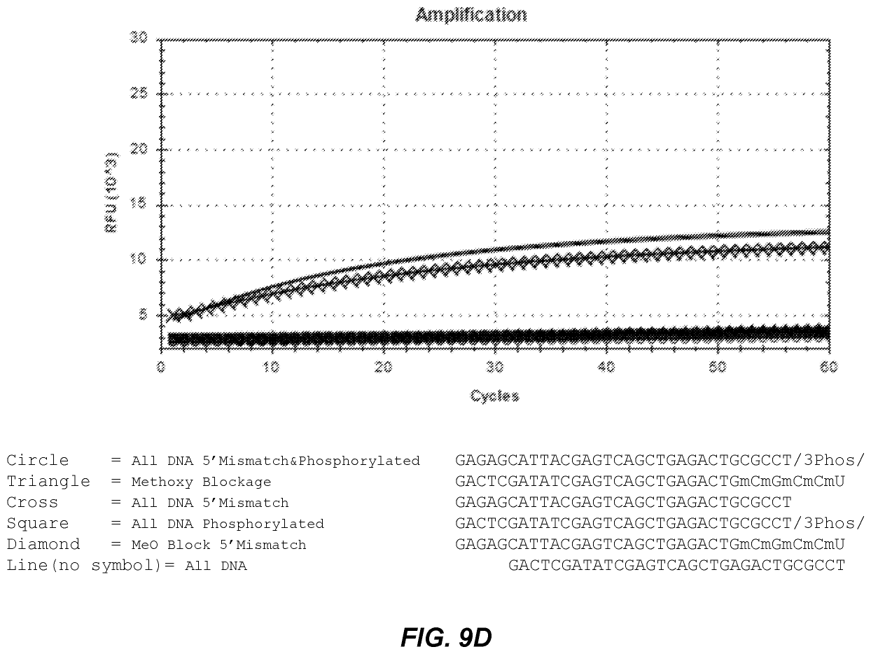

This experiment uses the methods of Example 3, using different guides. The different terminal guides are shown in Table 4. Test conditions included Bst polymerase favored (FIG. 9A), Bst Polymerase with Nt.BsmAI temperatures favored (FIG. 9B), Bst polymerase with N.BstNBI temperatures favored (FIG. 9D), and Bst polymerase with N.BstNBI and Nt.BsmAI equally favored (FIG. 9C).

Table 5 summarizes the results of FIGS. 9A-9D. Bst polymerase can extend off the 3′ end of DNA bases, but is blocked from extension off of 2′O methyl RNA bases or phosphorylated bases. Nt.BsmAI did not have any effect on Bst extension. In the system with both Nt.BsmAI and N.BstNBI, the two enzymes worked in conjunction to overcome the 3′ blocks. The N.BstNBI system showed slow release of the extension block.

TABLE 4
Guide Molecules

| SEQ ID | | |
|---|---|---|
| NO | Description | Sequence |
| 26 | All DNA 5′ Mismatch | GAGAGCATTACGAGTCAGC |
| | (phosphorylated) | TGAGACTGCGCCT/3Phos/ |
| | (Guide 6) | |
| 27 | Methoxy Blockage | GACTCGATATCGAGTCAGC |
| | (Guide 7) | TGAGACTGmCmGmCmCmU |
| 28 | All DNA 5′ Mismatch | GAGAGCATTACGAGTCAGC |
| | (Guide 8) | TGAGACTGCGCCT |
| 29 | All DNA | GACTCGATATCGAGTCAGC |
| | (phosphorylated) | TGAGACTGCGCCT/3Phos/ |
| | (Guide 9) | |
| 30 | Methoxy Blockage | GAGAGCATTACGAGTCAGCT |
| | with 5′ Mismatch | GAGACTGmCmGmCmCmU |
| | (Guide 10) | |
| 31 | All DNA | GACTCGATATCGAGTCAGCT |
| | (Guide 11) | GAGACTGCGCCT |

TABLE 5
Amplification Summary

| | | | | Bst + |
|---|---|---|---|---|
| | Bst | Bst + | Bst + | N.BstNBI + |
| Guide | Polymerase | Nt.BsmAI | N.BstNBI | Nt.BsmAI |
| Guide 6 | No increased | No increased | Increased | Increased |
| | florescence | florescence | fluorescence | fluorescence |
| | (Extension | (Extension | | (slow) |
| | Blocked) | Blocked) | | |
| Guide 7 | No increased | No increased | Increased | Increased |
| | florescence | florescence | fluorescence | fluorescence |
| | (Extension | (Extension | | (slow) |
| | Blocked) | Blocked) | | |
| Guide 8 | Increased | Increased | Increased | Increased |
| | fluorescence | fluorescence | fluorescence | fluorescence |
| Guide 9 | No increased | No increased | Increased | Increased |
| | florescence | florescence | fluorescence | fluorescence |
| | (Extension | (Extension | | (slow) |
| | Blocked) | Blocked) | | |
| Guide 10 | No increased | No increased | Increased | Increased |
| | florescence | florescence | fluorescence | fluorescence |
| | (Extension | (Extension | | (slow) |
| | Blocked) | Blocked) | | |
| Guide 11 | Increased | Increased | Increased | Increased |
| | fluorescence | fluorescence | fluorescence | fluorescence |

### Example 5: LAMP Enhancement with PRE Priming

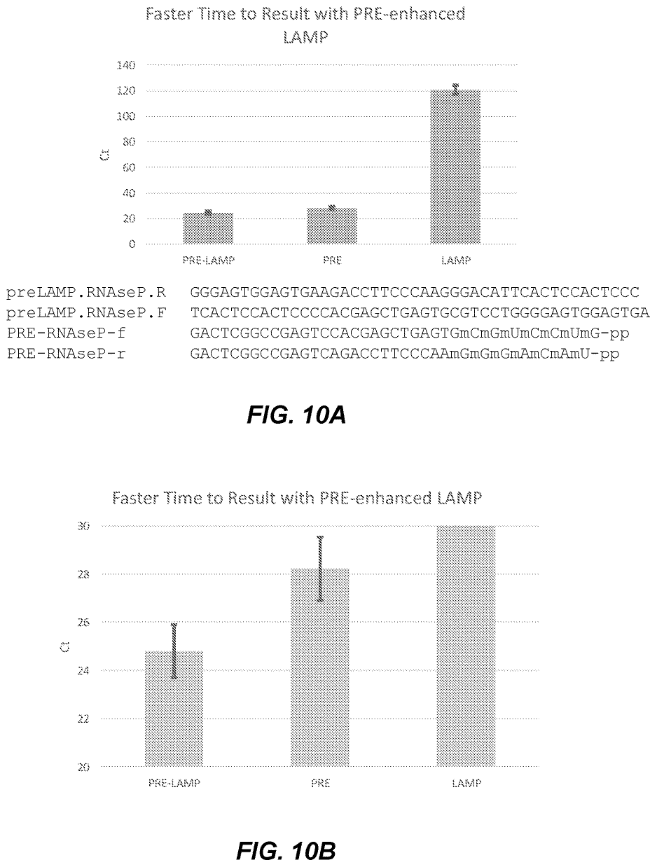

This experiment compared Loop-Mediated Isothermal Amplification (LAMP), PRE (also known as DTECT), and LAMP combined with PRE priming. PRE was performed as described in Example 1. Results show that PRE-enhanced LAMP had a lower cycle threshold than either PRE alone or LAMP alone (FIGS. 10A-10B). Amplification enhancement with PRE used approximately 100 copies hRNA per reaction. Table 6 shows guides used in this experiment.

This experiment showed that symmetric endonuclease activity can create the starting product for isothermal amplification systems such as LAMP and decrease time to results. The reaction rate increase is not limited to SDA.

TABLE 6
LAMP Guide Molecules

| SEQ | | |
|---|---|---|
| ID NO | Description | Sequence |
| 32 | preLAMP RNAse | GGGAGTGGAGTGAAGACCTT |
| | reverse | CCCAAGGGACATTCACTCCA |
| | | CTCCC |
| 33 | preLAMP RNAse | TCACTCCACTCCCCACGAGC |
| | forward | TGAGTGCGTCCTGGGGAGTG |
| | | GAGTGA |
| 34 | PRE RNAse forward | GACTCGGCCGAGTCCACGAG |
| | | CTGAGTGmCmGmUmCmCmUm |
| | | G/3Phos/ |
| 35 | PRE RNAse reverse | GACTCGGCCGAGTCAGACCT |
| | | TCCCAAmGmGmGmAmCmAmU |
| | | /3Phos/ |

### Example 6: Enzyme Activity Modification Through Guide Design

This experiment uses the same detection molecules with internal fluorophore-quencher pairs as Example 3 (FIGS. 11A-11B). However, this experiment also includes further modified guides which modify enzyme activity (FIG. 11C).

This experiment uses the enzyme BspQI, which has a primary cut site next to its recognition site (boxed) and a forced cut site on the guide. Nt.BspQI, also used in this experiment as a control, is a triple mutant form of BspQI which has top-strand DNA nicking activity.

In the reaction in which BspQI cut at its primary cut site, the oligo fell off at the reaction temperature due to only 1 base overlap. In the reaction in which BspQI was forced to make the asymmetric secondary cut, the Bst polymerase used the guide molecule as a target, activating BspQI primary cutting activity. The resulting extension from the cut guide and opening of the prove increased Fam fluorescence. In the reaction in which BspQI cut at both cut sites, the oligo falls apart due to an only three base overlap. The guides used in this experiment are found in Table 7.

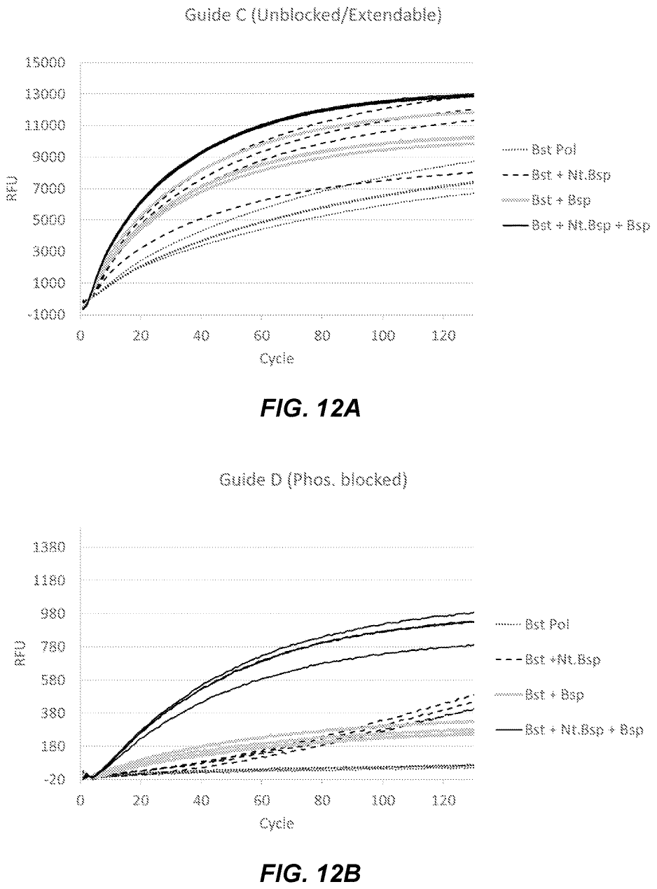

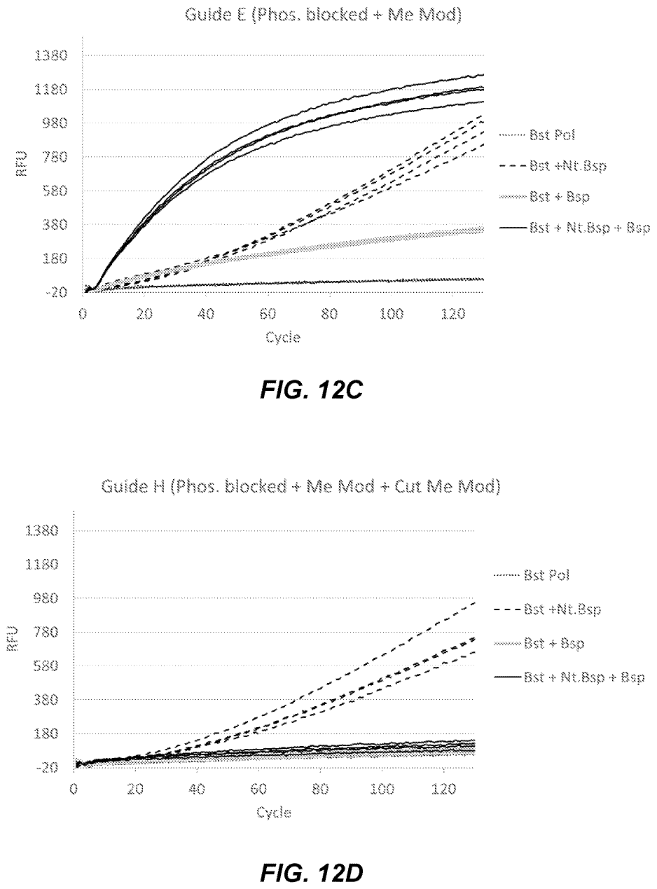

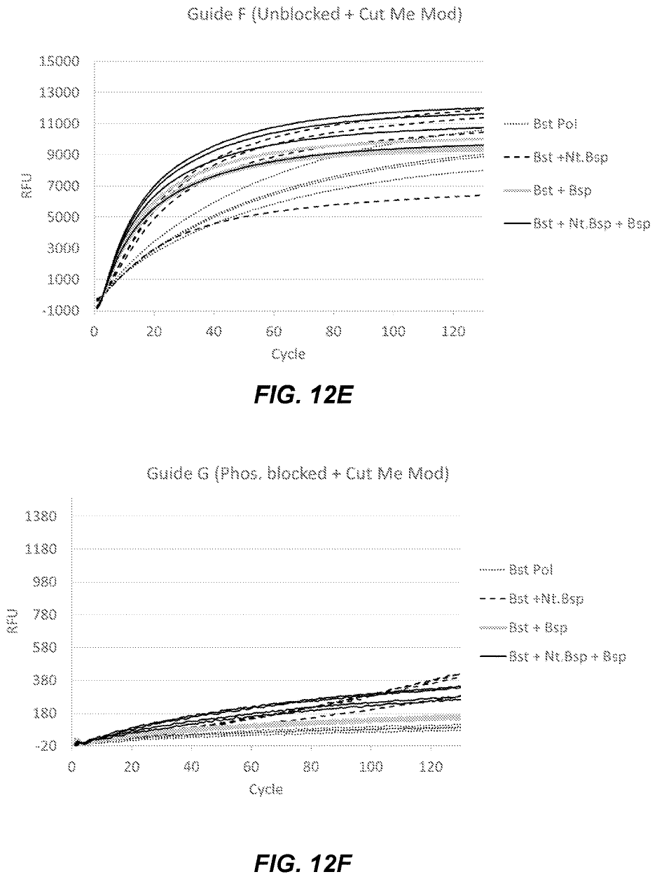

Guide C showed increased fluorescence with endonuclease from additional copies made which implies that either Bst polymerase activity is faster than endonuclease activity or the endonuclease keeps the targets together after cutting to allow Bst to extend (FIG. 12A). Guide D showed increased fluorescence with endonuclease and Bst extension was blocked without endonuclease which implies that asymmetric endonuclease activity allows the bypass of guide blockage and that Nt.BspQI has bottom strand nuclease activity (FIG. 12B). Guide E showed increased fluorescence with endonuclease; that Bst extension was blocked without endonuclease; and increased fluorescence with Nt.BspQI (FIG. 12C). This implies that asymmetric endonuclease activity allows the bypass of guide blockage; that Nt.BspQI has bottom strand nuclease activity; and that enzymatic activity is tunable with different guide chemistries. Guide H showed increased fluorescence with nickase; that Bst extension was blocked without endonuclease; and that a 2′O-MeO on the opposite bottom endonuclease cut prevents fluorescence reporting (FIG. 12D). This implies that the inhibition of the BspQI bottom cut allowed the guide to be cut only on the top, ‘shorting’ the system. Additionally, the top strand cutting of Nt.BspQI may not be as efficient under these conditions. Guide F showed increased fluorescence with endonuclease from additional copies made which implies that either Bst polymerase activity is faster than endonuclease activity or the endonuclease keeps the targets together after cutting to allow Bst to extend (FIG. 12E). Guide G showed minimal increased fluorescence, implying either inhibition of both endonucleases or enhancement of nuclease activity over polymerase activity (FIG. 12F).

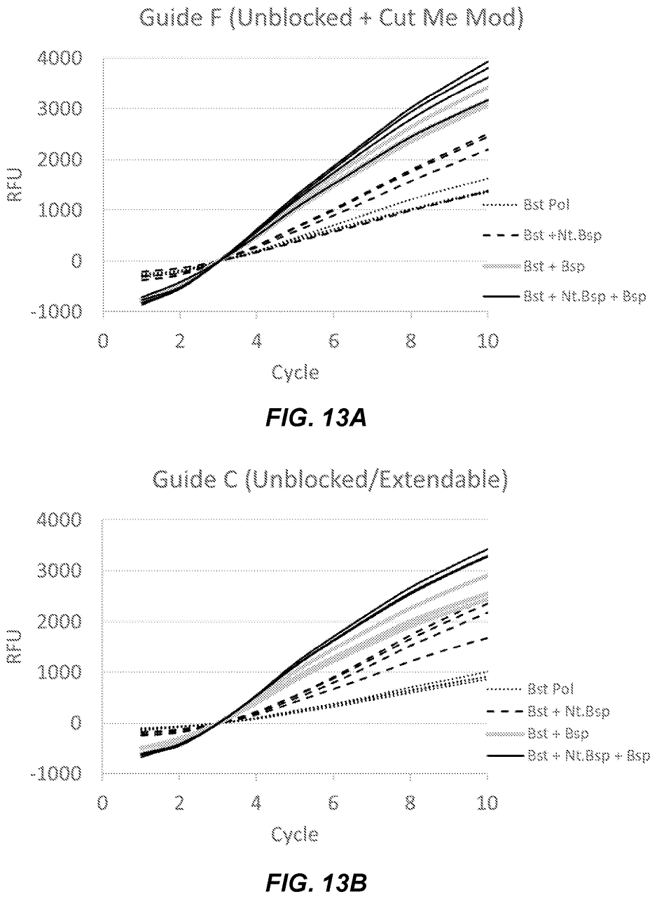

FIGS. 13A-13B compare Guide F to Guide C, showing that there was a slight enhancement of signal when methoxylation on the guide was in proximity to the cut site.

This experiment showed that guide molecule extension can be blocked by various moieties and that restriction enzyme activity can be modified to behave asymmetrically to accelerate one side cutting activity over another. Modifications of guide molecules can allow the endonuclease activity occurs on the target in a desired and specific location while relief of the blocking of guide molecules can be achieved by modifying the activity of endonuclease(s) in the system or modifying the activity of the strand displacement polymerase in the system.

TABLE 7
Guide Molecules

| SEQ ID | | |
|---|---|---|
| NO | Description | Sequence |
| 36 | Guide C | GAAGAGCGATATCGCTCTTC |
| | | AGCTGAGACTGCGCCT |
| 37 | Guide D | GAAGAGCGATATCGCTCTTC |
| | | AGCTGAGACTGCGCCT/ |
| | | 3Phos/ |
| 38 | Guide E | GAAGAGCGATATCGCTCTTC |
| | | AGCTGAGACTmGmCmGmCmC |
| | | mU/3Phos/ |
| 39 | Guide F | GAAGAGCGATATCGCTCTTC |
| | | AGCmUmGAGACTGCGCCT |
| 40 | Guide G | GAAGAGCGATATCGCTCTTC |
| | | AGCmUmGAGACTGCGCCT/3 |
| | | Phos/ |
| 41 | Guide H | GAAGAGCGATATCGCTCTTC |
| | | AGCmUmGAGACTmGmCmGmC |
| | | mCmU/3Phos/ |

### Example 7: Isothermal SDA after the Production of a Restriction Digestion Target Product

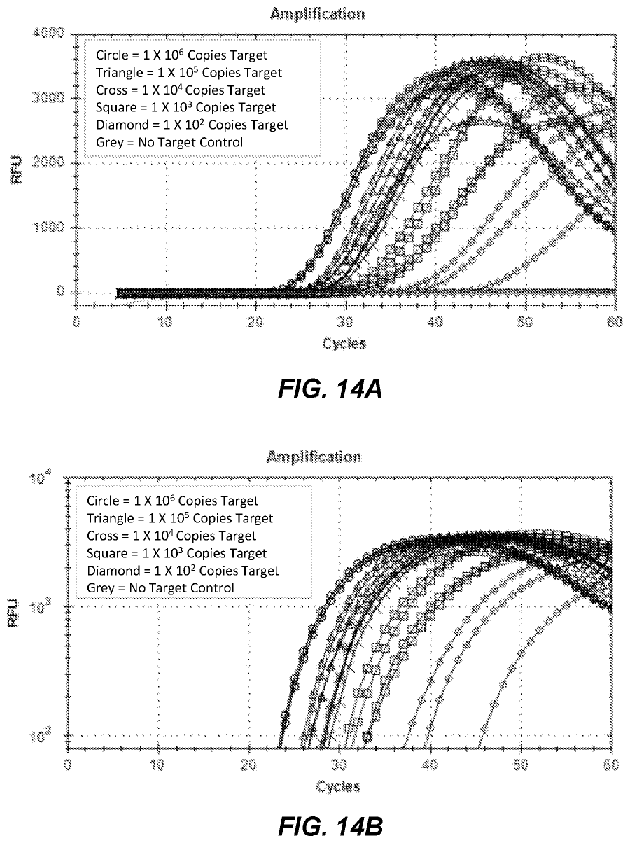

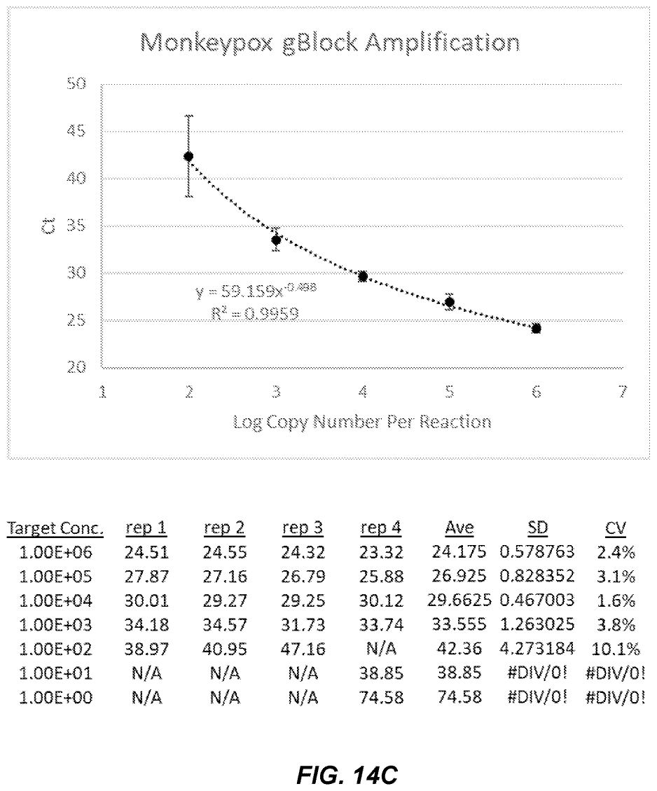

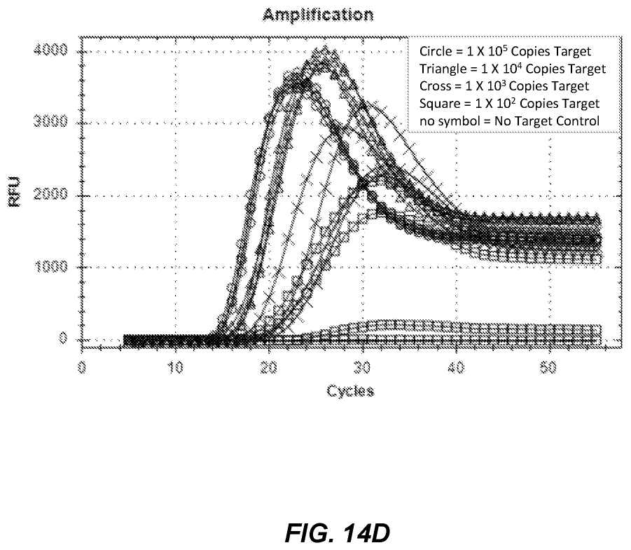

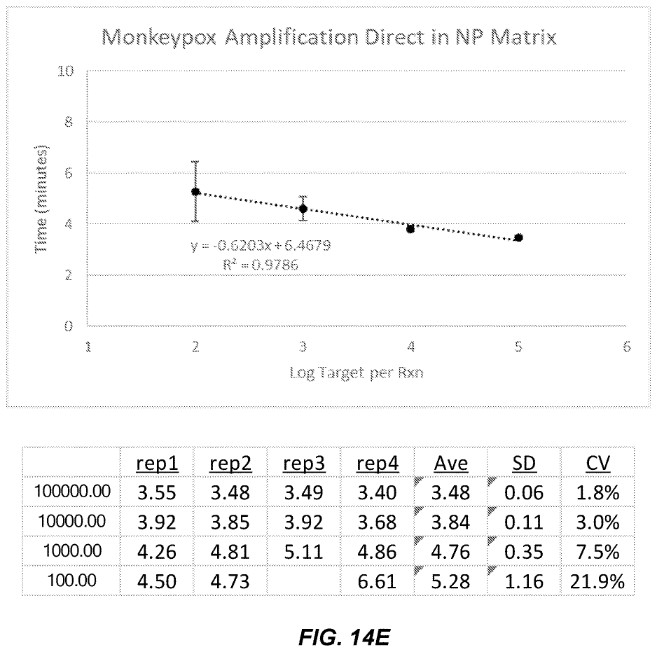

This experiment used the method of Example 1 and then ran isothermal SDA to see the effect on the resulting amplification of target products. A monkeypox titration 10-fold dilution series was performed (FIGS. 14A-14B). Results of the amplification are shown in FIG. 14C. Monkeypox virus amplification was also done in an NP matrix direct amplification reaction (FIGS. 14D-14E).

### Example 8: Multiplexed Isothermal Amplification

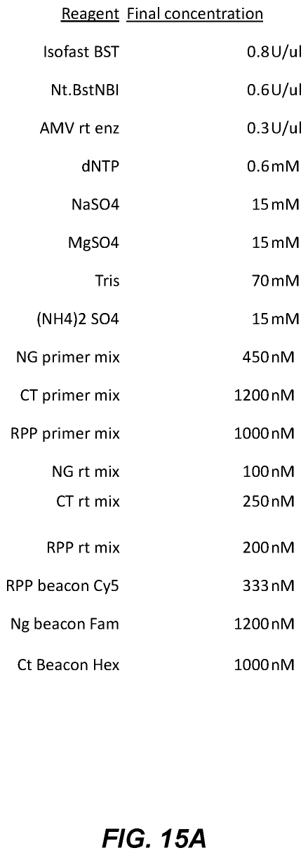

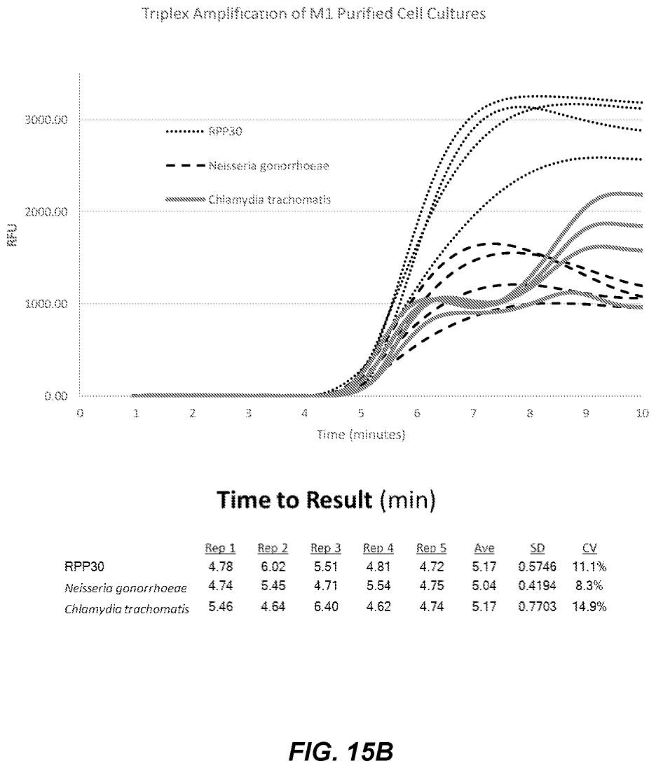

This experiment used the method of Example 1 and ran isothermal SDA on samples of Neisseria gonorrhoeae, Chlamydia trachomatis, and RPP30. Reaction conditions of the triplex experiment are shown in FIG. 15A. M1 Sample Prep® was performed using 400 μL of frozen sample culture, resuspended in approximately 800 μL of total culture. The target quantitation was based on an M1 Sample Prep® with an assumed 100% recovery from sample culture. Performance of the triplex isothermal reaction showed amplification for all three reactions (FIG. 15B).

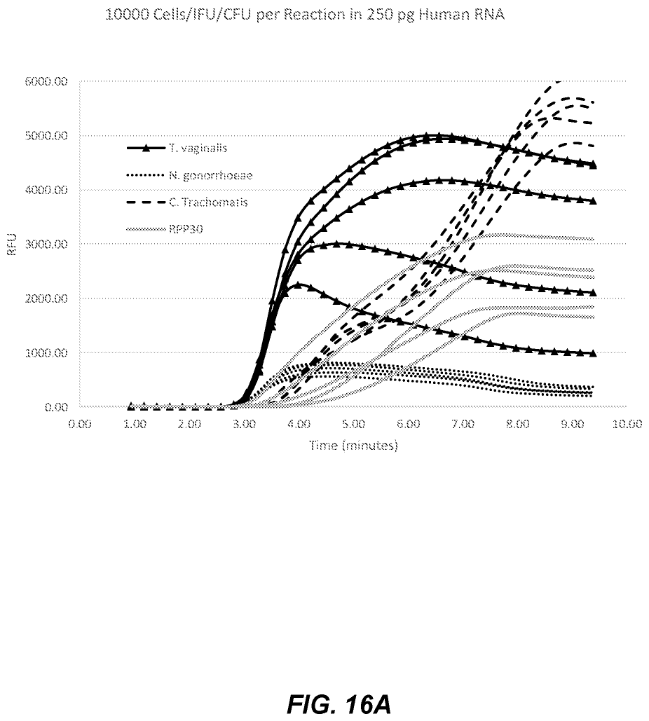

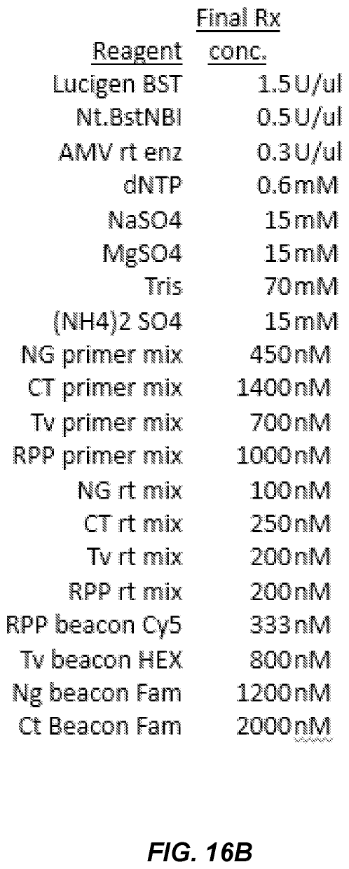

This experiment also used the method of Example 1 and ran isothermal SDA on samples of Neisseria gonorrhoeae, Chlamydia trachomatis, RPP30, and Trichomonas vaginalis. As in the triplex experiment, the tetraplex isothermal reaction showed competing amplification for the four reaction conditions (FIG. 16A). Reaction conditions of the tetraplex experiment are shown in FIG. 16B.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of processing a single-stranded nucleic acid molecule comprising a target sequence, said method comprising:
(a) contacting said single-stranded nucleic acid molecule with a guide complex comprising a guide polynucleotide under conditions where said guide polynucleotide hybridizes to said single-stranded nucleic acid molecule, wherein said guide polynucleotide comprises:
(i) a non-target binding region comprising a restriction endonuclease recognition sequence for an enzyme that is a type IIs restriction enzyme,
(ii) a target binding region configured to hybridize to said target sequence, and
(iii) a blocked 3′ end non-extendable by a polymerase; and
(b) introducing said type IIs restriction enzyme under conditions sufficient to cause said type IIs restriction enzyme to bind said restriction endonuclease recognition sequence and cut within said target sequence.

2. The method of claim 1, wherein in (b) said cut exposes an extendable 3′ end of said target sequence.

3. The method of claim 2, further comprising extending said extendable 3′ end using a polymerase.

4. A method of amplifying a single-stranded nucleic acid molecule comprising a target sequence, said method comprising:
(a) contacting said single-stranded nucleic acid molecule with a guide complex comprising a guide polynucleotide under conditions where said guide polynucleotide hybridizes to said single-stranded nucleic acid molecule, wherein said guide polynucleotide comprises:
(i) a non-target binding region comprising a restriction endonuclease recognition sequence for a type IIs restriction enzyme,
(ii) a target binding region configured to hybridize to said target sequence, and
(iii) a blocked 3′ end non-extendable by a polymerase;
(b) introducing said type IIs restriction enzyme under conditions sufficient to cause said type IIs restriction enzyme to bind said restriction endonuclease recognition sequence and cut within said target sequence to generate an extendable 3′ end; and
(c) extending said extendable 3′ end of said target sequence using a polymerase.

5. The method of any one of claims 1-4, wherein said guide polynucleotide is a first guide polynucleotide, and said guide complex comprises a second guide polynucleotide, wherein said second guide polynucleotide comprises (i) a non-target binding region that is complementary with said non-target binding region of said first guide polynucleotide and (ii) a target binding region configured to hybridize to said target sequence.

6. The method of claim 5, wherein when said first guide polynucleotide of said guide complex is hybridized to said target polynucleotide sequence, said target binding region of said second guide polynucleotide is not hybridized to said target sequence.

7. The method of claim 5 or 6, wherein said first guide polynucleotide and said second guide polynucleotide hybridize to form a dimer.

8. The method of claim 7, wherein said first guide polynucleotide and said second guide polynucleotide hybridize via said non-target binding region of said first guide polynucleotide and said second guide polynucleotide to form said dimer having a double-stranded binding region.

9. The method of claim 8, wherein said double-stranded binding region comprises said restriction endonuclease recognition sequence.

10. The method of claim 8, wherein said type IIs restriction enzyme binds to said double-stranded binding region of said dimer.

11. A method of amplifying a single-stranded nucleic acid molecule comprising a target sequence, said method comprising:
(a) contacting a guide complex with said single-stranded nucleic acid molecule, wherein said guide complex comprises:
(i) a first guide polynucleotide comprising, from 5′ to 3′, a non-target binding region and a target binding region that hybridizes with said target sequence of said single-stranded nucleic acid molecule, and
(ii) a second guide polynucleotide that hybridizes with said non-target binding region of said first guide molecule to form a double-stranded binding region, wherein said double-stranded binding region binds to an enzyme;
(b) cutting said target sequence using said enzyme to expose an extendable 3′ end of said target sequence;
(c) extending said extendable 3′ end of said target sequence with a polymerase to generate an extension product, wherein said extension product displaces said second guide polynucleotide;
(d) cutting said first guide polynucleotide within said target binding region to expose an extendable 3′ end of said first guide polynucleotide; and
(e) extending said extendable 3′ end of said first guide polynucleotide using said polymerase to generate a complementary molecule of said target sequence of said single-stranded nucleic acid molecule, thereby amplifying said single-stranded nucleic acid molecule.

12. The method of claim 11, wherein said second guide polynucleotide comprises, from 5′ to 3′ (i) a non-target binding region that hybridizes with said non-target binding region of said first guide polynucleotide and (ii) a target binding region configured to hybridize with said target sequence.

13. The method of claim 11 or 12, further comprising, prior to (b), cutting said first guide polynucleotide within said target binding region using said enzyme, wherein said guide complex dissociates from said single-stranded nucleic acid molecule.

14. The method of any one of claims 11-13, further comprising repeating (d) and (e) to generate a plurality of complementary molecules of said target sequence of said single-stranded nucleic acid molecule.

15. The method of any one of claims 11-14, wherein an additional guide complex binds to said complementary molecule.

16. The method of claim 15, further comprising using said complementary molecule with said additional guide complex bound thereto as a starting template to generate copies of said target molecule.

17. The method of any one of claims 11-16, wherein said enzyme is a type IIs restriction enzyme.

18. The method of any one of claims 1-10 and 17, wherein said type IIs restriction enzyme comprises N.BstNBI, N.Bst9 I, N.BspD6I, a functional fragment thereof, or a combination thereof.

19. The method of any one of claims 11-18, wherein said guide polynucleotide comprises a blocked 3′ end non-extendable by a polymerase.

20. The method of any one of claims 1-10 and 19, wherein said blocked 3′ end comprises a PNA, a modified base, a phosphate group, a ddNTP, a solid support, a spacer, or any combination thereof.

21. The method of any one of claims 1-19, wherein said single-stranded nucleic acid molecule with said cut and said guide polynucleotide bound thereto is used as a starting template for an amplification.

22. The method of claim 21, wherein said amplification is an isothermal amplification.

23. The method of any one of claims 1-22, wherein said enzyme exhibits a high-frequency endonuclease activity.

24. The method of claim 23, wherein said high-frequency endonuclease activity is from a large subunit of said enzyme.

25. The method of any one of claims 1-24, wherein said enzyme exhibits a low-frequency endonuclease activity.

26. The method of claim 25, wherein said low-frequency endonuclease activity is from a small subunit of said enzyme.

27. The method of any one of claims 1-26, wherein said enzyme exhibits at least two differential enzymatic activity rates.

28. The method of claim 27, wherein said at least two differential enzymatic activity rates comprise two differential endonuclease activity rates when cutting two different cutting sites.

29. The method of claim 27, wherein one of said two differential endonuclease activity rates comprises cutting said target sequence of said single-stranded nucleic acid molecule with low frequency.

30. The method of claim 27, wherein one of said two differential endonuclease activity rates comprises cutting said target binding region of said guide polynucleotide with high frequency.

31. The method of claim 27, wherein said two differential endonuclease activity rates are asymmetric or non-equal.

32. The method of claim 27, wherein said enzyme comprises BsmAI, Nt.BsmAI, Transcription Activator-Like Effector Nucleases, N.Bst9 I, N.BspD6I, Nt.BspQI, Nb.BbvCI, Nb.BsmI, Nb.BssSI, Nb.BsrDI, Nb.BtsI, Nt.AlwI, Nt.BbvCI, Nt.BstNBI, Nt.CviPII, Nb.Mva1269I, Nb.Bpu10I, and Nt.Bpu10I, a functional fragment thereof, or a combination thereof.

33. The method of any one of claims 27-32, wherein a temperature is changed over a course of said method.

34. The method of claim 33, wherein a first activity rate of said at least two differential enzymatic activity rates is favored at a first temperature, and a second activity rate of said at least two differential enzymatic activity rates is favored at a second temperature different from said first temperature.

35. The method of any one of claims 1-34, wherein said enzyme comprises two different active sites or endonuclease domains conferring at least two differential enzymatic activities.

36. The method of claim 35, wherein said target sequence comprises a recognition site specifically recognized by said enzyme or a first activity of said at least two differential enzymatic activities of said enzyme to introduce a cut.

37. The method of claim 35 or 36, wherein said target binding region of said guide polynucleotide comprises a recognition site specifically recognized by said enzyme or a second activity of said at least two differential enzymatic activities of said enzyme to introduce a cut.

38. The method of any one of claims 1-37, wherein said target binding region is at least about 12 to about 25 nucleotides in length.

39. The method of any one of claims 1-38, wherein a concentration of said guide polynucleotide is at least about 0.1 μM, at least about 1 μM, or about 0.1 μM to about 4 μM.

40. The method of any one of claims 1-39, wherein said non-target binding region comprises a palindromic sequence.

41. The method of any one of claims 1-40, wherein said non-target binding region is self-complementary.

42. The method of any one of claims 1-41, wherein said non-target binding region is at least about 12 nucleotides in length.

43. The method of any one of claims 1-42, wherein said single-stranded nucleic acid molecule is a single-stranded deoxyribonucleic acid (ssDNA) or a single-stranded ribonucleic acid (ssRNA).

44. The method of any one of claims 1-43, wherein said target binding region comprises at least one peptide nucleic acid (PNA) residue.

45. The method of any one of claims 3-44, wherein said polymerase has strand displacement activity.

46. The method of any one of claims 1-45, wherein said guide polynucleotide or said first guide polynucleotide further comprises an additional non-target binding region.

47. The method of claim 46, wherein said additional non-target binding region is located at a 5′ end of said guide polynucleotide or said first guide polynucleotide.

48. The method of claim 46 or 47, wherein said additional non-target binding region comprises an additional restriction endonuclease recognition sequence for an additional enzyme.

49. The method of claim 48, wherein said additional enzyme is the same or different from said enzyme.

50. The method of any one of claims 46-49, wherein said additional non-target binding region blocks said 3′ end of said guide polynucleotide or said first guide polynucleotide from extension.

51. The method of any one of claims 1-50, wherein said single-stranded nucleic acid molecule comprises two or more single-stranded nucleic acid molecules, each single-stranded nucleic acid molecule comprising a different target sequence.

52. The method of claim 51, wherein said two or more single-stranded nucleic acid molecules are contained within a single reaction mixture.

53. The method of any one of claims 4-52, wherein said method of amplifying a single-stranded nucleic acid molecule shortens a cycle threshold value or a time to result value in a nucleic acid amplification compared to a cycle threshold value or a time to result value in a nucleic acid amplification of an otherwise identical method of amplifying said single-stranded nucleic acid molecule without said guide complex.

54. The method of any one of claims 4-52, wherein said method of amplifying a single-stranded nucleic acid molecule shortens a cycle threshold value or a time to result value in a nucleic acid amplification compared to a cycle threshold value or a time to result value in an existing nucleic acid amplification method.

55. The method of claim 54, wherein said existing nucleic acid amplification method is selected from the group consisting of loop-mediated isothermal amplification (LAMP), helicase-dependent amplification (HDA), rolling circle amplification (RCA), multiple displacement amplification (MDA), recombinase polymerase amplification (RPA), and nucleic acid sequence-based amplification (NASBA).

56. The method of any one of claims 53-55, wherein said cycle threshold value is at most 30.

57. A polynucleotide-polypeptide complex comprising:
a single-stranded nucleic acid molecule having bound thereto a guide complex, wherein said guide complex comprises:
(i) a first guide polynucleotide comprising, from 5′ to 3′, a non-target binding region and a target binding region that hybridizes with a target sequence of said single-stranded nucleic acid molecule, and
(ii) a second guide polynucleotide that hybridizes with said non-target binding region of said first guide molecule to form a double-stranded binding region, wherein said double-stranded binding region comprises a restriction endonuclease recognition sequence for an enzyme that is a type IIs restriction enzyme.

58. A system of processing a single-stranded nucleic acid molecule comprising a target sequence, said system comprising:
said single-stranded nucleic acid molecule having bound thereto a guide complex comprising a guide polynucleotide, wherein said guide polynucleotide comprises:
(i) a non-target binding region comprising a restriction endonuclease recognition sequence for an enzyme that is a type IIs restriction enzyme,
(ii) a target binding region configured to hybridize to said target sequence, and
(iii) a blocked 3′ end non-extendable by a polymerase; and
said enzyme bound to said restriction endonuclease recognition sequence of said non-target binding region.

59. A kit comprising a guide complex or a guide polynucleotide of any one of claims 1-58.

60. The kit of claim 59, wherein said kit further comprises a probe or a dye for detecting an amplification product generated using said kit.

61. The kit of claim 59 or 60, wherein said kit further comprises an informational material describing an instruction of using said kit.

62. A system for processing a plurality of single-stranded nucleic acid molecules, each comprising a different target sequence, said system comprising:
a first single-stranded nucleic acid molecule wherein said first single-stranded nucleic acid molecule is bound to a first guide complex comprising a first guide polynucleotide, wherein said first guide polynucleotide comprises:
(i) a first non-target binding region comprising a first restriction endonuclease recognition sequence for an enzyme that is a type IIs restriction enzyme;
(ii) a first target binding region configured to hybridize to a first target sequence; and
(iii) a first blocked 3′ end non-extendable by a polymerase; and
a second single-stranded nucleic acid molecule wherein said second single-stranded nucleic acid molecule is bound to a second guide complex comprising a second guide polynucleotide, wherein said second guide polynucleotide comprises:
(i) a second non-target binding region comprising a second restriction endonuclease recognition sequence for said enzyme that is a type IIs restriction enzyme;
(ii) a second target binding region configured to hybridize to a second target sequence; and
(iii) a second blocked 3′ end non-extendable by a polymerase;
wherein said enzyme that is a type IIs restriction enzyme binds to said first restriction endonuclease recognition sequence of said first non-target binding region or said second restriction endonuclease recognition sequence of said second non-target binding region.

63. The system of claim 62, further comprising a third single-stranded nucleic acid molecule wherein said third single-stranded nucleic acid molecule is bound to a third guide complex comprising a third guide polynucleotide, wherein said third guide polynucleotide comprises:
(i) a third non-target binding region comprising a third restriction endonuclease recognition sequence for an enzyme that is a type IIs restriction enzyme;
(ii) a third target binding region configured to hybridize to a third target sequence; and
(iii) a third blocked 3′ end non-extendable by a polymerase;
wherein said enzyme that is a type IIs restriction enzyme binds to said third restriction endonuclease recognition sequence of said third non-target binding region.

64. The system of claim 62 or 63, further comprising a fourth single-stranded nucleic acid molecule wherein said fourth single-stranded nucleic acid molecule is bound to a fourth guide complex comprising a fourth guide polynucleotide, wherein said fourth guide polynucleotide comprises:
(i) a fourth non-target binding region comprising a fourth restriction endonuclease recognition sequence for an enzyme that is a type IIs restriction enzyme;
(ii) a fourth target binding region configured to hybridize to a fourth target sequence; and
(iii) a fourth blocked 3′ end non-extendable by a polymerase;
wherein said enzyme that is a type IIs restriction enzyme binds to said fourth restriction endonuclease recognition sequence of said fourth non-target binding region.

65. The system of any one of claims 62-64, wherein said first single-stranded nucleic acid molecule and said second single-stranded nucleic acid molecule are from different samples.

66. The system of claim 65, wherein said different samples comprise samples obtained from a bacterium, a virus, a human, or any combination thereof.

67. The system of claim 66, wherein said bacterium is selected from the group consisting of Neisseria gonorrhoeae, Chlamydia trachomatis, and Trichomonas vaginalis.

68. The system of claim 66, wherein said virus is selected from the group consisting of a double-stranded DNA virus, a single-stranded DNA virus, a double-stranded RNA virus, a single-stranded RNA virus, a positive sense single-stranded reverse transcriptase virus, and a double-stranded DNA reverse transcriptase virus.


SEQUENCE LISTING (45 sequences)

SEQ ID NO: 1 - DNA, 34 bp, synthetic construct
  source 1..34: mol_type = other DNA
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 3'-phosphate 2'-O-methyl modified nucleotide
  misc_feature 1..28: note = DNA
  misc_feature 29..34: note = RNA
gactcggccgagtcggtgcaatttcaagtgtttt

SEQ ID NO: 2 - DNA, 31 bp, synthetic construct
  source 1..31: mol_type = other DNA
  modified_base 26: mod_base = OTHER; note = 2'-O-methyl modified nucleotide
  modified_base 27: mod_base = OTHER; note = 2'-O-methyl modified nucleotide
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 3'-phosphate 2'-O-methyl modified nucleotide
  misc_feature 1..25: note = DNA
  misc_feature 26..31: note = RNA
gactcggccgagtccacttcagcctcaactt

SEQ ID NO: 3 - DNA, 35 bp, synthetic construct
  source 1..35: mol_type = other DNA
  modified_base 1: mod_base = OTHER; note = 5' 6-Carboxyfluorescein modified nucleotide
  modified_base 35: mod_base = OTHER; note = 3' black hole quencher 1 modified nucleotide
cgcgcaacaagtgtttcaaatgatatccttgcgcg

SEQ ID NO: 4 - DNA, 30 bp, synthetic construct
  source 1..30: mol_type = other DNA
  modified_base 1: mod_base = OTHER; note = 5' Cyanine-5 modified nucleotide
  modified_base 30: mod_base = OTHER; note = 3' black hole quencher 2 modified nucleotide
ccgcaagtgttttaaatgatatccttgcgg

SEQ ID NO: 5 - DNA, 302 bp, synthetic construct
  source 1..302: mol_type = other DNA
gttctctatgagaaccaaaaattgattgccaaccaatttaatagtgctattggcaaaattcaagagtcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagc

SEQ ID NO: 6 - DNA, 34 bp, synthetic construct; identical to SEQ ID NO: 1
  source 1..34: mol_type = other DNA
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 3'-phosphate 2'-O-methyl modified nucleotide
  misc_feature 1..28: note = DNA
  misc_feature 29..34: note = RNA

SEQ ID NO: 7 - DNA, 31 bp, synthetic construct; identical to SEQ ID NO: 2
  source 1..31: mol_type = other DNA
  modified_base 26: mod_base = OTHER; note = 2'-O-methyl modified nucleotide
  modified_base 27: mod_base = OTHER; note = 2'-O-methyl modified nucleotide
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 3'-phosphate 2'-O-methyl modified nucleotide
  misc_feature 1..25: note = DNA
  misc_feature 26..31: note = RNA

SEQ ID NO: 8 - DNA, 35 bp, synthetic construct; identical to SEQ ID NO: 3
  source 1..35: mol_type = other DNA
  modified_base 1: mod_base = OTHER; note = 5' 6-Carboxyfluorescein modified nucleotide
  modified_base 35: mod_base = OTHER; note = 3' black hole quencher 1 modified nucleotide

SEQ ID NO: 9 - DNA, 30 bp, synthetic construct; identical to SEQ ID NO: 4
  source 1..30: mol_type = other DNA
  modified_base 1: mod_base = OTHER; note = 5' Cyanine-5 modified nucleotide
  modified_base 30: mod_base = OTHER; note = 3' black hole quencher 2 modified nucleotide

SEQ ID NO: 10 - DNA, 302 bp, synthetic construct
  source 1..302: mol_type = other DNA
gttctctatgagaaccaaaaattgattgccaaccaatttaatagtgctattggcaaaattcaagagtcactttcttccacagcaagtgcacttggaaaacttcaagatgtggtcaaccaaaatgcacaagctttaaacacgcttgttaaacaacttagctccaattttggtgcaatttcaagtgtttcaaatgatatcctttcacgtcttgacaaagttgaggctgaagtgcaaattgataggttgatcacaggcagacttcaaagtttgcagacatatgtgactcaacaattaattagagc

SEQ ID NO: 11 - DNA, 34 bp, synthetic construct; identical to SEQ ID NO: 1
  source 1..34: mol_type = other DNA
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 3'-phosphate 2'-O-methyl modified nucleotide
  misc_feature 1..28: note = DNA
  misc_feature 29..34: note = RNA

SEQ ID NO: 12 - DNA, 31 bp, synthetic construct; identical to SEQ ID NO: 2
  source 1..31: mol_type = other DNA
  modified_base 26: mod_base = OTHER; note = 2'-O-methyl modified nucleotide
  modified_base 27: mod_base = OTHER; note = 2'-O-methyl modified nucleotide
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 3'-phosphate 2'-O-methyl modified nucleotide
  misc_feature 1..25: note = DNA
  misc_feature 26..31: note = RNA

SEQ ID NO: 13 - DNA, 70 bp, synthetic construct
  source 1..70: mol_type = other DNA
  modified_base 1: mod_base = OTHER; note = 5' 6-Carboxyfluorescein modified nucleotide
  modified_base 70: mod_base = OTHER; note = 3' black hole quencher 1 modified nucleotide
cgcgcaacaagtgtcttaaatgatatccttgcgcgcgcgcaacaagtgtcttaaatgatatccttgcgcg

SEQ ID NO: 14 - DNA, 30 bp, synthetic construct; identical to SEQ ID NO: 4
  source 1..30: mol_type = other DNA
  modified_base 1: mod_base = OTHER; note = 5' Cyanine-5 modified nucleotide
  modified_base 30: mod_base = OTHER; note = 3' black hole quencher 2 modified nucleotide

SEQ ID NO: 15 - DNA, 302 bp, synthetic construct; identical to SEQ ID NO: 5
  source 1..302: mol_type = other DNA

SEQ ID NO: 16 - DNA, 34 bp, synthetic construct; identical to SEQ ID NO: 1
  source 1..34: mol_type = other DNA
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 3'-phosphate 2'-O-methyl modified nucleotide
  misc_feature 1..28: note = DNA
  misc_feature 29..34: note = RNA

SEQ ID NO: 17 - DNA, 31 bp, synthetic construct; identical to SEQ ID NO: 2
  source 1..31: mol_type = other DNA
  modified_base 26: mod_base = OTHER; note = 2'-O-methyl modified nucleotide
  modified_base 27: mod_base = OTHER; note = 2'-O-methyl modified nucleotide
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 3'-phosphate 2'-O-methyl modified nucleotide
  misc_feature 1..25: note = DNA
  misc_feature 26..31: note = RNA

SEQ ID NO: 18 - DNA, 70 bp, synthetic construct; identical to SEQ ID NO: 13
  source 1..70: mol_type = other DNA
  modified_base 1: mod_base = OTHER; note = 5' 6-Carboxyfluorescein modified nucleotide
  modified_base 70: mod_base = OTHER; note = 3' black hole quencher 1 modified nucleotide

SEQ ID NO: 19 - DNA, 30 bp, synthetic construct; identical to SEQ ID NO: 4
  source 1..30: mol_type = other DNA
  modified_base 1: mod_base = OTHER; note = 5' Cyanine-5 modified nucleotide
  modified_base 30: mod_base = OTHER; note = 3' black hole quencher 2 modified nucleotide

SEQ ID NO: 20 - DNA, 302 bp, synthetic construct
  source 1..302: mol_type = other DNA
gttctctatgagaaccaaaaattgattgccaaccaatttaatagtgctattggcaaaattcaagagtcactttcttccacagcaagtgcacttggaaaacttcaagatgtggtcaaccaaaatgcacaagctttaaacacgcttgttaaacaacttagctccaattttggtgcaatttcaagtgtcttaaatgatatcctttcacgtcttgacaaagttgaggctgaagtgcaaattgataggttgatcacaggcagacttcaaagtttgcagacatatgtgactcaacaattaattagagc

SEQ ID NO: 21 - DNA, 32 bp, synthetic construct
  source 1..32: mol_type = other DNA
gactcgatatcgagtcagctgagactgcgcct

SEQ ID NO: 22 - DNA, 32 bp, synthetic construct; identical to SEQ ID NO: 21
  source 1..32: mol_type = other DNA
  modified_base 32: mod_base = OTHER; note = 3'-phosphate modified nucleotide

SEQ ID NO: 23 - DNA, 32 bp, synthetic construct; identical to SEQ ID NO: 21
  source 1..32: mol_type = other DNA
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  misc_feature 1..27: note = DNA
  misc_feature 28..32: note = RNA

SEQ ID NO: 24 - DNA, 32 bp, synthetic construct; identical to SEQ ID NO: 21
  source 1..32: mol_type = other DNA
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 3'-phosphate 2'-O-methyl modified nucleotide
  misc_feature 1..27: note = DNA
  misc_feature 28..32: note = RNA

SEQ ID NO: 25 - DNA, 32 bp, synthetic construct; identical to SEQ ID NO: 21
  source 1..32: mol_type = other DNA
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  misc_feature 1..27: note = DNA
  misc_feature 28..31: note = RNA
  misc_feature 32: note = DNA

SEQ ID NO: 26 - DNA, 32 bp, synthetic construct
  source 1..32: mol_type = other DNA
  modified_base 32: mod_base = OTHER; note = 3'-phosphate modified nucleotide
gagagcattacgagtcagctgagactgcgcct

SEQ ID NO: 27 - DNA, 32 bp, synthetic construct; identical to SEQ ID NO: 21
  source 1..32: mol_type = other DNA
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  misc_feature 1..27: note = DNA
  misc_feature 28..32: note = RNA

SEQ ID NO: 28 - DNA, 32 bp, synthetic construct; identical to SEQ ID NO: 26
  source 1..32: mol_type = other DNA

SEQ ID NO: 29 - DNA, 32 bp, synthetic construct; identical to SEQ ID NO: 21
  source 1..32: mol_type = other DNA
  modified_base 32: mod_base = OTHER; note = 3'-phosphate modified nucleotide

SEQ ID NO: 30 - DNA, 32 bp, synthetic construct; identical to SEQ ID NO: 26
  source 1..32: mol_type = other DNA
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  misc_feature 1..27: note = DNA
  misc_feature 28..32: note = RNA

SEQ ID NO: 31 - DNA, 32 bp, synthetic construct; identical to SEQ ID NO: 21
  source 1..32: mol_type = other DNA

SEQ ID NO: 32 - DNA, 45 bp, synthetic construct
  source 1..45: mol_type = other DNA
gggagtggagtgaagaccttcccaagggacattcactccactccc

SEQ ID NO: 33 - DNA, 46 bp, synthetic construct
  source 1..46: mol_type = other DNA
tcactccactccccacgagctgagtgcgtcctggggagtggagtga

SEQ ID NO: 34 - DNA, 34 bp, synthetic construct
  source 1..34: mol_type = other DNA
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 3'-phosphate 2'-O-methyl modified nucleotide
  misc_feature 1..27: note = DNA
  misc_feature 28..34: note = RNA
gactcggccgagtccacgagctgagtgcgtcctg

SEQ ID NO: 35 - DNA, 33 bp, synthetic construct
  source 1..33: mol_type = other DNA
  modified_base 27: mod_base = OTHER; note = 2'-O-methyl modified nucleotide
  modified_base 28: mod_base = OTHER; note = 2'-O-methyl modified nucleotide
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 3'-phosphate 2'-O-methyl modified nucleotide
  misc_feature 1..26: note = DNA
  misc_feature 27..33: note = RNA
gactcggccgagtcagaccttcccaagggacat

SEQ ID NO: 36 - DNA, 36 bp, synthetic construct
  source 1..36: mol_type = other DNA
gaagagcgatatcgctcttcagctgagactgcgcct

SEQ ID NO: 37 - DNA, 36 bp, synthetic construct; identical to SEQ ID NO: 36
  source 1..36: mol_type = other DNA
  modified_base 36: mod_base = OTHER; note = 3'-phosphate modified nucleotide

SEQ ID NO: 38 - DNA, 36 bp, synthetic construct; identical to SEQ ID NO: 36
  source 1..36: mol_type = other DNA
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 3'-phosphate 2'-O-methyl modified nucleotide
  misc_feature 1..30: note = DNA
  misc_feature 31..36: note = RNA

SEQ ID NO: 39 - DNA, 36 bp, synthetic construct; identical to SEQ ID NO: 36
  source 1..36: mol_type = other DNA
  modified_base 24: mod_base = OTHER; note = 2'-O-methyl modified nucleotide
  modified_base 25: mod_base = OTHER; note = 2'-O-methyl modified nucleotide
  misc_feature 1..23: note = DNA
  misc_feature 24..25: note = RNA
  misc_feature 26..36: note = DNA

SEQ ID NO: 40 - DNA, 36 bp, synthetic construct; identical to SEQ ID NO: 36
  source 1..36: mol_type = other DNA
  modified_base 24: mod_base = OTHER; note = 2'-O-methyl modified nucleotide
  modified_base 25: mod_base = OTHER; note = 2'-O-methyl modified nucleotide
  modified_base 36: mod_base = OTHER; note = 3'-phosphate modified nucleotide
  misc_feature 1..23: note = DNA
  misc_feature 24..25: note = RNA
  misc_feature 26..36: note = DNA

SEQ ID NO: 41 - DNA, 36 bp, synthetic construct; identical to SEQ ID NO: 36
  source 1..36: mol_type = other DNA
  modified_base 24: mod_base = OTHER; note = 2'-O-methyl modified nucleotide
  modified_base 25: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 3'-phosphate 2'-O-methyl modified nucleotide
  misc_feature 1..23: note = DNA
  misc_feature 24..25: note = RNA
  misc_feature 26..30: note = DNA
  misc_feature 31..36: note = RNA

SEQ ID NO: 42 - DNA, 42 bp, synthetic construct
  source 1..42: mol_type = other DNA
  modified_base 1: mod_base = OTHER; note = 5' Iowa Black FQ modified nucleotide
  modified_base 20: mod_base = OTHER; note = Fluorescein modified nucleotide
accggcggttaaccgccggtaggcgcagtctcagctcgtgct

SEQ ID NO: 43 - DNA, 36 bp, synthetic construct; identical to SEQ ID NO: 36
  source 1..36: mol_type = other DNA
  modified_base 24: mod_base = OTHER; note = 2'-O-methyl modified thymine
  modified_base 25: mod_base = OTHER; note = 2'-O-methyl modified nucleotide
  modified_base 36: mod_base = OTHER; note = 3'-phosphate modified nucleotide
  misc_feature 1..23: note = DNA
  misc_feature 24..25: note = RNA
  misc_feature 26..36: note = DNA

SEQ ID NO: 44 - DNA, 36 bp, synthetic construct; identical to SEQ ID NO: 36
  source 1..36: mol_type = other DNA
  modified_base 24: mod_base = OTHER; note = 2'-O-methyl modified thymine
  modified_base 25: mod_base = OTHER; note = 2'-O-methyl modified nucleotide
  misc_feature 1..23: note = DNA
  misc_feature 24..25: note = RNA
  misc_feature 26..36: note = DNA

SEQ ID NO: 45 - DNA, 42 bp, synthetic construct; identical to SEQ ID NO: 42
  source 1..42: mol_type = other DNA
  modified_base 1: mod_base = OTHER; note = 5' Iowa Black FQ modified nucleotide
  modified_base 20: mod_base = OTHER; note = Iowa Black FQ quenched fluorescein modified nucleotide